SEQUENCE LISTING (141 sequences)

SEQ ID NO: 1 - DNA, 820 bp, Borrelia burgdorferi
caaaagatatacgtagaaatagaaagacgtattgaaaaccacaattttttgttttacaaagatgaatctttagtacaactacaagacgcactgtctagcgcaacaacttctttaagtgcacttactcagagcaataatgataggggaagtggccttttatcttcttttttgagaaaacaaaattcaaacaatcatagtaaagatatttctaatttaagaacccttaatgactcattatcacaggagcttgctaggctaaaaagcaatctaaataatgagggaatgttttatacggccacccctagtgctagtttagaggttattaaatacgaccttagttacttaaaaaggaggctttagcattaattaaggcaaaaattggtgcagatactaaagaacccttaaccagaagtattaatgagcaggctaaagggctaggaaatgatggtaaaggggataggagtaattattacgattttctcaaaggtgtacaagaacaagttgagaactcttgtaatttaaaacttacaaagtattttgggcttgatatgaaatttaattcgctgattatgttaagtgaagaacaaaaagtggaaagagatataaagctaattgagctttacagtaaatataaccagcttatacaaagtagctcctttgataatgaggagttagcgattttaaaagagaaattattctcattttgagaaaaggagttaaaagatgcctgagaaagaagaaaaagaagacctgcaagcacaagataaagaagagcagcaaaataaggctgatactaaagttataagtgcgcaggaatttg

SEQ ID NO: 2 - protein, 119 aa, Borrelia burgdorferi
QKIYVEIERRIENHNFLFYKDESLVQLQDALSSATTSLSALTQSNNDRGSGLLSSFLRKQNSNNHSKDISNLRTLNDSLSQELARLKSNLNNEGMFYTATPSASLEVIKYDLSYLKRRL

SEQ ID NO: 3 - DNA, 1224 bp, Borrelia burgdorferi
gtgtgtgatttaagaaaaacaaaactaatagataaaataagttcactagaactatacaaatactcaatattttttagaaattacattgaaaatgtagcagaagattgtctcaagaacggacttattcttgagagtgctgcccacaatgttagtgaggttgaacttgctaggttaaaggtacagcttaagaatgctctgcttaattgtattataagctaccgttttcatgggattggctatgttttagtaaaaaccaaagataccctaatagatctcgaacaacccgttaatatagaattacctattggttttgaataccttgattatgaatatgtaagagatttgggagttgattttgatcatataacctataaagtaaaatccaacaataagaacaattctttagacgcagttaaaatacataaaagtcgacttatcatatatgaaaactttgattatatcttaaaaagatatgttccgtgttataccgaaagctttttactagatatttatttatttgaaaagatatacgtagaaatagaaagacgtattgaaaaccacaatttcttgttttataaagatgaatctttagtacaactacaagacgcactctctagtgcaacaacttctttaagtgcacttactcagagcaataatgataggggaagtggcattttatcttcttttttgagaaaacaaaattcaaacaatcatagtaaagatatttctaatttaagaaaccttaatgactcattatcacaggagcttgctaggctaaaaagcaatctaaataatgagggaatgttttatacggccacccctagtgctagtttagaggttattaaatacgatcttagctacttaaaggaggctttagcattaattaaggcaaaaattggtgcagatactaaagagcccttaaccagaagttttaatgagcaggctaaaggactaggaaatgatggtaaaggtgataggagtaattattacgattttctcaaaggtgtacaagaacaagttgagaactcttgtaatttaaaacttacaaagtattttgggcttgatatgaagtttaattctctgattatgttaagtgaagaacaaaaagtggaaagagatataaagctaattgagctttacagtaaatataaccagcttatacaaagtagctcatttaataatgaggagctagcgatgttaaaagagaaattattctcattttga

SEQ ID NO: 4 - protein, 407 aa, Borrelia burgdorferi
MCDLRKTKLIDKISSLELYKYSIFFRNYIENVAEDCLKNGLILESAAHNVSEVELARLKVQLKNALLNCIISYRFHGIGYVLVKTKDTLIDLEQPVNIELPIGFEYLDYEYVRDLGVDFDHITYKVKSNNKNNSLDAVKIHKSRLIIYENFDYILKRYVPCYTESFLLDIYLFEKIYVEIERRIENHNFLFYKDESLVQLQDALSSATTSLSALTQSNNDRGSGILSSFLRKQNSNNHSKDISNLRNLNDSLSQELARLKSNLNNEGMFYTATPSASLEVIKYDLSYLKEALALIKAKIGADTKEPLTRSFNEQAKGLGNDGKGDRSNYYDFLKGVQEQVENACNLKLTKYFGLDMKFNSLIMLSEEQKVERDIKLIELYSKYNQLIQSSSFDNEELAILKEKLFSF

SEQ ID NO: 5 - DNA, 275 bp, Borrelia burgdorferi
cttcaaggttttgtggtgccaagctatgttccaatttccagcattagtgagcttaagggtaaaggtgataagtttaaaaacaaaatgattggcatagatgctggtgcgggaactcaaattgttacagaacaagcgcttaattattatggattaagtaaagagtatgagctagttccttcaagtgagagtgttatgcttgcaagtttagattcttcaataaagagaaacgaatggattttagttcctttgtggaagcctcattgggctttttctag

SEQ ID NO: 6 - protein, 91 aa, Borrelia burgdorferi
LQGFVVPSYVPISSISELKGKGDKFKNKMIGIDAGAGTQIVTEQALNYYGLSKEYELVPSSESVMLASLDSSIKRNEWILVPLWKPHWAFS

SEQ ID NO: 7 - DNA, 873 bp, Borrelia burgdorferi
atgtataaattatttttattttttattatttttatgtttttgtcttgtgatgaaaaaaagagttcaaagaatttaaaatcggtaaaaattggatatgtgaattggggtggagaaacggcagctacaaatgtattaaaggttgtttttgagaaaatgggctacaatgcagaaatattttcagttactacgtctataatgtatcaatacttagcatctggaaagatagacggtacggtgtcttcttgggttcctacagccgataaattttattatgaaaaactgaaaacaaagtttgttgatcttggtgcaaattatgaaggaaccattcaaggttttgtggtgccaagctatgttccaatttccagcattagtgagcttaagggtaaaggtgataagtttaaaaacaaaatgattggcatagatgctggtgcgggaactcaaattgttacagaacaagcgcttaattattatggattaagtaaagagtatgagctagttccttcaagtgagagtgttatgcttgcaagtttagattcttcaataaagagaaacgaatggattttagttcctttgtggaagcctcattgggctttttctaggtatgatattaagtttcttgatgatcctgatttaattatggggggaattgagagcgtgcatactcttgttagacttggtcttgaaaatgatgattttgatgcatattatgtttttgatcatttttattggagcgatgatttaatattgcccttaatggataaaaatgataaagagccaggcaaagaataccgcaatgcggttgaatttgttgaaaagaataaagagattgtaaagacgtgggttccagaaaaatataagaccttatttgattaa

SEQ ID NO: 8 - protein, 290 aa, Borrelia burgdorferi
MYKLFLFFIIFMFLSCDEKKSSKNLKSVKIGYVNWGGETAATNVLKVVFEKMGYNAEIFSVTTSIMYQYLASGKIDGTVSSWVPTADKFYYEKLKTKFVDLGANYEGTIQGFVVPSYVPISSISELKGKGDKFKNKMIGIDAGAGTQIVTEQALNYYGLSKEYELVPSSESVMLASLDSSIKRNEWILVPLWKPHWAFSRYDIKFLDDPDLIMGGIESVHTLVRLGLENDDFDAYYVFDHFYWSDDLILPLMDKNDKEPGKEYRNAVEFVEKNKEIVKTWVPEKYKTLFD

SEQ ID NO: 9 - DNA, 612 bp, Borrelia burgdorferi
cgaaagtttccttttgatttgaatattgacatgactgtcaatatgcctttgcaaaaaaaatctcatctcaagcgagatttgaaagaattgctttcatatatgcctgagcatatttgttttagcgattttatatgtgaagaggaaggctttgtcttgagagattttgataacagtattgattcggaaaaactgtggttttgtgctctggagtgtctagaatccaatggctacattaattatgaaattactaattttgcattaaaggggcatgagagcaggcacaataagctaaattgggagttaaagccgcatttaggattaggattgtatgctgtaagtttgcttttttgtaatgacaagaataataatgtaagagctttgattagaaaaactggtagttttgtcaaagcaaataatcacttagtaacgtttgaattgttagaggatttggagttttttgtttatcattttattcaagggcttggaaccattcaaggtgtcagcttgagggctcttaggcttagatttgagtataatgaaaaacaattttttcagtttattaattactgctcaactttaagtaaaaagtttgtttttgatgataatattatg

SEQ ID NO: 10 - protein, 204 aa, Borrelia burgdorferi
RKFPFDLNIDMTVNMPLQKKSHLKRDLKELLSYMPEHICFSDFICEEEGFVLRDFDNSIDSEKLWFCALECLESNGYINYEITNFALKGHESRHNKLNWELKPHLGLGLYAVSLLFCNDKNNNVRALIRKTGSFVKANNHLVTFELLEDLEFFVYHFIQGLGTIQGVSLRALRLRFEYNEKQFFQFINYCSTLSKKFVFDDNIM

SEQ ID NO: 11 - DNA, 1134 bp, Borrelia burgdorferi
atgagagtagatcttttacctcttgtcgagttaagtctttatattaatttgtcattttgttgtaaagattttagcatttttaatagaattttagaggaattaaaatgtcatttaatcttgctgggtcatccaattataaaaacactttacattaagcacgtagatttttgtttatctaggcaagataatttaaaatttattttcacttctttgtccaagtatattaatttggagttattagaagaatttactttagaaattattccgggttatgttgattttgaaaaattcaaacttttggatgaattttgtattactagaattaatcttaatgttcaaagtttttctttagagtttagaaagattgtggggatacccgaaatttcttataaaaaattgaatattttgattaacaatattagaaagtttccttttgatttgaatattgacatgactgtcaatatgcctttgcaaaaaaaatctcatctcaagcgagatttgaaagaattgctttcatatatgcctgagcatatttgttttagcgattttatatgtgaagaggaaggctttgtcttgagagattttgataacagtattgattcggaaaaactgtggttttgtgctctggagtgtctagaatccaatggctacattaattatgaaattactaattttgcattaaaggggcatgagagcaggcacaataagctaaattgggagttaaagccgcatttaggattaggattgtatgctgtaagtttgcttttttgtaatgacaagaataataatgtaagagctttgattagaaaaactggtagttttgtcaaagcaaataatcacttagtaacgtttgaattgttagaggatttggagttttttgtttatcattttattcaagggcttggaaccattcaaggtgtcagcttgagggctcttaggcttagatttgagtataatgaaaaacaattttttcagtttattaattactgctcaactttaagtaaaaagtttgtttttgatgataatattatgctattaaaagggagagaaaggtttaagttaaatttttatttagtaaaaattataaaccattttaatgataacttttttaaagttaagcttaggcttccttga

SEQ ID NO: 12 - protein, 377 aa, Borrelia burgdorferi
MRVDLLPLVELSLYINLSFCCKDFSIFNRILEELKCHLILLGHPIIKTLYIKHVDFCLSRQDNLKFIFTSLSKYINLELLEEFTLEIIPGYVDFEKFKLLDEFCITRINLNVQSFSLEFRKIVGIPEISYKKLNILINNIRKFPFDLNIDMTVNMPLQKKSHLKRDLKELLSYMPEHICFSDFICEEEGFVLRDFDNSIDSEKLWFCALECLESNGYINYEITNFALKGHESRHNKLNWELKPHLGLGLYAVSLLFCNDKNNNVRALIRKTGSFVKANNHLVTFELLEDLEFFVYHFIQGLGTIQGVSLRALRLRFEYNEKQFFQFINYCSTLSKKFVFDDNIMLLKGRERFKLNFYLVKIINHFNDNFFKVKLRLP

SEQ ID NO: 13 - DNA, 523 bp, Borrelia burgdorferi
caaattactcagcatattttagctagcattcttgaagaatcaattatttttattttaagaatattttttattgcatatattgaagataacgacatttttaagaaaatattacaagaaaataagctatacagatcttctatatcttttagatattttttttatgatgaaaatacaaaaaagaaattagaatataaaaaaataataacaattttcaatttacttgataaaggaagtgatgcaataaagtttcctgtatttaatggagggttattttcagaagataaggttaaatatttaaataatgaaggtttgctcagtattagcgagattgaagaaatactagttaaaatgcttttctttgaagaaaaaaatattaaagatgaaaaatttgtaaagtattcaaggttagatcctaaaagttttggagaattatacgaaactctacttgaatatgacctaagaattgcagataccactgttcatcgtattattgaagacggagtttaccttattcgtactgaag

SEQ ID NO: 14 - protein, 174 aa, Borrelia burgdorferi
QITQHILASILEESIIFILRIFFIAYIEDNDIFKKILQENKLYRSSISFRYFFYDENTKKKLEYKKIITIFNLLDKGSDAIKFPVFNGGLFSEDKVKYLNNEGLLSISEIEEILVKMLFFEEKNIKDEKFVKYSRLDPKSFGELYETLLEYDLRIADTTVHRIIEDGVYLIRTE

SEQ ID NO: 15 - DNA, 3834 bp, Borrelia burgdorferi
atgaaaactaatgatatcgtaaaaacaaataatcctaatatatctctttataaacaattatcaaaagactttataaaaaaggaaaatattaataagctaaaagacttttttattcttataaaaaataaacttttttcaatagatgataattccacagaagcaaatatagagtctttgctaaagtatatatttgaagaactaaattattcagtagaacaacaaaaagccggtcaaatagaaggagtggagtctagagtagatatactgctttttgaaaacgacaaagacaaagcatcctttaataataaattaaaagaagctaaaaaaaataatgaacctattcctatcgaagatatcttgattatagcagaggttaagcgcccaacatttagttttgatgctaaagataaattaaaagaatcagaagatcagctatatagatatctaaatcaatatcaaaaacattatgggatactttcaaatggaaaggtatggagattatatgacaaatcgaaagtactttatggagaaaaaagatatattgaattcaattttkctaaaattgaagaaaaagaagaatataaggaacaagaatggtttgttttattcatctaccttataagaaaagaaagatatctaaagacaagtaatgtaatagaggttgaaaaagaacaaatagctaaagaaaaagagataattcaaaaaactctaaaagagatactttatgagagacccgacgactctatagtatttaaaattgcaaaaaatatatatgacaaagaatttaaattatcaggcaaagaaattactcagcatattttagctagcattcttgaagaatcaattatttttattttaagaatattttttattgcatatattgaagataacgacatttttaagaaaatattacaagaaaataagctatacagatcttctatatcttttagatattttttttatgatgaaaatacaaaaaagaaattagaatataaaaaaataataacaattttcaatttacttgataaaggaagtgatgcaataaagtttcctgtatttaatggagggttattttcagaagataaggttaaatatttaaataatgaaggtttgctcagtattagcgagattgaagaaatactagttaaaatgcttttctttgaagaaaaaaatattaaagatgaaaaatttgtaaagtattcaaggttagatcctaaaagttttggagaattatacgaaactctacttgaatatgacctaagaattgcagataccactgttcatcgtattattgaagacggagtttaccttattcgtactgaagaagagcttgaaaataagaaagtaaacaaaattgctacatatcttaaaggtaatatttatcttacatctagatcacttgatagaaagaaaagtggggcatattatactccagatgacttgactgattttatggtcatatcatcaattgaagagcagcttaaaaccaaatcccctttagatataaagataattgataattcttgtggatcagggcattttttaatttcttgtctagattacttaacagaaaaggtatggtacgagctagataaatttgaagatgtaaaaaaagaacttgatgaagaatatagggttattattgaagaaagtgaagagtatgatgttcaagatagtataagtaaagaattagtacttaaaaggatgttactaaagragtgtatttatggtgttgatattaatcccatttcagttgaaattactatgctaagtttgtggattaatacttttatttttggaacaccactaagctttattgaacatcatataaaagcaggaaatgccctgctaggatataccaaagatgaattctttgatattgtaaaaaagaaatttgaaagtggattttctttgtttaaaaaaagaattaaagaaattataactattttagaagatatttatcaaaaaattaaaggtattaatgatactactaaagaagatatagaaaaatctaaaaagatatacaaagaatatgaagaaagtaaagatatagataatttaagaataatattttctttaattaaactttattcattatcttttgataaatctttaaatatggaatttagtgatattgcgtctgtaattagtttaattgaaaatattttgggcaataaaacttctagtgaagataaagaaaaaatagaaaaaattagaaaattaagtagctactataaattttttcactatggaattgaatttccagatattcaagaaggatttgatattgtaattggaaatcctccatgggagaaaactaaatttaatgaaacagagtttttttcaaaacatattcctaactacagaaaactaggcataaaagaacaaaatatcataaaacaagaaatacttagtaaagataatcatcctttgagtattgaatacaatgaagaaaaaaatagtataattgccattaataatatttataaatttgattttaaatgctttactagtggtggagacccaaatctttttagatactttgttacatttaatttaaaattaataaaagaaaaaggtaatttaacttatttagttccttcagctatttggaatgaatctagttctagaatactaagaaaacatatatttgctcgctataaacttaactatatttatcaatttgaaaacaaaaaaagatttaaagatgtgcattcaagttttaaatttgcaatatttcaacttagtaatattaaagaatctacatcgagctttaaagcaaaattcatgattcagagtagtgataatattttaaaagaaataactagggatttgaaagatagtaaagacgatgcttataaaggaattgaattaaatataaatcaaattaagaagctatctcctattcaagaatcaataatagaattcaaagacaatgaagaatttactcttattaataaaatgtttagcaaatttagtgctcttggtgaaggatatattgattttaaaaaaggcttagatccaagcattaagaatcgtaaatctttattaaaagaatgtaataataaaaatcttatatttctttattctggagctaatattcatcaatttaattcaagattttttgaagataaagatgcaaaagaaagctctaaattgctatggatagataaagaggatttggaaaaggtattaactaaagataaccaatatcaaaccgaaagagtattttatagagcaattgcaagcaacacaaatgaaagaactatgattagtactttatctcctggaaattgttattgtgtgaattcaatatatataaatgatgagaaaacaccaatatcactttataaaaaattatttattatatctattttcaactcatttgtatttgactttttgttaagaagatttgttgattcaaatgtgctaaaatcatgcctttatcaatgcccaatgcctcaacccgaagaaaaagaaattttaagtaatcctttatatttaaatcttgcaaaaaatacttctttactgatagccaaaaatgatcccgaaaactttaaatatttactttacttagaatattttaagtttgataaagaaaaagttaataaaatactgaaactagataaagaagatgaattttttaaagaaaaagaaaatgaaaataattttattatagctagtctttactcattagctaaggaagattttatcaccttgcttggtgattttaaggcattaaagaataaaaaaaaaggagaagattatatttcttctttaataaaaggatatgataattatttactaaataataaaattttttatcataaataa

SEQ ID NO: 16 - protein, 1277 aa, Borrelia burgdorferi
  MOD_RES (186): Xaa = anything
MKTNDIVKTNNPNISLYKQLSKDFIKKENINKLKDFFILIKNKLFSIDDNSTEANIESLLKYIFEELNYSVEQQKAGQIEGVESRVDILLFENDKDKASFNNKLKEAKKNNEPIPIEDILIIAEVKRPTFSFDAKDKLKESEDQLYRYLNQYQKHYGILSNGKVWRLYDKSKVLYGEKRYIEFNFXKIEEKEEYKEQEWFVLFIYLIRKERYLKTSNVIEVEKEQIAKEKEIIQKTLKEILYERPDDSIVFKIAKNIYDKEFKLSGKEITQHILASILEESIIFILRIFFIAYIEDNDIFKKILQENKLYRSSISFRYFFYDENTKKKLEYKKIITIFNLLDKGSDAIKFPVFNGGLFSEDKVKYLNNEGLLSISEIEEILVKMLFFEEKNIKDEKFVKYSRLDPKSFGELYETLLEYDLRIADTTVHRIIEDGVYLIRTEEELENKKVNKIATYLKGNIYLTSRSLDRKKSGAYYTPDDLTDFMVISSIEEQLKTKSPLDIKIIDNSCGSGHFLISCLDYLTEKVWYELDKFEDVKKELDEEYRVIIEESEEYDVQDSISKELVLKRMLLKXCIYGVDINPISVEITMLSLWINTFIFGTPLSFIEHHIKAGNALLGYTKDEFFDIVKKKFESGFSLFKKRIKEIITILEDIYQKIKGINDTTKEDIEKSKKIYKEYEESKDIDNLRIIFSLIKLYSLSFDKSLNMEFSDIASVISLIENILGNKTSSEDKEKIEKIRKLSSYYKFFHYGIEFPDIQEGFDIVIGNPPWEKTKFNETEFFSKHIPNYRKLGIKEQNIIKQEILSKDNHPLSIEYNEEKNSIIAINNIYKFDFKCFTSGGDPNLFRYFVTFNLKLIKEKGNLTYLVPSAIWNESSSRILRKHIFARYKLNYIYQFENKKRFKDVHSSFKFAIFQLSNIKESTSSFKAKFMIQSSDNILKEITRDLKDSKDDAYKGIELNINQIKKLSPIQESIIEFKDNEEFTLINKMFSKFSALGEGYIDFKKGLDPSIKNRKSLLKECNNKNLIFLYSGANIHQFNSRFFEDKDAKESSKLLWIDKEDLEKVLTKDNQYQTERVFYRAIASNTNERTMISTLSPGNCYCVNSIYINDEKTPISLYKKLFIISIFNSFVFDFLLRRFVDSNVLKSCLYQCPMPQPEEKEILSNPLYLNLAKNTSLLIAKNDPENFKYLLYLEYFKFDKEKVNKILKLDKEDEFFKEKENENNFIIASLYSLAKEDFITLLGDFKALKNKKKGEDYISSLIKGYDNYLLNNKIFYHK

SEQ ID NO: 17 - DNA, 197 bp, Borrelia burgdorferi
ctggctgctacaccacttgttgatgatactgtaattggaaagttgagaactgcaaaaatcaacttttattcacttcttaatgaaactgggcttgatggtgtacccgcatttaaagaaggtgttgacctagctggaggtgcaatagacgaacaatttacataccactatataaaaaatgaagcgattattgagcttat

SEQ ID NO: 18 - protein, 65 aa, Borrelia burgdorferi
LAATPLVDDTVIGKLRTAKINFYSLLNETGLDGVPAFKEGVDLAGGAIDEQFTYHYIKNEAIIEL

SEQ ID NO: 19 - DNA, 1113 bp, Borrelia burgdorferi
ttgccgcaagatacaattagtgtaagtttgcttgactctagaattcaagctagtaggcccaattattataatccacttttggtttacaaaacagctaaaatcaaagttaataaagatgctgctaactataaaatattgaatttaaccgttaataactatgaaaaacaaattgaaactttagaaaaagataatgggaatggacaagatcagtttggaaaagaaaaaacactgcttaaaaccgcaatgtcgaattttttcaattcaagtgaagaatcattaaaatcagccgatctttttatttataaggataaacccgaagagttaaaaaaatatcttaaagtacatagacacacttttgttgtacttattaatactgagggtgataattccgatgatggacttaagatttataaagatgattatgataagtttaaaacaccttcaattttttttgtattctcaactaaagaacaagaaataaaagaactatttaaagataaaggcaatactgaaaaagaaagaaatattgctgtttacagcaataataaagacaatttacacctcaaatttataagtcaatatttacatcaagctagtatttttcatgctgtaaatccttatggcatgccgctggctgctacaccacttgttgatgatactgtaattggaaagttgagaactgcaaaaatcaacttttattcacttcttaatgaaactgggcttgatggtgtacccgcatttaaagaaggtgttgacctagctggaggtgcaatagacgaacaatttacataccactatataaaaaatgaagcgattattgagcttattagaatttggaacaaaaacaataggcaaaatagcaaattatctgcactacaacttagtggggctagagacaatgcatatacttcagcaattgaatgtttactgaaaaggtttgtggatagaggacttattatacagtataaaaatttaagtcttactctttctcctacaccacaacttaaattagaacttagcgtgaatattacttataactttagcattaatgctgtttctttagtaattactactcaagatatagttgattatcaaaacagcttaagtgcttaa

SEQ ID NO: 20 - protein, 370 aa, Borrelia burgdorferi
MPQDTISVSLLDSRIQASRPNYYNPLLVYKTAKIKVNKDAANYKILNLTVNNYEKQIETLEKDNGNGQDQFGKEKTLLKTAMSNFFNSSEESLKSADLFIYKDKPEELKKYLKVHRHTFVVLINTEGDNSDDGLKIYKDDYDKFKTPSIFFVFSTKEQEIKELFKDKGNTEKERNIAVYSNNKDNLHLKFISQYLHQASIFHAVNPYGMPLAATPLVDDTVIGKLRTAKINFYSLLNETGLDGVPAFKEGVDLAGGAIDEQFTYHYIKNEAIIELIRIWNKNNRQNSKLSALQLSGARDNAYTSAIECLLKRFVDRGLIIQYKNLSLTLSPTPQLKLELSVNITYNFSINAVSLVITTQDIVDYQNSLSA

SEQ ID NO: 21 - DNA, 1088 bp, Borrelia burgdorferi
cactatattataattccagttcaatcagaaatatggtcaatagaaagttttaatattttgatcaatattattaatggtattacaagacatagagaaaaaaatataatatttctattatagaaaaccattttctaaaaaatggaaaaattcttaaagaatttttttatacagaattatatcttttttttgttttcttttcttccatttttactatttttctttatttttaggaaaaaccattcgtccactaccatacgattttctctataggtaatattatagttaaagtaattttgaatatatagaaaaaataattttaaggtctatttaatttcttgataatatatttctcttgaagaagattatactcttttagttataagaatttttattctatgtttttatatttactgcaaaaatagtaattagtgtaagtgtacttatccattttagcattaatttattcttaacagtattattattatcaagaatggtctaagaggcaagataatttttaaaaaagcctaagatataagtacttaggcttttttaaaataagaagatattgttgtgctagttatattgatttggcatccttttctaagtcattaagtatagtagcaaggctacctttactatcatcattcatataaattaatgttttgtgtattagtttaatgataagataagcttgagctaccttctctttagtaatcccatcaatagtaacgctagaaagattgctaccaagatggcctttagccttaccacataaagtctttagcttatctttgatgttctttaagtttataaatttagcatcaaactcttgttctgtctgataacccaatttcttacgctgatcaatcttatcttttgcttctataagactactaagctttgtaatctcataaataacatctttaagagattggtttagtgtgtctttagtagaattattgttgtcttcataattttcaatacctttttcaagatcttttaatttatgggaaatgctataagcctcaagcccagccatcttaacaagattagttaagtcagaagtagtttgcatgaatttttgaagt

SEQ ID NO: 22 - protein, 39 aa, Borrelia burgdorferi
HYIIIPVQSEIWSIESFNILINIINGITRHREKNIIFLL

SEQ ID NO: 23 - DNA, 750 bp, Borrelia burgdorferi
atggatattaaaaaaccagatattatagcactcacatcagttaaaggaggagttggaaaaagcacactttctatacttttttcttatttgttgaaggaattgggtaaaaaaatattactaattgatttggatccacagaattctttaacatcatattttaccaagtatattcctgacgccgaaacatataatgtgtatagtatgttaaaaggagatttctattttaaaaaatacttaaataaaattaatgattatatgtatataattccttctcatcctatgttagaaaaatttaatacagaaaccgatcaagaaactttcttggaatattatttaaatagaaatataataaattgcgattttgattatattttgttagacacttctccaggttcaaatttgcttttaaagagtgctttaaatacttcaaattatattataattccagttcaatcagaaatatggtcaatagaaagttttaatattttgatcaatattattaatggcattacaaaacatagagaaaaaaaatataatatttctattgtagaaaaccaatttataaaaaatagaaatattcttaaagaagtagaaaatctgattcataaagaatacaaagaatatatcaagggcaagatacatttttataatagtataaaagttcttataactaaaagactcgaaccttcttcaagagaaatatattatcaagaaattaaagaaactttgaaaaatattttttctttataa

SEQ ID NO: 24 - protein, 248 aa, Borrelia burgdorferi
MDIKKPDIIALTSVKGGVGKSTLSILFSYLLKELGKKILLIDLDPQNSLTSYFTKYIPDAETYNVYSMLKGDFYFKKYLNKINDYMYIIPSHPMLEKFNTETDQETFLEYYLNRNIINCDFDYILLDTSPGSNLLLKSALNTSNYIIIPVQSEIWSIESFNILINIINGITKHREKKYNISIVENQFIKNRNILKEVENLIHKEYKEYIKGKIHFYNSIKVLITKRLEPSSREIYYQEIKETLKNIFS

SEQ ID NO: 25 - DNA, 509 bp, Borrelia burgdorferi
cattatcatttaaatatttctcataaaatcaaatatattacacaagttagtactaatcctgttaagtttattctttttgcaaataaaataaagaattttccaaattcttactataattatttagtaaataatttgcgtaaaattggatataaaaatattccaattttagtagaattaaaggaaaaaataagagatttaaagtgagatatatatttttattcttaatatttaagagtctaaatctttttgcgcttgagagttttttttatgattttgatgtaaggactaaatatttaaaatattttaattcaagctatgcgcaaaaaaaaataagcccaataaaacattttattacagaagattgttacattgaagtttcaagtgaaatttcttctgagtatgtttattattctttttttaataaaaaaaagaatgtaggttatatttttccagggtcttacgtgattaaagtgggcaaagagggtattgagcagataaaaatatttttt

SEQ ID NO: 26 - protein, 67 aa, Borrelia burgdorferi
HYHLNISHKIKYITQVSTNPVKFILFANKIKNFPNSYYNYLVNNLRKIGYKNIPILVELKEKIRDLK

SEQ ID NO: 27 - DNA, 1302 bp, Borrelia burgdorferi
ttgcttagttataaaaaggttcttattgttggtagaccaaatgttggtaaatctgctttatttaatcgaattttagatacaaaaagaagtattactgagagtacttacggtgttactagagatttagttgaagaagtttgtaaggttgattcttttaagtttaaattaatcgatactggtgggtttactatcttgaaagatgagattagcaaaattgttgtgcaaaaggttttaagctctttagaaaaagttgatttaattttattggttttagatattaatgaaattttacttgaagattatcagattattgaaagactgagaaaatatagtagtaaggtggttttggttttaaacaaagtagatactaaggataaggaatgtttagctcacgaatttcataatttaggattcaagcgctattttctggttagtgcagcccattgtcgaggcattactaagcttagagattttttaaaagtagaagttggtgaagttggcattgagagtggcgctgatattaaggttgggattataggcaagcctaattcaggcaaatctacccttattaattatttatctggaaatgaaattgcaattgtttctgatcaacctggtactactagagattttattaaaactaagtttactagaaatgggaaagtttttgaggttgttgatacagctgggataaggcgaagggcaagagtaaatgaaattgttgaatattattctgttaatagggccctaaaagtaattgacatggtagatattgtgtttttattgattgatgttcaagaaaaattgacttctcaggataaaaaaattgctcattatgttactaaaaaggggaaaggaattgttattgtgtttagcaaatgggatcttgtggatgagtctaaaggttattttgaagccttaaagagccatgtgaagtttttttttcctattttaaattttgctcctatatttagaatttctgttcataaaaggataggtttagattctctttttaaagaatcttttaagttaaaagatcagcttgagcttaaaaccagtactccagatctaaataaaatgttaaatttatggatcaaagattatcatttaaatatttctcataaaatcaaatatattacacaagttagtactaatcctgttaagtttattctttttgcaaataaaataaagaattttccaaattcttactataattatttagtaaataatttgcgtaaaattggatataaaaatattccaattttagtagaattaaaggaaaaaataagagatttaaagtga

SEQ ID NO: 28 - protein, 433 aa, Borrelia burgdorferi
MLSYKKVLIVGRPNVGKSALFNRILDTKRSITESTYGVTRDLVEEVCKVDSFKFKLIDTGGFTILKDEISKIVVQKVLSSLEKVDLILLVLDINEILLEDYQIIERLRKYSSKVVLVLNKVDTKDKECLAHEFHNLGFKRYFLVSAAHCRGITKLRDFLKVEVGEVGIESGADIKVGIIGKPNSGKSTLINYLSGNEIAIVSDQPGTTRDFIKTKFTRNGKVFEVVDTAGIRRRARVNEIVEYYSVNRALKVIDMVDIVFLLIDVQEKLTSQDKKIAHYVTKKGKGIVIVFSKWDLVDESKGYFEALKSHVKFFFPILNFAPIFRISVHKRIGLDSLFKESFKLKDQLELKTSTPDLNKMLNLWIKDYHLNISHKIKYITQVSTNPVKFILFANKIKNFPNSYYNYLVNNLRKIGYKNIPILVELKEKIRDLK

SEQ ID NO: 29 - DNA, 924 bp, Borrelia burgdorferi
cacgtagattttacaattgaagttgaaagatctcttagagtgcttgacggggcaatattggttcttgattctgttgcaggagttcaatcccaatcaataactgttgatcgacagcttaaaagatatagcgtgccgcgccttgcatttgtaaacaagtgtgataaaaccggagcaaatccctacaatgtaaaagatcaactaagatcaaaacttgacttaaactccgttttaatgcaaattccaattggattagaagacaaacatattggagttatagaccttgtattaatgaaagcctactattttgaaggaaaagatggaacagaaataatagaaaaagaaataccctcagatctcttagaagaagcaaaaagcaaacgagaaataatgcttgatactcttgctgactttaatgatgaacttatggaattacacatggaaggaaaagaagttcctactgaaataatatacaatgcaactagaacaggaacattggctttaaaattatgccctgtatttatgggatctgcttataaaaacaaaggagtaaatatattgaatttggagcatctcctagagcttctcttagtttattaaagtgtgctcgtgttaatgctctttatgaggggagaatatttgttttaccagaagatgtcaaagctgttgcttacagcgtattaaggcatagaattacaccatcttatgaggcagaggtagaggaaatgagcattgatgatattattagaatgcttctttctgctgtagcgcttccttaggaaatgattttaaaatgatacaagataatgagataagtagtagtactaaaactaggataaaagctttaaaattcttttcaaggaaaatgctttcagagcttaattttggtggttatcgttcaatttttaaaggtcttggccttgaatt

SEQ ID NO: 30 - protein, 198 aa, Borrelia burgdorferi
HVDFTIEVERSLRVLDGAILVLDSVAGVQSQSITVDRQLKRYSVPRLAFVNKCDKTGANPYNVKDQLRSKLDLNSVLMQIPIGLEDKHIGVIDLVLMKAYYFEGKDGTEIIEKEIPSDLLEEAKSKREIMLDTLADFNDELMELHMEGKEVPTEIIYNATRTGTLALKLCPVFMGSAYKNKGVNILNLEHLLELLLVY

SEQ ID NO: 31 - DNA, 2082 bp, Borrelia burgdorferi
atggactataataaattacgaaacataggtattagcgcacacatcgactcaggaaaaaccactcttacagaacgtattcttttttattgtaataaaattcatgcaattcacgaagtaaaaggcaaagatggggttggtgcaacaatggactcaatggaacttgaaagagaaagaggaatcacaatagcatcagctgcaactcacgttgaatggaaagattttccgataaatattattgatacacccggacacgtagattttacaattgaagttgaaagatctcttagagtgcttgacggggcaatattggttcttgattctgttgcaggagttcaatcccaatcaataactgttgatcgacagcttaaaagatatagcgtgccgcgccttgcatttgtaaacaagtgtgataaaaccggagcaaatccctacaatgtaaaagatcaactaagatcaaaacttgacttaaactccgttttaatgcaaattccaattggattagaagacaaacatattggagttatagaccttgtattaatgaaagcctactattttgaaggaaaagatggaacagaaataatagaaaaagaaataccctcagatctcttagaagaagcaaaaagcaaacgagaaataatgcttgatactcttgctgactttaatgatgaacttatggaattacacatggaaggaaaagaagttcctactgaaataatatacaatgcaactagaacaggaacattggctttaaaattatgccctgtatttatgggatctgcttataaaaacaaaggagtgcaattgctcttagatgctgtaaccagatttttgccatcccctcatgatataaaaaacaccgctcttgacctaaataataatgaaaaagaaatcgatcttaaaattgacaacgagctcccaactgttgctcttgcatttaaacttgaagacggacaatacggacagttgacttatgtgagaatctatcaaggaattttaaaaaaaggacaagaacttatcaactcaagaacttctaaaaaattcaaagttggaaggcttatcagaatgcatgccaataatacagaagacattgaatttggaggaagtggtgacattgttgctttatttggaatagaatgtgcatcaggagatacgttttgtgatccatcgatcaactattcaatgacatcaatgtttattccagatccagtaatttctctttctgtaaaaccaaaggataaaaaatctgctgataatatggccaaagcccttggaagatttacaaaagaagatccaacatttaaaacttatgttgacattgaatcaaatgaaacaataattcaaggaatgggagagctacacttagaagtttacattgaaagaatgaaaagagagttcaaggcagaagttgaaaccggaatgccgcaagtagcctatagagaaacgattacaagaaaagctgaatttaattatactcacaaaaagcaatctggaggagctggtcagtttggacgagttgcagggtttatggaacctcttgacaaagaaggagaaacatacgaatttgtcaatctaataaaaggaggagtaatcccaacagaatatatcccatcatgtgataaagggttccaaaaagcaatggaaaggggaacattaattggctttccaatagttgacataaaaattacaatcaatgatggccaatatcacattgttgactcatctgatattgcattccaattagcagcaattggagcttttagagaggcttatgaaaaagcaaagcctacaatccttgagccaataatgaaagttacccttgaaggacctactgaattccaaggcaatatgtttggacttttaaatcaaagaagaggaataataacaggttccctagaagatggaagtttttcaaaagttgaggctgaggtgcctttaagcgaaatgtttggattttcaacagtccttagatcctctacccaaggaaaagcagaattctcaatggaattcttaaggtatggaaaagttccaagcactatatttgatgaacttcgcaaaaaatttaacgatcaaaacaaatcttaa

SEQ ID NO: 32 - protein, 693 aa, Borrelia burgdorferi
MDYNKLRNIGISAHIDSGKTTLTERILFYCNKIHAIHEVKGKDGVGATMDSMELERERGITIASAATHVEWKDFPINIIDTPGHVDFTIEVERSLRVLDGAILVLDSVAGVQSQSITVDRQLKRYSVPRLAFVNKCDKTGANPYNVKDQLRSKLDLNSVLMQIPIGLEDKHIGVIDLVLMKAYYFEGKDGTEIIEKEIPSDLLEEAKSKREIMLDTLADFNDELMELHMEGKEVPTEIIYNATRTGTLALKLCPVFMGSAYKNKGVQLLLDAVTRFLPSPHDIKNTALDLNNNEKEIDLKIDNELPTVALAFKLEDGQYGQLTYVRIYQGILKKGQELINSRTSKKFKVGRLIRMHANNTEDIEFGGSGDIVALFGIECASGDTFCDPSINYSMTSMFIPDPVISLSVKPKDKKSADNMAKALGRFTKEDPTFKTYVDIESNETIIQGMGELHLEVYIERMKREFKAEVETGMPQVAYRETITRKAEFNYTHKKQSGGAGQFGRVAGFMEPLDKEGETYEFVNLIKGGVIPTEYIPSCDKGFQKAMERGTLIGFPIVDIKITINDGQYHIVDSSDIAFQLAAIGAFREAYEKAKPTILEPIMKVTLEGPTEFQGNMFGLLNQRRGIITGSLEDGSFSKVEAEVPLSEMFGFSTVLRSSTQGKAEFSMEFLRYGKVPSTIFDELRKKFNDQNKS

SEQ ID NO: 33 - DNA, 908 bp, Borrelia burgdorferi
ccagcattgtctacaatagagtatctaaaagaaagaggagctaggattgtgcttgtaagtcatttgggtagaccagatggaaagaaaaatcctaaatattctcttaagccggttgccaataggttgtcagagcttttaggccaagatgtcaagatgctttctgattgtattggaagtgaggttgtaaatagtaccttgcaaatgaaggatggggatgttgtattgcttgaaaatgtaaggttttatgcagaagaagagaaaaacgataaaaattttgcaaaaaaactttctgaaaatggagatgtttttgtaaatgatgcttttggcgcagctcatagagctcatgcttcaactgtgggagttgctgattatttgccgtctgttggtggatttttaatggaaaaagaagataaatttcttggtggaatattaaaaaatcccgaaaggccatttgtttcaattattggtggttctaaggtttcttcaaagattgcggtactagaatcacttttgtcaaagtctaatgtagttgtgattggcggtggaatggcttatacttttttgcattccgaaggatattctataggcaaatctcttttagaggacgaatatattggtatagcatcttcttttttaaagaaagcaaaagagctgggcgttaaagtaattttaccacttgatcatattgtagctgatgattttaacaaaaattcaattcctgagtatattgactcctttaatattcctgaaaacaagattggcatggatattggagctaatactttaaaagaaattgaaaatgttgtgaaaactgccaaaactataatttggaatggtcctcttggtgtatttgaatttgattcattttcaaaagggactgctaaggttgccgaaatggtagcatcttg

SEQ ID NO: 34 - protein, 302 aa, Borrelia burgdorferi
PALSTIEYLKERGARIVLVSHLGRPDGKKNPKYSLKPVANRLSELLGQDVKMLSDCIGSEVVNSTLQMKDGDVVLLENVRFYAEEEKNDKNFAKKLSENGDVFVNDAFGAAHRAHASTVGVADYLPSVGGFLMEKEDKFLGGILKNPERPFVSIIGGSKVSSKIAVLESLLSKSNVVVIGGGMAYTFLHSEGYSIGKSLLEDEYIGIASSFLKKAKELGVKVILPLDHIVADDFNKNSIPEYIDSFNIPENKIGMDIGANTLKEIENVVKTAKTIIWNGPLGVFEFDSFSKGTAKVAEMVAS

SEQ ID NO: 35 - DNA, 1182 bp, Borrelia burgdorferi
atgtcaataaaaacagtaaaagactttagtagctttgcgggcaagcgagctttagtaagatgtgattttaatgttcccctaaaagaggggagcattagcgatgatactagaattagggcagcattgtctacaatagagtatctaaaagaaagaggagctaggattgtgcttgtaagtcatttgggtagaccagatggaaagaaaaatcctaaatattctcttaagccggttgccaataggttgtcagagcttttaggccaagatgtcaagatgctttctgattgtattggaagtgaggttgtaaatagtaccttgcaaatgaaggatggggatgttgtattgcttgaaaatgtaaggttttatgcagaagaagagaaaaacgataaaaattttgcaaaaaaactttctgaaaatggagatgtttttgtaaatgatgcttttggcgcagctcatagagctcatgcttcaactgtgggagttgctgattatttgccgtctgttggtggatttttaatggaaaaagaagataaatttcttggtggaatattaaaaaatcccgaaaggccatttgtttcaattattggtggttctaaggtttcttcaaagattgcggtactagaatcacttttgtcaaagtctaatgtagttgtgattggcggtggaatggcttatacttttttgcattccgaaggatattctataggcaaatctcttttagaggacgaatatattggtatagcatcttcttttttaaagaaagcaaaagagctgggcgttaaagtaattttaccacttgatcatattgtagctgatgattttaacaaaaattcaattcctgagtatattgactcctttaatattcctgaaaacaagattggcatggatattggagctaatactttaaaagaaattgaaaatgttgtgaaaactgccaaaactataatttggaatggtcctcttggtgtatttgaatttgattcattttcaaaagggactgctaaggttgccgaaatggtagcatcttgctcaggtttaaccgttgttggtggtggagattctgttgcagctgttaataaatttaatttatctgataagattactcatgtttcaacgggcggtggtgcatcgcttgaatatcttgaagggagaattttgccgggtattaaggttttggagaattaa

SEQ ID NO: 36 - protein, 393 aa, Borrelia burgdorferi
MSIKTVKDFSSFAGKRALVRCDFNVPLKEGSISDDTRIRAALSTIEYLKERGARIVLVSHLGRPDGKKNPKYSLKPVANRLSELLGQDVKMLSDCIGSEVVNSTLQMKDGDVVLLENVRFYAEEEKNDKNFAKKLSENGDVFVNDAFGAAHRAHASTVGVADYLPSVGGFLMEKEDKFLGGILKNPERPFVSIIGGSKVSSKIAVLESLLSKSNVVVIGGGMAYTFLHSEGYSIGKSLLEDEYIGIASSFLKKAKELGVKVILPLDHIVADDFNKNSIPEYIDSFNIPENKIGMDIGANTLKEIENVVKTAKTIIWNGPLGVFEFDSFSKGTAKVAEMVASCSGLTVVGGGDSVAAVNKFNLSDKITHVSTGGGASLEYLEGRILPGIKVLEN

SEQ ID NO: 37 - DNA, 645 bp, Borrelia burgdorferi
cactccaataattttgatgatatcaagaaaattgaatctgatttaatcttgtattttgagaaaaatagtgattttaagagtgtaaactattgggcggagattataaaaaactatttcaagaaaaataatagattaaaggatttacaagattttgaaaagtttgtgtcgtttaagaggactgcttatggccctagtccattaatattctttagtatcttaaaagaatacgaacggtttgatgagatatttgcagcatagcaagattcttacatgttaaagcccccattatgggggctgctagattaattgcaggtagcagttgcttgatctgcaaaattatctatattgccgccgctaaaaaacccttgaacggtagttttgaaggtgttttttccttcatcatttccattacatttatcaagttcagtctttatatgttcaagtgctgatttaattttgccttcatcattttctaagaatttatcaaattttccaacaccagttaaagcggtttttaaccagtcaagttgtgttttttgatcatcagatagcttttctctaagcaggtcttctttagatttagatttaggtttttcttgtgttgcttctttttgggttaaatcacgcttttgtctgctttttgtc

SEQ ID NO: 38 - protein, 85 aa, Borrelia burgdorferi
HSNNFDDIKKIESDLILYFEKNSDFKSVNYWAEIIKNYFKKNNRLKDLQDFEKFVSFKRTAYGPSPLIFFSILKEYERFDEIFAA

SEQ ID NO: 39 - DNA, 1059 bp, Borrelia burgdorferi
ttgctatatacttatatttattactataaaaggagtaaaaagatggaaaatctttcaaacaataataatcaagaaatacaaaataatattcaagcaaaaataagcttcagaaaagatatgaaaaccctaaaaatgaatttaccagggattgacaaaagtcttaaaggatatggatataaatatcaaaatttcaatgaaatagttagagaaattaaaaatgttattaaaaagcacaatttggagcttgatattgagcaatatccaatttctatagagggtcaatatggcatagttgattatattaggactacattctacagtacaagtactggatatgaattttcttttgatacgcgaattcctacagaaaatttacaatggaacaatgaaaatgggtctaaagttacaaatacagtgtatcagatgtttggttcaggcattacttatgtcaaaaggtatgctttagttgcagctcttggtatagaaagtgaaatagatactgatgcagctcctatttacaataatcacgaaaacgaaaattctatgcctagcaaacaatctagtgttaatcaaaagcaagaacaaaaaagagaacaaaaacaagagattaatcaaattcaaaaaaataacactattcaaaaccagaaaagagatattaaacaagaacaaaaaaaagataggctttattattacggtgtttttaaagaagctttgtctaatataaaagattgggtaaatagccctacaacaaaagataatataaactcaattattcaaaaaataagctttattcagaatatagactccaataattttgatgatatcaagaaaattgaatctgatttaatcttgtattttgagaaaaatagtgattttaagagtgtaaactattgggcggagattataaaaaactatttcaagaaaaataatagattaaaggatttacaagattttgaaaagtttgtgtcgtttaagaggactgcttatggccctagtccattaatattctttagtatcttaaaagaatacgaacggtttgatgagatatttgcagcatag

SEQ ID NO: 40 - protein, 352 aa, Borrelia burgdorferi
MLYTYIYYYKRSKKMENLSNNNNQEIQNNIQAKISFRKDMKTLKMNLPGIDKSLKGYGYKYQNFNEIVREIKNVIKKHNLELDIEQYPISIEGQYGIVDYIRTTFYSTSTGYEFSFDTRIPTENLQWNNENGSKVTNTVYQMFGSGITYVKRYALVAALGIESEIDTDAAPIYNNHENENSMPSKQSSVNQKQEQKREQKQEINQIQKNNTIQNQKRDIKQEQKKDRLYYYGVFKEALSNIKDWVNSPTTKDNINSIIQKISFIQNIDSNNFDDIKKIESDLILYFEKNSDFKSVNYWAEIIKNYFKKNNRLKDLQDFEKFVSFKRTAYGPSPLIFFSILKEYERFDEIFAA

SEQ ID NO: 41 - DNA, 241 bp, Borrelia burgdorferi
ctgctgggaatatggaagaaagcagaaaattatgttgacaagtttgatgaacttataattgtcaagaataaagaattatttgttatttaaattgaaagatatgattctaatagaaaatattatcctttaattagatttgcaaatttaattgaatttgaaatttttaaaatttaattgcagaaaaactttttactgacaatcaagttttaaacaatttcaacaaacaagcaagctttgatta

SEQ ID NO: 42 - protein, 29 aa, Borrelia burgdorferi
LLGIWKKAENYVDKFDELIIVKNKELFVI

SEQ ID NO: 43 - DNA, 93 bp, Borrelia burgdorferi
ttggtgctgggaatatggaagaaagcagaaaattatgttgacaagtttgatgaacttataattgtcaagaataaagaattatttgttatttaa

SEQ ID NO: 44 - protein, 30 aa, Borrelia burgdorferi
MVLGIWKKAENYVDKFDELIIVKNKELFVI

SEQ ID NO: 45 - DNA, 356 bp, Borrelia burgdorferi
catacttgtggatctgataaaatggattctactagtgtagacatttttgatgatttttatcctttaaaaattgagtcgttgcaaggtaaaaatttagctgtaatcaaagagcttagcgaagatctaatggacaaaaatgttgcaaatagttttgcaaagtttaaattagaccttttgtcaaagggtattaatataaaagaagtttcaatagaagagattaattttattttatcaatttattatataatttctcctgttgaagcatcctccaatcttgctcgttatactggactttgttacggcaagagaatctctgaaggtttgagtcttaatgatttttattttaaacataggag

SEQ ID NO: 46 - protein, 118 aa, Borrelia burgdorferi
HTCGSDKMDSTSVDIFDDFYPLKIESLQGKNLAVIKELSEDLMDKNVANSFAKFKLDLLSKGINIKEVSIEEINFILSIYYIISPVEASSNLARYTGLCYGKRISEGLSLNDFYFKHR

SEQ ID NO: 47 - DNA, 1491 bp, Borrelia burgdorferi
atggatatttttcatctcctaaaatattggagtaaggataaggtgttggacttaagtaatttaactttaaccaaaattcaagaattagttttaactagaaaatgtaaaatttatgatattttgcttgcttataaaaataattatgagctaaataaagatatcaatggatatattgaattttttgatgattctttagagattgcaaaaaggtatgacgattgtttaaaaaattgtgaattagaagatttgcctttaattggtatgcttattgcagtcaaagataatatttcaattcaagataaatctttaacttgtgcttctgagattttaaaaggttatatttctccttatgatgcgactgttattaaaaggcttaagaataaaggagcaattttaattggtagaaccaatatggatgaatttgccatgggttctacttgtgaattttcttattacggtgcaactttaaatcctttaaatagagaatatgttataggtggtagttctggaggctctgcagctgtagttgcagcttttcaagcacctttttcgcttggtagtgatactggaggttctgttaggctgcccgcatctttttcaggaattttgggttttaaaccttcttatggaggtctttctcgctatgggcttgcatcttatgcttcgtcttttgatcaaataggatttttttctcattctattgaagatattgctttaattttaaagcatacttgtggatctgataaaatggattctactagtgtagacatttttgatgatttttatcctttaaaaattgagtcgttgcaaggtaaaaatttagctgtaatcaaagagcttagcgaagatctaatggacaaaaatgttgcaaatagttttgcaaagtttaaattagaccttttgtcaaagggtattaatataaaagaagtttcaatagaagagattaattttattttatcaatttattatataatttctcctgttgaagcatcctccaatcttgctcgttatactggactttgttacggcaagagaatctctgaaggtttgagtcttaatgatttttattttaaacataggagcaatttcttgtcagaagaagttaaaaggcgtattgttcttggaaattatttgttatcagaaaggtatgattctaaatattatgcaaaagcttgtgaaattcttcaaaatttgattattcctaaatttaacaagctttttgaaagctgtgattttattattaccccaacaagctttgttaaaccttttagacttggtttggattttgatgatcctgttaaaatgtattattcagatatttgtactgttattgcaaatcttattggagcccctgctatttcgcttccatattctaaggatgaggaaggattgtcaattgggatgcaaattattgggcgtagcaaaaaggattttgaacttttaagtttttcaaaaaatgtgattagggaattaggattgaatggaatataa

SEQ ID NO: 48 - protein, 496 aa, Borrelia burgdorferi
MDIFHLLKYWSKDKVLDLSNLTLTKIQELVLTRKCKIYDILLAYKNNYELNKDINGYIEFFDDSLEIAKRYDDCLKNCELEDLPLIGMLIAVKDNISIQDKSLTCASEILKGYISPYDATVIKRLKNKGAILIGRTNMDEFAMGSTCEFSYYGATLNPLNREYVIGGSSGGSAAVVAAFQAPFSLGSDTGGSVRLPASFSGILGFKPSYGGLSRYGLASYASSFDQIGFFSHSIEDIALILKHTCGSDKMDSTSVDIFDDFYPLKIESLQGKNLAVIKELSEDLMDKNVANSFAKFKLDLLSKGINIKEVSIEEINFILSIYYIISPVEASSNLARYTGLCYGKRISEGLSLNDFYFKHRSNFLSEEVKRRIVLGNYLLSERYDSKYYAKACEILQNLIIPKFNKLFESCDFIITPTSFVKPFRLGLDFDDPVKMYYSDICTVIANLIGAPAISLPYSKDEEGLSIGMQIIGRSKKDFELLSFSKNVIRELGLNGI

SEQ ID NO: 49 - DNA, 436 bp, Borrelia burgdorferi
ctatacaatcctttagcaaaagaactttcacttgtccatctaataatagaaaaaactaaactaaaagaccttattgatgatgatgctagcgaagcaatacttgacaaagtccccgctgtaatcattgattctggagaagatgtagaaactattttgcaagccttgttgtatgaattcggatacgcatacactttctcttcagtcggcaaactacaaatacttccaatttggaaaagcgaagttattactaaagaaattgaaatttgtactgtggatgctgaaagttatgttatgtcaaaaagtagctccagtagctacgattcaactaaagttatttggagagaaggcaaattccaaagcaaagaagaagcaatgtccaaaaaacgaccactgtactcagccccaatcaatgttgctggtaccgatggtgcactgt

SEQ ID NO: 50 - protein, 145 aa, Borrelia burgdorferi
LYNPLAKELSLVHLIIEKTKLKDLIDDDASEAILDKVPAVIIDSGEDVETILQALLYEFGYAYTFSSVGKLQILPIWKSEVITKEIEICTVDAESYVMSKSSSSSYDSTKVIWREGKFQSKEEAMSKKRPLYSAPINVAGTDGAL

SEQ ID NO: 51 - DNA, 2691 bp, Borrelia burgdorferi
ttgtatttgggtgttacaaaaaatacagcagaagataaggattgtattaagttgaaagatcaagacaattcaagtcaagaatccgaagccttaaatcaagaaaccgaaaaaggtgccgaaactgaagaaatctcaaatcccaaagaagatacatcggaaaattgtacttctttggggcttgatattgtattctacacggacgagggtcaaatttttgaacttaccccttttgttgacactacaagcattgttctagaagaaaaaatagttgatccaagcctaaaaacagcagcaagcacgttcagctttagtgtaagcggcctatcagaagaatttttgaatttcttattcttcagaaacgaagacattttcgtaaaagtaaatgataagaataaccccgtttttaggggaattctagaaaaatttttcagcagagatctgttcaatagcaccaaaagtgtatcattcaccgttaacgactactctagcctactaaagattgcattcgaaaatccagttcaattcccaatcaattttttaccagactggctttttgtatacaatcctttagcaaaagaactttcacttgtccatctaataatagaaaaaactaaactaaaagaccttattgatgatgatgctagcgaagcaatacttgacaaagtccccgctgtaatcattgattctggagaagatgtagaaactattttgcaagccttgttgtatgaattcggatacgcatacactttctcttcagtcggcaaactacaaatacttccaatttggaaaagcgaagttattactaaagaaattgaaatttgtactgtggatgctgaaagttatgttatgtcaaaaagtagctccagtagctacgattcaactaaagttatttggagagaaggcaaattccaaagcaaagaagaagcaatgtccaaaaaacgaccactgtactcagccccaatcaatgttgctggtaccgatggtgcactgtacgttgcagttcttcaaaaaggcgtagtgtaccccgactttgcagacaaggtcggaagcagtgtttttcaagaatacgatcctagctggttagatacagtttacaaatgggacttcaaaaggcgcgaggtttggcacgaccactacgccataaatgaacatttagcaataatctcaactcataaccttgaagtaaggtttagtgccgactcctccatcaagcttgaaaataaagaatattttccaacaaaggctaagctttggtttagaaacacttctagtagtcaaggctccaaatatatttaccactttgacatttatggtgatgttttctacacaatagctcaaaatgtactccaaaccgacaatgctgatgatttttactctaaaaagtttgaatacacaaccagattcattttttccgaagattcagcattaagactttttgaattcctaacaaacttacgtgtcaaaggacacactattgttaattttaggtcaatgcagaatctaaatataaccgattttgtcaaacttagacttgacaatattgggattgatcattttttccttatactttcaaaaaaaattactaatttcgatctggatatcaaactttacgagtacgagggaatcacttggggtgactacactcattacgaataccttacaacttcaaccaaaataggaataagcgactatttgtctggggctcacaaagttgttattgcaccgtttaattatgatggggccgaaacttaccaatttaaagcctccgggtttaaagacgaaaatactttaaatggttcaattgaatttgcaaaacaagctggacttaacaagatcaaactacttaaaggcgatttttacatttatgatcaagtaaaactaaacaatttagaaatcgaaggcgatgacgaagttttcataagaagcacggggttttctaaacatatttttacatcagaaaaatcattcaggctttctggagtcaatatatgtcaacaaccaatgagtgaactttacattaacggaggagatctaaaccaacaagaactggcaccttacctagaagacaaaagctttacacagcccgctgttgtagtgctctgctcatcatattcattgcaagaagaggccagatcatcagtctatgttacagatgcaagctttgtgtacatcaaaaatgtcacatttacgtgcgcattaaacaaagcattgcatttcaaaaaaacaaaaaaaattttacttgaagatgtaaattttgattctacaaatcaaagttttgacatagaaaatgttaccaatctaacactaattaatgttaatgctcaaggaagcaaaactgcatcagttgcaaatggacttaatgcaataattagagaaagcttctttaccaacaacaccaatgcgcttaagctttccaacttttcaagtttgaaaataaccgacacacagtttatcaaaaatagcggtactgcgctgcatttacaagatattactagcgcaagactaagtaacaatacgtttacagaaaataaagttggacttgaaaatcatgcttttgacttgatcgtacgcgatacttttgtaaaaaacacacttgcccttaatttagcaagaaaatcaaatgcaggggtgcgaacacttgatcttagcacttacatggaaaacactaaagacaaacaggaggttgcttag

SEQ ID NO: 52 - protein, 896 aa, Borrelia burgdorferi
MYLGVTKNTAEDKDCIKLKDQDNSSQESEALNQETEKGAETEEISNPKEDTSENCTSLGLDIVFYTDEGQIFELTPFVDTTSIVLEEKIVDPSLKTAASTFSFSVSGLSEEFLNFLFFRNEDIFVKVNDKNNPVFRGILEKFFSRDLFNSTKSVSFTVNDYSSLLKIAFENPVQFPINFLPDWLFVYNPLAKELSLVHLIIEKTKLKDLIDDDASEAILDKVPAVIIDSGEDVETILQALLYEFGYAYTFSSVGKLQILPIWKSEVITKEIEICTVDAESYVMSKSSSSSYDSTKVIWREGKFQSKEEAMSKKRPLYSAPINVAGTDGALYVAVLQKGVVYPDFADKVGSSVFQEYDPSWLDTVYKWDFKRREVWHDHYAINEHLAIISTHNLEVRFSADSSIKLENKEYFPTKAKLWFRNTSSSQGSKYIYHFDIYGDVFYTIAQNVLQTDNADDFYSKKFEYTTRFIFSEDSALRLFEFLTNLRVKGHTIVNFRSMQNLNITDFVKLRLDNIGIDHFFLILSKKITNFDLDIKLYEYEGITWGDYTHYEYLTTSTKIGISDYLSGAHKVVIAPFNYDGAETYQFKASGFKDENTLNGSIEFAKQAGLNKIKLLKGDFYIYDQVKLNNLEIEGDDEVFIRSTGFSKHIFTSEKSFRLSGVNICQQPMSELYINGGDLNQQELAPYLEDKSFTQPAVVVLCSSYSLQEEARSSVYVTDASFVYIKNVTFTCALNKALHFKKTKKILLEDVNFDSTNQSFDIENVTNLTLINVNAQGSKTASVANGLNAIIRESFFTNNTNALKLSNFSSLKITDTQFIKNSGTALHLQDITSARLSNNTFTENKVGLENHAFDLIVRDTFVKNTLALNLARKSNAGVRTLDLSTYMENTKDKQEVA

SEQ ID NO: 53 - DNA, 526 bp, Borrelia burgdorferi
caaataaaaatcgatacattaaacataccgataacaagagatatttatagtctcaccatattaaagaaaaagtcaatatacgacaaaattaaagcattgtttatatcaagctttgcaccattgcaaatggaaaatattattttttcattcttaaagaaagaaaaaataaataatagcggcaaattaaaaatatttaattatacagacaaagaagtagaagaagaaatacataaggttcaaaaagaaattaaaagcaaaattctaaatgaaagtctaacaaatgaagcaatatacactgaaaacaatttaaaagacatatactttgctatcatactatcactatcacctagcctgatattttctttcctaaataacacaaaattcatgacagacaccttgctgttaatatttattagtgtggcaatctatatacctataatgctaaaaattaattacaaatgtttgtctttttttgtttacgccgccttaatgataagcattatattgcccttggatttagaaatta

SEQ ID NO: 54 - protein, 175 aa, Borrelia burgdorferi
QIKIDTLNIPITRDIYSLTILKKKSIYDKIKALFISSFAPLQMENIIFSFLKKEKINNSGKLKIFNYTDKEVEEEIHKVQKEIKSKILNESLTNEAIYTENNLKDIYFAIILSLSPSLIFSFLNNTKFMTDTLLLIFISVAIYIPIMLKINYKCLSFFVYAALMISIILPLDLEI

SEQ ID NO: 55 - DNA, 2322 bp, Borrelia burgdorferi
atgctagacttagaaaaagtaaaaacaatttttaaaaaatacaataaatacgaaataaatattaacgagattcaaatacctgaatatgccctaataccccttgaaacagaaaattcaaaatccataatatacataattgaaaaacaaaaaatagaagaaaatcaaattttatccaaaaactcaaacatagagctatacacatattctccaatatctggaatagtagaaaaaatatacacagccaattttccagatggaaaaaatttaaaatcagcattaatcaaatttcaaggaaaaataaaaactgaaaaaacaccaattgaagaaggagcctcaagagaaaaaaccttggaaaaattaattcaactgggaattccttggtttaacgaaaattctttatttcaattcataaacaaatgtaaaaaaatagacaaaatgattttattaacaaacggaaaagatgcatttacaaatatttcagaagcactgatggcagaaaaattagaagaaattttttcaggattccaaataatcgacaaaatattcaaattcaaagaaataataataataatatcaaataaagacaaactaaaaaaagaattcgaaaaattgagtatttttaaaaacagaaaaataaaaattaaatctttagaaaatgcatatccttatacaaatcacgaaatgataatgcactttttatacaataataaaaatacaaaagatgatataaatccccacaacaatattttattagcaaatattgaagatctatacaatgcaaatcttgtcataaaaaataacaacccttataaagaaaaatttgtagctataaatggaaataaaaaaataaaaagtagaatactgaaggtaaaaattggaacctctttcagccagctaataaatgagaaaattgacacaaaaaaatatgaaatttttttaaacaacccagccaataaaataaaaatcgatacattaaacataccgataacaagagatatttatagtctcaccatattaaagaaaaagtcaatatacgacaaaattaaagcattgtttatatcaagctttgcaccattgcaaatggaaaatattattttttcattcttaaagaaagaaaaaataaataatagcggcaaattaaaaatatttaattatacagacaaagaagtagaagaagaaatacataaggttcaaaaagaaattaaaagcaaaattctaaatgaaagtctaacaaatgaagcaatatacactgaaaacaatttaaaagacatatactttgctatcatactatcactatcacctagcctgatattttctttcctaaataacacaaaattcatgacagacaccttgctgttaatatttattagtgtggcaatctatatacctataatgctaaaaattaattacaaatgtttgtctttttttgtttacgccgccttaatgataagcattatattgcccttggatttagaaattacactaaaaataatttccttattatttacttttttggtattcttttactttttcagactatcagcatttttggcaaaccctatattaatttcttttatgtttttggtattaaattttccggtaagttttaagcaagcctatcaagaggaacttaaaaattcaaattcaataattccaacctggaatgaaataattgaaacaaatccaaacataaaaaatttaaaaagtttaaatacctttacaagatatgaaagtaaaaaaatagatgcaattgaaaactttgtaaatcaaaacattttttccgcttttaatataattgtcacaagatttcatattgaaagtcttttgggagttaataatgaaaaaaaaatatctcctatcttgctttattttggcctcttattaatagttggcaaatatataatcaacaaattaataccactctcattttatataagcttaatggcaatctcgtacatagcacataatataggaatgctaaaccacatcagctccgacatgttaacactacttatttctccaatcccaatgctgttaatatttacaatggcaacagaattgcaaataacacctcactttaaatttgagcaaatactctacggatcaatattggcatttatatactttttgacattaagctacattccttttgaaactttatcagtcataatatctattttcatcttacaaacaagctcaaacttaataaaaaaatatagcttaaccttcaaaattaaaaaaataatgcatgttttgaaaacaaataaagaaaaagcactcaaaacacctcttgagaatgaaaaggatataataaaattatga

SEQ ID NO: 56 - protein, 773 aa, Borrelia burgdorferi
MLDLEKVKTIFKKYNKYEININEIQIPEYALIPLETENSKSIIYIIEKQKIEENQILSKNSNIELYTYSPISGIVEKIYTANFPDGKNLKSALIKFQGKIKTEKTPIEEGASREKTLEKLIQLGIPWFNENSLFQFINKCKKIDKMILLTNGKDAFTNISEALMAEKLEEIFSGFQIIDKIFKFKEIIIIISNKDKLKKEFEKLSIFKNRKIKIKSLENAYPYTNHEMIMHFLYNNKNTKDDINPHNNILLANIEDLYNANLVIKNNNPYKEKFVAINGNKKIKSRILKVKIGTSFSQLINEKIDTKKYEIFLNNPANKIKIDTLNIPITRDIYSLTILKKKSIYDKIKALFISSFAPLQMENIIFSFLKKEKINNSGKLKIFNYTDKEVEEEIHKVQKEIKSKILNESLTNEAIYTENNLKDIYFAIILSLSPSLIFSFLNNTKFMTDTLLLIFISVAIYIPIMLKINYKCLSFFVYAALMISIILPLDLEITLKIISLLFTFLVFFYFFRLSAFLANPILISFMFLVLNFPVSFKQAYQEELKNSNSIIPTWNEIIETNPNIKNLKSLNTFTRYESKKIDAIENFVNQNIFSAFNIIVTRFHIESLLGVNNEKKISPILLYFGLLLIVGKYIINKLIPLSFYISLMAISYIAHNIGMLNHISSDMLTLLISPIPMLLIFTMATELQITPHFKFEQILYGSILAFIYFLTLSYIPFETLSVIISIFILQTSSNLIKKYSLTFKIKKIMHVLKTNKEKALKTPLENEKDIIKL

SEQ ID NO: 57 - DNA, 628 bp, Borrelia burgdorferi
cttttaaaaatatctagctctttaataatagaaaaagaaaaaattgccattattcttgaatcaaacattttcaatacggggcaaaaaataaacaaagcctttgaatcttcaaaaacaaaagctattgactcttttgttgagattgagaaaaatagtcacgtagttcacataaaccatggaattggtatatttaggcaaataaagagaataaaaacaagctctcttgaaaaggattatattgaaattgaatacgctgaaggagaaaaactatttattccaattgaacaaacaaatttaatccaaaaatacattggaagtgatcctaaaaatatcaaattagataaaataagttctaaaacatggataaaaaacaaagcaaacgcaaaaaaaagaatcgaggagattgcagacaaattaatagaactttattcaaaaagagaaagcattaagggtattaaatacccagaagataatgaattacaattgttgtttgaatctgaatttccatacgatgaaactccagatcaaataagagcaataaaagaaataaaagaagatatgatgagctttaaagtaatggatcgccttctttgtggagatgttggatttggaaaaactgaagttgcca

SEQ ID NO: 58 - protein, 209 aa, Borrelia burgdorferi
LLKISSSLIIEKEKIAIILESNIFNTGQKINKAFESSKTKAIDSFVEIEKNSHVVHINHGIGIFRQIKRIKTSSLEKDYIEIEYAEGEKLFIPIEQTNLIQKYIGSDPKNIKLDKISSKTWIKNKANAKKRIEEIADKLIELYSKRESIKGIKYPEDNELQLLFESEFPYDETPDQIRAIKEIKEDMMSFKVMDRLLCGDVGFGKTEVA

SEQ ID NO: 59 - DNA, 3378 bp, Borrelia burgdorferi
atgaatatagatgaagaactaacaacaatattaaagaataattctaatttaaaaaaaatgaaagaatttttagaacaaaatatatttttttcattaacaggatatgaaggatttttcaaggcttttttaattaaaaaaatcaaagaatatagcaaaaccgggaaaataatattaatagttaaagacgagcacacattagataaaatcaaaaacgatttacaagtaattacaaatcaaatctttgagcttaactattttagcccccttgtatacaagggcattggctcaaaaagtacgatctttaacgaaagaatcaaattcttattcaatttttataaaaaaaatcctggaatatatattacagtcttgaaatcattgcttagcaaaatacccgataaaaatacattactaaagaatatatataaaattgaaaaaaataccaatattaatacagcagacattgaaaaaactcttataacattgggatatgaaaaaacattaagagtaacaattccaggagaatttacagtaaaaggagaaattatagatatatacccttttggagaacaaaatccaataagaattgcactaaactttgacaaaatagaagaaataaaaaaatttaatcccttaacccaattaaaacacgataatgaaattttagaattccaaattcttccaaaaaaagaaattatttgggacgataaaactattaacaccttaaaaacaaaaattaaatctgttgaatataaaaagattcttgaagagttggattttaaaaaagaaacaaaaacagaagaaatgttttatccactagtagcaaatacttacttaggtgatgagattgaaaaacacacacctattgtaaactttgaaattaacaatttcgaaaaagaaattgaaaaaatacaccaagaatatgaaaagctttacaaagaagcagaagaagccggtaaaaatataattgatccaaaaagaattctcttaaattataaaaccttcaatctaaaaagcgatgttttattttcaaaaattaaaagccttaaatccaaagaaactatagagtttaaaatcgaaagtgagagaaactttttttcaaatatagcacttacaaaagaagaatttgaaaattggctgaaaaatggatttaaaatcattattgcagcagaatctgaatcacaaaaagaaaaacttaaatatattttcaaagaattgccaaaagtatcaattgaggttttaaaaatatctagctctttaataatagaaaaagaaaaaattgccattattcttgaatcaaacattttcaatacggggcaaaaaataaacaaagcctttgaatcttcaaaaacaaaagctattgactcttttgttgagattgagaaaaatagtcacgtagttcacataaaccatggaattggtatatttaggcaaataaagagaataaaaacaagctctcttgaaaaggattatattgaaattgaatacgctgaaggagaaaaactatttattccaattgaacaaacaaatttaatccaaaaatacattggaagtgatcctaaaaatatcaaattagataaaataagttctaaaacatggataaaaaacaaagcaaacgcaaaaaaaagaatcgaggagattgcagacaaattaatagaactttattcaaaaagagaaagcattaagggtattaaatacccagaagataatgaattacaattgttgtttgaatctgaatttccatacgatgaaactccagatcaaataagagcaataaaagaaataaaagaagatatgatgagctttaaagtaatggatcgccttctttgtggagatgttggatttggaaaaactgaagttgccatgagagctgcttttaaagccgtaatgggaaacaaacaggttattgtactctcaccaacaactatcttagcagaacagcatttcaatacatttaaaaaaagatttaaaaattttccaatcaaaatcgaagtattaagcagatttataaaaaataacgcagaaagccggatcttaaaagaacttaaaagtggaaaaattgatataatcataggaacgcacaaaattctttcaaaaaaattcacctgcaaaaatttagggttaataataattgatgaagaacaaagatttggtgtaaaagaaaaagaaaaacttaaagaaataagaatttcggttgattgccttgctctttctgcaacaccaattcccaggtctcttcacatgtcactaattaagcttagagatatttccgttttaaaaattccgcctcaaaacagagtaaaaatagaagcttatttagaatcatttagcgaacttttaataaaacatgcaattgagagtgaactgtctcgagatggtcaagtttttttagtaaatcataatattgaagaactgtattatttaaaaacactaattgaaagattaaccccttatgcaagaattgcaataattcatggaaaactaacaggagaagagattgaaaatataatgcacaattttattaaaaaagcgtatcaaattttattggcaacaacaataattgaaaatggaatagatattccaaatgcaaatacaataataataaataatgcaaacaagtttggacttgcacagctatatcaactaaaaggaagagttggaagaggatctcagaaagcttatgcttattttttgtaccaagacagcgaaaagctaaatgaacgctctattgaaagattaagagcaataacagaattttcagagctaggagcaggatttaaaatagcaatgaaagatatggaaataagaggtgttggcaatttacttggtagagaacaacatggggagattgagtcgattggactagattactatctaacaatgctaaataaagcaattgaaaagaaaatgggaaaaatctcatcagatgaagaagaggttgatattaaaattaactatagtggatttattcctgaaaattatgcaaaaaatgagcaggataaaatactaatctacaaaaaaatctttaaaattcaaactgaagaggaaagtaaaaaaataagatcagagctccacaacgactttggcccaatacccgaagaaataaacagtctattaatgttagctgaacttaaaattctagcaaaagatttaaacataacaaaattaaaagaaaaaaacagggctttggaaatagaatacaaaaatatagaaagcattcctatggaaaaaataatagaaatacttcaaaaacatcctaatttattaatattaaatccctcatatcaaaaatcaatatttttaagctttaaaaatattgaaaaatctgaaaaaataaattacatatataaaaatattaacttactaaaaacaagcacataa

SEQ ID NO: 60 - protein, 1125 aa, Borrelia burgdorferi
MNIDEELTTILKNNSNLKKMKEFLEQNIFFSLTGYEGFFKAFLIKKIKEYSKTGKIILIVKDEHTLDKIKNDLQVITNQIFELNYFSPLVYKGIGSKSTIFNERIKFLFNFYKKNPGIYITVLKSLLSKIPDKNTLLKNIYKIEKNTNINTADIEKTLITLGYEKTLRVTIPGEFTVKGEIIDIYPFGEQNPIRIALNFDKIEEIKKFNPLTQLKHDNEILEFQILPKKEIIWDDKTINTLKTKIKSVEYKKILEELDFKKETKTEEMFYPLVANTYLGDEIEKHTPIVNFEINNFEKEIEKIHQEYEKLYKEAEEAGKNIIDPKRILLNYKTFNLKSDVLFSKIKSLKSKETIEFKIESERNFFSNIALTKEEFENWLKNGFKIIIAAESESQKEKLKYIFKELPKVSIEVLKISSSLIIEKEKIAIILESNIFNTGQKINKAFESSKTKAIDSFVEIEKNSHVVHINHGIGIFRQIKRIKTSSLEKDYIEIEYAEGEKLFIPIEQTNLIQKYIGSDPKNIKLDKISSKTWIKNKANAKKRIEEIADKLIELYSKRESIKGIKYPEDNELQLLFESEFPYDETPDQIRAIKEIKEDMMSFKVMDRLLCGDVGFGKTEVAMRAAFKAVMGNKQVIVLSPTTILAEQHFNTFKKRFKNFPIKIEVLSRFIKNNAESRILKELKSGKIDIIIGTHKILSKKFTCKNLGLIIIDEEQRFGVKEKEKLKEIRISVDCLALSATPIPRSLHMSLIKLRDISVLKIPPQNRVKIEAYLESFSELLIKHAIESELSRDGQVFLVNHNIEELYYLKTLIERLTPYARIAIIHGKLTGEEIENIMHNFIKKAYQILLATTIIENGIDIPNANTIIINNANKFGLAQLYQLKGRVGRGSQKAYAYFLYQDSEKLNERSIERLRAITEFSELGAGFKIAMKDMEIRGVGNLLGREQHGEIESIGLDYYLTMLNKAIEKKMGKISSDEEEVDIKINYSGFIPENYAKNEQDKILIYKKIFKIQTEEESKKIRSELHNDFGPIPEEINSLLMLAELKILAKDLNITKLKEKNRALEIEYKNIESIPMEKIIEILQKHPNLLILNPSYQKSIFLSFKNIEKSEKINYIYKNINLLKTST

SEQ ID NO: 61 - DNA, 663 bp, Borrelia burgdorferi
ctggctgctacaccacttgttgatgatactgtaattggaaagttgagaactgcaaaaatcaacttttattcacttcttaatgaaactgggcttgatggtgtacccgcatttaaagaaggtgttgacctagctggaggtgcaatagacgaacaatttacataccactatataaaaaatgaagcgattattgagcttattagaatttggaacaaaaacaatagacaaaatagcaaattatctgcactacaacttagtggagctagagacaatgcatatacttcagcaattgaatgtttactgaaaaggtttgtggatagaggactgataatagagtataaaaatttaaggcttactctttctcctacgccacaacttaaattagaacttagcgtgaatattacttataactttagcattaatgctgttgctttagtaattactactcaagatatagttgattatcaaaacagcttaagtgcttaaaaggggggctaaaaatgcaattttatgatttaagagaagtttatttttcaattggtggtacgcagttacatagtggcaagctagagcttacaagcgaacctacaacaagagcagtgattagtagtgaagataaaggtatgcctgtaataagcttaagagatcccaaaacaataacttatg

SEQ ID NO: 62 - protein, 160 aa, Borrelia burgdorferi
LAATPLVDDTVIGKLRTAKINFYSLLNETGLDGVPAFKEGVDLAGGAIDEQFTYHYIKNEAIIELIRIWNKNNRQNSKLSALQLSGARDNAYTSAIECLLKRFVDRGLIIEYKNLRLTLSPTPQLKLELSVNITYNFSINAVALVITTQDIVDYQNSLSA

SEQ ID NO: 63 - DNA, 1113 bp, Borrelia burgdorferi
ttgccgcaagatacaattagtgtaagtttgcttgactctagaattcaagctagtaggcccaattattataatccacttttggtttacaaaacagccaaaattaaagttaataaagatgctgctaactataaaatattgaatttaaccgttaataactatgaaaaacaaattgaaactttagaaaaagataatgggaatggacaagatcagtttggaaaagaaaaaacactgcttaaaaccgcaatgtcgaattttttcaattcaagtgaagaatcattaaaatcagccgatctttttatttataaggataaacccgaagagttaaaaaaatatcttaaagtacatagacacacttttgttgtacttattaatactgagggtgataattccgatgatggacttaagatttataaagatgattatgataagtttaaaacaccttcaattttttttgtattctcaactaaagaacaagaaataaaagaactatttaaagataaaggcaatactgaaaaagaaagaaatattgctgtttacagcaataataaagacaatttacacctcaaatttataagtcaatatttacatcaagctagtatttttcatgctgtaaatccttatggcatgccgctggctgctacaccacttgttgatgatactgtaattggaaagttgagaactgcaaaaatcaacttttattcacttcttaatgaaactgggcttgatggtgtacccgcatttaaagaaggtgttgacctagctggaggtgcaatagacgaacaatttacataccactatataaaaaatgaagcgattattgagcttattagaatttggaacaaaaacaatagacaaaatagcaaattatctgcactacaacttagtggagctagagacaatgcatatacttcagcaattgaatgtttactgaaaaggtttgtggatagaggactgataatagagtataaaaatttaaggcttactctttctcctacgccacaacttaaattagaacttagcgtgaatattacttataactttagcattaatgctgttgctttagtaattactactcaagatatagttgattatcaaaacagcttaagtgcttaa

SEQ ID NO: 64 - protein, 370 aa, Borrelia burgdorferi
MPQDTISVSLLDSRIQASRPNYYNPLLVYKTAKIKVNKDAASYKILNLTVNNYEKQIETLEKENGNGEDQFGKEKTLLKTAMSNFFNSSEESLKSADLFIYKDKPEELKNYLKVHRHTFVVLINTEGDASDDGLKIYKDDYNKFKKPSTFFVFSTKEQEIKELFKDKGNTEKERNIAVYSNNKDNLHLKFISQYLHQASIFHAVNPYGMPLAATPLVDDTVIGKLRTAKINFYSLLNETGLDGVPAFKEGVDLAGGAIDEQFTYHYIKNEAIIELIRIWNKNNRQNSKLSALQLSGARDNAYTSAIECLLKRFVDRGLIIEYKNLRLTLSPTPQLKLELSVNITYNFSINAVALVITTQDIVDYQNSLSA

SEQ ID NO: 65 - DNA, 473 bp, Borrelia burgdorferi
caatatgaaaatactcttattaacttttttcaatcaagaagaagtaggacaataaattctcttgaatctgacttatacgcaactattgcctgtagaaaggctgtcaaaagaaatgacatgctaagcgctgaatttagtaaatttttaataaatgaattttttaaactagaaatcaaacattgtcctcatggacgaaaaatttattacaaaatatctaaatttgaacttgaaaaaaaagttgacagagcataaaataaaaccagagttcccatgatgaaaaaaatcaaatcagaaatcaacttgttaaagatagaaaaagacaaaaatttaattgagcttggaaaaatattaaaaaataataatatagtagaattaaaaaatttaaaccactatcccaatttaaaattagtagaaaaagaattgtatcaaatgaaaagcaatttaagcaaatcagaagaaaatgaaaacatatt

SEQ ID NO: 66 - protein, 83 aa, Borrelia burgdorferi
QYENTLINFFQSRRSRTINSLESDLYATIACRKAVKRNDMLSAEFSKFLINEFFKLEIKHCPHGRKIYYKISKFELEKKVDRA

SEQ ID NO: 67 - DNA, 1833 bp, Borrelia burgdorferi
atgaacaaaataagattcttagataaatacttggttcaaaaaatagcagcaggagaatcaatagacaggccatgttcaatattaagggaactactagacaattcaatagattctggagctactaaaattgaggtttttcttgaagaagggggaattcaaaaaatcttaataatagataatggaagcggaataagtaaagaagatttaaaaatctgctatctaccacacactacttcaaaaatatcatcggaagaagatttaagaaaaatagaaactctaggctttaggggagaggctctctctagtattgcaatttgctccaacatttcaataacaagctcaacaactagcaatgaaagctatcaaatagaagtagaaaatggaattgaaaaatgctttaaaaaacaacccgccataaacggaacaatagtagatgtcacaaaaatatttcacaacttcccagcaaggaaaagattcttaaagcaagaacccattgaaacaaaaatgtgtctaaaagttttagaagaaaaaataataacccaccccgaaatcaatttcgaaattaatttaaatcaaaagctaagaaaaatttactttaaagaatcgttaattgacagggttcaaaatgtatatggaaatgtaatagaaaataataaatttagggtcttaaaaaaagaacatgacaatataaaaatagaaatatttttagcaccagataacttttctaaaaaaagtaaaagacatattaaaacatttgtcaacagaagacctatcgatcaaaaagatctcttagaagcaataactaatggacacagcagaatactttctcctggcaacttcccaatatgttatttatttttagaaataaaccctgaatatattgactttaatgtacaccctcaaaaaaaagaagtaagattttacaatcttccatttttatttaaactaatatctgacaatattaataatttttttgataaaaatataaataactaccaagacataataataaaaagacaattaacagaagatgatcatttaatagaaatgacaaaccaaccagaaaactttaataaaatcaacacatatgatataccacaaaacaataatttagaaacagaagatgtaaacgagccaaacaaaaacacaacacaaagcaatattgaccttagaaggtataattcaattatacaaaatagaccaacactcagggaaaacattggaaacattttctctgacaattttttagaatttgaagaacctccaaataaaaatgaaaaagaagaaataaaatttaactatattggacaaatattctctgaatttttaatcgttgaaaaaataaatgaaatttacttcatagaccaacacgcagttcacgaaaaaataatatatgaaaaacttagaaattcaaaaaaaaatgttcaaaaacttctagtaccaattgaattcacagtagttgataaaaacatagaagaaattatagatagtgagattgaagaatacaaacaaatggacattataatctctaaaataggccctaaaaaatatcaacttgaatctattcctaatatttgtagtcaatatgaaaatactcttattaacttttttcaatcaagaagaagtaggacaataaattctcttgaatctgacttatacgcaactattgcctgtagaaaggctgtcaaaagaaatgacatgctaagcgctgaatttagtaaatttttaataaatgaattttttaaactagaaatcaaacattgtcctcatggacgaaaaatttattacaaaatatctaaatttgaacttgaaaaaaaagttgacagagcataa

SEQ ID NO: 68 - protein, 610 aa, Borrelia burgdorferi
MNKIRFLDKYLVQKIAAGESIDRPCSILRELLDNSIDSGATKIEVFLEEGGIQKILIIDNGSGISKEDLKICYLPHTTSKISSEEDLRKIETLGFRGEALSSIAICSNISITSSTTSNESYQIEVENGIEKCFKKQPAINGTIVDVTKIFHNFPARKRFLKQEPIETKMCLKVLEEKIITHPEINFEINLNQKLRKIYFKESLIDRVQNVYGNVIENNKFRVLKKEHDNIKIEIFLAPDNFSKKSKRHIKTFVNRRPIDQKDLLEAITNGHSRILSPGNFPICYLFLEINPEYIDFNVHPQKKEVRFYNLPFLFKLISDNINNFFDKNINNYQDIIIKRQLTEDDHLIEMTNQPENFNKINTYDIPQNNNLETEDVNEPNKNTTQSNIDLRRYNSIIQNRPTLRENIGNIFSDNFLEFEEPPNKNEKEEIKFNYIGQIFSEFLIVEKINEIYFIDQHAVHEKIIYEKLRNSKKNVQKLLVPIEFTVVDKNIEEIIDSEIEEYKQMDIIISKIGPKKYQLESIPNICSQYENTLINFFQSRRSRTINSLESDLYATIACRKAVKRNDMLSAEFSKFLINEFFKLEIKHCPHGRKIYYKISKFELEKKVDRA

SEQ ID NO: 69 - DNA, 984 bp, Borrelia burgdorferi
caattttacaacgaaattcttaaaaaagataataaaaattcttgcgatctaccaacaatgagtaaatatcttgatatattagaaaacacaaaaaccataataaagctatcttttaaaaatcatcccaaatatataatttattataaaattaattacccccttaaagtgttttgttcaacaatacaagactactatcaaacaatagcaaacaaactaaaactacggctataactaaactatcctactactatttaatcgtaaaaaaatatttctttgcaaattaatcaatttagaaatataaatgtaaagacatatctttttatttgataaataataaaaattactggggcactatttggaaaaatttttaaaagaaatattaagtatgaatagcaaaaataggctatcttcacacttaataattcttatttacacgctaaacaacattgacctaaattcaaaaaatattgggtacaaagagtttgaagagttaaataaagcacatgaagctgctttaagtagtagagaatcttagctagtgtagcttcaaagaagacatgacacttacttataaataaggaagcttttggattttaacaaaaatagtctggcttttttgcacatataaaacaactccattatttctaagataaatattttaagctccctggtaaagtaattcatttatcctagatttactcttccacttctatacgtcccgtcctgcttaatcattaatttttaaaattaaatgtttctttctagttacgcactatattgttactataacaaaaattgaatcttaaaaattaacatattactttaaaaaagtatacttataggagatgcttataaagcttaacaaacttatttttaccaatatatatatatctaatatctcttatacttagttgctcaatatctaaagatttaagtgataagctctcttcactaaaatctaatgatttttttaattcagacact

SEQ ID NO: 70 - protein, 76 aa, Borrelia burgdorferi
QFYNEILKKDNKNSCDLPTMSKYLDILENTKTIIKLSFKNHPKYIIYYKINYPLKVFCSTIQDYYQTIANKLKLRL

SEQ ID NO: 71 - DNA, 300 bp, Borrelia burgdorferi
atgcctgctttatgtggcaatacattattaaatctagaattgaataattattctcaaaaaaaactattaaaattttacaacgaaattcttaaaaaagataataaaaattcttgcgatctaccaacaatgagtaaatatcttgatatattagaaaacacaaaaaccataataaagctatcttttaaaaatcatcccaaatatataatttattataaaattaattacccccttaaagtgttttgttcaacaatacaagactactatcaaacaatagcaaacaaactaaaactacggctataa

SEQ ID NO: 72 - protein, 99 aa, Borrelia burgdorferi
MPALCGNTLLNLELNNYSQKKLLKFYNEILKKDNKNSCDLPTMSKYLDILENTKTIIKLSFKNHPKYIIYYKINYPLKVFCSTIQDYYQTIANKLKLRL

SEQ ID NO: 73 - DNA, 965 bp, Borrelia burgdorferi
cttctacacaataaagcccataagtatactttagaaagtcaatcccaacccgaatatattagtaaagttaacttttacatttatccaatatctaatatgcaaacagttgggcttattaatttaggcacaaaatatagcaaccatgcatacagtgcatctatagcatttaatgctagtgtaaaagcaattgaaattttaaaggaggaatacaaaattgccgcaagatacaattagtgtaagtttgcttgactctagaattcaagctagtaggcccaattattataatccacttttggtttacaaaacagccaaaattaaagttaataaagatgctgctaactataaaatattgaatttaaccgttaataactatgaaaaacaaattgaaactttagaaaaagataatgggaatggacaagatcagtttggaaaagaaaaaacactgcttaaaaccgcaatgtcgaattttttcaattcaagtgaagaatcattaaaatcagccgatctttttatttataaggataaacccgaagagttaaaaaaatatcttaaagtacatagacacacttttgttgtacttattaatactgagggtgataattccgatgatggacttaagatttataaagatgattatgataagtttaaaacaccttcaattttttttgtattctcaactaaagaacaagaaataaaagaactatttaaagataaaggcaatactgaaaaagaaagaaatattgctgtttacagcaataataaagacaatttacacctcaaatttataagtcaatatttacatcaagctagtatttttcatgctgtaaatccttatggcatgccgctggctgctacaccacttgttgatgatactgtaattggaaagttgagaactgcaaaaatcaacttttattcacttcttaatgaaactgggcttgatggtgtacccgcatttaaagaaggtg

SEQ ID NO: 74 - protein, 77 aa, Borrelia burgdorferi
LLHNKAHKYTLESQSQPEYISKVNFYIYPISNMQTVGLINLGTKYSNHAYSASIAFNASVKAIEILKEEYKIAARYN

SEQ ID NO: 75 - DNA, 570 bp, Borrelia burgdorferi
atgatttttactttagatatggttttaaaccatttaactcaaatattcaaagggtttaaggcgtatgcaactgaaaataattttgagtgcgatatcataaatacttacaatcacccgtacctttcaaaaatcacagctgctagctcaaatataatagcattgaaatttgatggtacagaaaatctatttgatcataattctagagccggtgcattttatgaaaatgctttggaattcagtttaaattttcaaatatatattattgctatagtgttaaacgctcaagattttgacgctaattcacgtatgttaatgctttatggtatgcttagcaattttctacacaataaagcccataagtatactttagaaagtcaatcccaacccgaatatattagtaaagttaacttttacatttatccaatatctaatatgcaaacagttgggcttattaatttaggcacaaaatatagcaaccatgcatacagtgcatctatagcatttaatgctagtgtaaaagcaattgaaattttaaaggaggaatacaaaattgccgcaagatacaattag

SEQ ID NO: 76 - protein, 189 aa, Borrelia burgdorferi
MIFTLDMVLNHLTQIFKGFKAYATENNFECDIINTYNHPYLSKITAASSNIIALKFDGTENLFDHNSRAGAFYENALEFSLNFQIYIIAIVLNAQDFDANSRMLMLYGMLSNFLHNKAHKYTLESQSQPEYISKVNFYIYPISNMQTVGLINLGTKYSNHAYSASIAFNASVKAIEILKEEYKIAARYN

SEQ ID NO: 77 - DNA, 403 bp, Borrelia burgdorferi
catgatttttataaaagtaatgctaaatttacaggatttatgttagataaattatttagtgatggaaaagaaataattaaaaaacttatgaaagaatataaagaattaaaaggataatatggaaattttatgaatagtttgacttacagaacatacaatatagaaagtataaaaaatgaatttttaaagataggatttagtgaagaagcaatagattttgtttttctgcataatgataattacaactttgaatttttaaaagaaaaattgattaatgtagaaaagaatttacaaaaggatatatctaatttagacattaagattgatactgtagaaaagaatttaaacactaaaatagacaatgttgaaaagaatttaaatctaaaaatagataatttagact

SEQ ID NO: 78 - protein, 38 aa, Borrelia burgdorferi
HDFYKSNAKFTGFMLDKLFSDGKEIIKKLMKEYKELKG

SEQ ID NO: 79 - DNA, 543 bp, Borrelia burgdorferi
ttggatattaaggttaataaaagagatttaagtgataatgaaaattatattattactgatagtgtattaaatcattataattctttaaaagaaaagttaaaaattaattctaaaaaggaaatctattgtaagttagaaactttaaaaattttaaaagaaattaaagacaatcattattataggtttgatggttataaaagttttgaagctttttctaaagattatagacttgcaagagcacaggtctataattacttaaaaattgctaatgcaatagaagatgggattatacaagaagaatttctaataaaaaatggtatactggaaacgttaattgtattaagaaataaagagagcaaaacaataaaaaaatcaaaacaaaacccgataaagcccttaagatttcagcttaaaagacaagaaagctatgatttttataaaagtaatgctaaatttacaggatttatgttagataaattatttagtgatggaaaagaaataattaaaaaacttatgaaagaatataaagaattaaaaggataa

SEQ ID NO: 80 - protein, 186 aa, Borrelia burgdorferi
MEIELNERVISRATDPDGEKKLITKEEIFAHYNDLKNRLKTNIKKKIFYKVESIRILKEIKDNEYYKLDGYKSFDAFIKDYQLARTQVYIYLKLAKALQAGILNEDYIIENGIYNSLDKIESQETPIVKKSKQNPIKPLRFQLKKQESYDFYKSNAKFTGFMLDKLFSDGKEIIKKLMKEYKELKG

SEQ ID NO: 81 - DNA, 1168 bp, Borrelia burgdorferi
caattcataaacaaatgtaaaaaaatagacaaaatgattttattaacaaacggaaaagatgcatttacaaatatttcagaagcactgatggcagaaaaattagaagaaattttttcaggattccaaataatcgacaaaatattcaaattcaaagaaataataataataatatcaaataaagacaaactaaaaaaagaattcgaaaaattgagtatttttaaaaacagaaaaataaaaattaaatctttagaaaatgcatatccttatacaaatcacgaaatgataatgcactttttatacaataataaaaatacaaaagatgatataaatccccacaacaatattttattagcaaatattgaagatctatacaatgcaaatcttgtcataaaaaataacaacccttataaagaaaaatttgtagctataaatggaaataaaaaaataaaaagtagaatactgaaggtaaaaattggaacctctttcagccagctaataaatgagaaaattgacacaaaaaaatatgaaatttttttaaacaacccagccaataaaataaaaatcgatacattaaacataccgataacaagagatatttatagtctcaccatattaaagaaaaagtcaatatacgacaaaattaaagcattgtttatatcaagctttgcaccattgcaaatggaaaatattattttttcattcttaaagaaagaaaaaataaataatagcggcaaattaaaaatatttaattatacagacaaagaagtagaagaagaaatacataaggttcaaaaagaaattaaaagcaaaattctaaatgaaagtctaacaaatgaagcaatatacactgaaaacaatttaaaagacatatactttgctatcatactatcactatcacctagcctgatattttctttcctaaataacacaaaattcatgacagacaccttgctgttaatatttattagtgtggcaatctatatacctataatgctaaaaattaattacaaatgtttgtctttttttgtttacgccgccttaatgataagcattatattgcccttggatttagaaattacactaaaaataatttccttattatttacttttttggtattcttttactttttcagactatcagcatttttggcaaaccctatattaattt

SEQ ID NO: 82 - protein, 389 aa, Borrelia burgdorferi
QFINKCKKIDKMILLTNGKDAFTNISEALMAEKLEEIFSGFQIIDKIFKFKEIIIIISNKDKLKKEFEKLSIFKNRKIKIKSLENAYPYTNHEMIMHFLYNNKNTKDDINPHNNILLANIEDLYNANLVIKNNNPYKEKFVAINGNKKIKSRILKVKIGTSFSQLINEKIDTKKYEIFLNNPANKIKIDTLNIPITRDIYSLTILKKKSIYDKIKALFISSFAPLQMENIIFSFLKKEKINNSGKLKIFNYTDKEVEEEIHKVQKEIKSKILNESLTNEAIYTENNLKDIYFAIILSLSPSLIFSFLNNTKFMTDTLLLIFISVAIYIPIMLKINYKCLSFFVYAALMISIILPLDLEITLKIISLLFTFLVFFYFFRLSAFLANPILI

SEQ ID NO: 83 - DNA, 2322 bp, Borrelia burgdorferi; identical to SEQ ID NO: 55

SEQ ID NO: 84 - protein, 773 aa, Borrelia burgdorferi; identical to SEQ ID NO: 56

SEQ ID NO: 85 - DNA, 704 bp, Borrelia burgdorferi
caaggaaaaactatgaaatacattttatctattgatcaaggtactactagctcgagagcaatggtatttgataaaaatgcaaacataaaggggtttgctcaaaaagaatttactcaaatttatccacaaccaagttgggtggaacatgatcctacagaaatatggggctcacaacttggagttataacagaagctatggcaaatgcaagaattttgcccaatgaaattgacgctattggaataactaatcaaagagaaactacggttatatgggaaaaaaatacaggaaagcccatctacaatgcaatagtatggcaagacagaagaactgcaaaaatttgtgaccaattaaaaaaagaaggaaaagataaaattattttggaaaaaacaggcttggtgctagattcttattttagtggaacaaaaataatgtggatattggataatgtagaaggtgccagacaaagagccgaaaatggcgaattatgctttggaacaatagatacatggatattgtggaacctgactcaaaaaaaagaacatgcaaccgattactctaatgcttcaagaacattattattaaacattaaaacattagagtgggatgatgaacttttaagcatattaaatgttccaagggcaattttgcctgaacttaaagaaagttctacaatatacggcaaaacagacaaagcactatttgg

SEQ ID NO: 86 - protein, 234 aa, Borrelia burgdorferi
QGKTMKYILSIDQGTTSSRAMVFDKNANIKGFAQKEFTQIYPQPSWVEHDPTEIWGSQLGVITEAMANARILPNEIDAIGITNQRETTVIWEKNTGKPIYNAIVWQDRRTAKICDQLKKEGKDKIILEKTGLVLDSYFSGTKIMWILDNVEGARQRAENGELCFGTIDTWILWNLTQKKEHATDYSNASRTLLLNIKTLEWDDELLSILNVPRAILPELKESSTIYGKTDKALF

SEQ ID NO: 87 - DNA, 1506 bp, Borrelia burgdorferi
atgaaatacattttatctattgatcaaggtactactagctcgagagcaatggtatttgataaaaatgcaaacataaaggggtttgctcaaaaagaatttactcaaatttatccacaaccaagttgggtggaacatgatcctacagaaatatggggctcacaacttggagttataacagaagctatggcaaatgcaagaattttgcccaatgaaattgacgctattggaataactaatcaaagagaaactacggttatatgggaaaaaaatacaggaaagcccatctacaatgcaatagtatggcaagacagaagaactgcaaaaatttgtgaccaattaaaaaaagaaggaaaagataaaattattttggaaaaaacaggcttggtgctagattcttattttagtggaacaaaaataatgtggatattggataatgtagaaggtgccagacaaagagccgaaaatggcgaattatgctttggaacaatagatacatggatattgtggaacctgactcaaaaaaaagaacatgcaaccgattactctaatgcttcaagaacattattattaaacattaaaacattagagtgggatgatgaacttttaagcatattaaatgttccaagggcaattttgcctgaacttaaagaaagttctacaatatacggcaaaacagacaaagcactatttggagcagaaattcctattgcaggaattgctggggatcaatttgcagcaacatttggacaagcctgccttaaaaagggtatggccaaaaacacttatggaactggctgcttcttaacggttaatataggaaaagaaccaatcattagccacgacaagcttttaacttcaattgcatggggaagaaaaaaatctgtaacctacgttcttgaaggaagtgtttttattggaggagctgtaatccagtggttaagagacggtcttgaatttttcagaaaaagctcagatgcagaagcgttggcaagctctgtctcagataatggtggagtttattttgtgccagcatttgttgggcttggtgcacctcactgggattcttatgcaagaggaacaatcatcggaataacaagaggctcaacaaaagctcacattacaagagctgctcttgagagcatcgcatttcaaagcttcgatatactaaataccatgaaaaaatccattcccaactttgaaattcaagaattaagagtagacgggggagcaagtcaaaacaatctattaatgcaatttcaagctgatcttttagaatgtaaagttgtaagaccaaaaataacagaaacaaccgctcttggcgctgcttatcttgcaggacttgcaacgggttattggcaaagcgccgaagaaatcgtaagcctttggcaagtagacaagatatttgaaccttcaatgccaaaaaatcaaaaagaaaagcttcttgagaattggaacaaagcggttggaaaagcaaaatcctggatacagaattctcatagttcataa

SEQ ID NO: 88 - protein, 449 aa, Borrelia burgdorferi
MKYILSIDQGTTSSRAMVFDKNANIKGFAQKEFTQIYPQPSWVEHDPTEIWGSQLGVITEAMANARILPNEIDAIGITNQRETTVIWEKNTGKPIYNAIVWQDRRTAKICDQLKKEGKDKIILEKTGLVLDSYFSGTKIMWILDNVEGARQRAENGELCFGTIDTWILWNLTQKKEHATDYSNASRTLLLNIKTLEWDDELLSILNVPRAILPELKESSTIYGKTDKALFGAEIPIAGIAGDQFAATFGQACLKKGMAKNTYGTGCFLTVNIGKEPIISHDKLLTSIAWGRKKSVTYVLEGSVFIGGAVIQWLRDGLEFFRKSSDAEALASSVSDNGGVYFVPAFVGLGAPHWDSYARGTIIGITRGSTKAHITRAALESIAFQSFDILNTMKKSIPNFEIQELRVDGGASQNNLLMQFQADLLECKVVRPKITETTALGAAYLAGLAT

SEQ ID NO: 89 - DNA, 311 bp, Borrelia burgdorferi
cagattaaagttttgcaaaaaaataataaatctaaaatattagccgagcagcttttcccaggaatatcttttttaaatttggataattataatgaaaaaatgaatgaaaagattgtatctaaaattatgagcactacggggtatatttattgcgaacttttagtttatttgagaacatatactatttattttgttaaaaaagatcttaatgatattattaatttacttattattaaaggacaatggaagcttatagagctttcaagagaaatgtctaacgacatgcatgctttgattaatatttatgcaag

SEQ ID NO: 90 - protein, 103 aa, Borrelia burgdorferi
QIKVLQKNNKSKILAEQLFPGISFLNLDNYNEKMNEKIVSKIMSTTGYIYCELLVYLRTYTIYFVKKDLNDIINLLIIKGQWKLIELSREMSNDMHALINIYA

SEQ ID NO: 91 - DNA, 1569 bp, Borrelia burgdorferi
atgatgaatttgggtttatttgattttatactttctatgtttagtattaataaagaacttacttctgagcaaataaagcaaaaaaggttaaaagaagttaaagttagtttaggcagagtaagtaatttttttaatgcttcaaaaattcaggctttacctcaattttctagatttctttataatttttataaaattttttctcccttaaggccatttgcacaaagatataaaaattctaataaaattgttcattttgttgttgaaaaatacttaaatgaaaaccaaaagaagtctttagattatatttattctttttctgcaagcgataacataaattttgcctcagatcttcctaaaaacttacataataatttatcttatttgtttaaaaacataactcaagaacaaattaaattgatagatgaaacttatgaagctttgcataatttttttgatttagtcttatatcaatatcatttggttcttaaaaattttgacaacttgcttccagaagatgattttgtgtataggcctagattcagctctataggttgtggagttattatagatgatcttaaagatttgttagaatgtatttcttgcattaagaatatttctatttggaaaaacctttatgacattattttagaaatttatgggaataaagaagattttcctattaagtctaatgtatggattaaggttatttcttctattttggatataaataagagtaaagaaattttatatctaataagatatgttagtggggatccagattatttccctatttctgttgggcaaaaacccaatccaatagcaagaatgttttttaatgatcttactaagcatgttgccactgaaattgaaaagattaaagttttgcaaaaaaataataaatctaaaatattagccgagcagcttttcccaggaatatcttttttaaatttggataattataatgaaaaaatgaatgaaaagattgtatctaaaattatgagcactacggggtatatttattgcgaacttttagtttatttgagaacatatactatttattttgttaaaaaagatcttaatgatattattaatttacttattattaaaggacaatggaagcttatagagctttcaagagaaatgtctaacgacatgcatgctttgattaatatttatgcaagtcttattgattttgattctaatttgggggaacaaggcggttatggcaatagaataaatgcattattgcacagagcttctttgggggataaatcttcggagaaattgttgttaaatataatagcagatgttaataaaaaggcgtttgctatatcaagcgaatattattccaaaatatattctattgagcagcgtttgcaagattgtctttcagactattcaaaagtctctttggaaagagagctgatttataattggaaagagcttgatatggatcttgctaaaagctatggaaacaatttaaactttggaggtatgatgaaaaatattttgggtagtttagctttatttttaaagttaatggatttatatttggagaaaaaatcttaa

SEQ ID NO: 92 - protein, 522 aa, Borrelia burgdorferi
MMNLGLFDFILSMFSINKELTSEQIKQKRLKEVKVSLGRVSNFFNASKIQALPQFSRFLYNFYKIFSPLRPFAQRYKNSNKIVHFVVEKYLNENQKKSLDYIYSFSASDNINFASDLPKNLHNNLSYLFKNITQEQIKLIDETYEALHNFFDLVLYQYHLVLKNFDNLLPEDDFVYRPRFSSIGCGVIIDDLKDLLECISCIKNISIWKNLYDIILEIYGNKEDFPIKSNVWIKVISSILDINKSKEILYLIRYVSGDPDYFPISVGQKPNPIARMFFNDLTKHVATEIEKIKVLQKNNKSKILAEQLFPGISFLNLDNYNEKMNEKIVSKIMSTTGYIYCELLVYLRTYTIYFVKKDLNDIINLLIIKGQWKLIELSREMSNDMHALINIYASLIDFDSNLGEQGGYGNRINALLHRASLGDKSSEKLLLNIIADVNKKAFAISSEYYSKIYSIEQRLQDCLSDYSKVSLERELIYNWKELDMDLAKSYGNNLNFGGMMKNILGSLALFLKLMDLYLEKKS

SEQ ID NO: 93 - DNA, 699 bp, Borrelia burgdorferi
catccttttggttctagcgatgctaaaaaagtgattgaagcttttgcttctctttataataatggaacttggagtgatatgattgccgagattactattaagtcaaagcaatatccaaaaaatgaaaaagtttacagaattacgcttgattctcagctttttaatgttgctatgaaaaaaataattgaaaaatatcctaaaataaaaagtgcaagttttgcatttaattcgttaattaactaaaaaaatattttaaaagctagcagtttaaaaccactagctttttaaattaattataaatttcagacttggatttgctatatagtgtctttaagtttattaagtgtggttttggaatctgcttgattaaaaattccatcaaatccgctagcaagtatgcttttgtaagtatttagctcttctggatctttttcttttttatctttattttctttaattttcgcaagcagatcttttatttcatcttcttttagctctgatagaaatttttgtacagattcttctattttgctttcgtctccaccgctatttttaaaatgtaagccaatatttggatctactagtttttcttttaataagtttgttaaaaatgttagtgtttctttttccgtgttgtttaagtttaatctgcttactaatttttctaaaggtgttggatctggtaccacgcgtatcgat

SEQ ID NO: 94 - protein, 80 aa, Borrelia burgdorferi
HPFGSSDAKKVIEAFASLYNNGTWSDMIAEITIKSKQYPKNEKVYRITLDSQLFNVAMKKIIEKYPKIKSASFAFNSLIN

SEQ ID NO: 95 - DNA, 849 bp, Borrelia burgdorferi
ttgaaaagagtcattgtatcctttgtggttttaatcctagggtgtaatttagatgataattcaaaaatggagagaaagggtagtaataagcttattagagaaagtggatcagataggcggggtcaagaaaatagagccttgggggcgatgaattttgggcttttttctggagattctggtgtagtttatgatttgcaaaattatgaaactttaaaagctcttgaaaataaaaataaatttattgattactctaaaatagagtttttagaaggaacaaaatcaataaatgcttttatttgggcagtttctgttcgttggataaaaattaaagccagagatttgtttggggagtgtggagattttattaaagagcttaagggcattaagtattcttatcttgtttctcctgttgatggaagctatatttcttatgccatgcctataatagtttttgaaactactagagagagtgatccgctttattctgtttctgggtttaaattaataagcaagggaaatgatataaattttaatgaaaataaaagcggattttggggaagacttccaatgtctgaaaaatcagttgaatctgggcttgtaaccgcatatccttttggttctagcgatgctaaaaaagtgattgaagcttttgcttctctttataataatggaacttggagtgatatgattgccgagattactattaagtcaaagcaatatccaaaaaatgaaaaagtttacagaattacgcttgattctcagctttttaatgttgctatgaaaaaaataattgaaaaatatcctaaaataaaaagtgcaagttttgcatttaattcgttaattaactaa

SEQ ID NO: 96 - protein, 282 aa, Borrelia burgdorferi
MKRVIVSFVVLILGCNLDDNSKMERKGSNKLIRESGSDRRGQENRALGAMNFGLFSGDSGVVYDLQNYETLKALENKNKFIDYSKIEFLEGTKSINAFIWAVSVRWIKIKARDLFGECGDFIKELKGIKYSYLVSPVDGSYISYAMPIIVFETTRESDPLYSVSGFKLISKGNDINFNENKSGFWGRLPMSEKSVESGLVTAYPFGSSDAKKVIEAFASLYNNGTWSDMIAEITIKSKQYPKNEKVYRITLDSQLFNVAMKKIIEKYPKIKSASFAFNSLIN

SEQ ID NO: 97 - DNA, 321 bp, Borrelia burgdorferi
cacactgccctttcagaatcaatttatttaagcaaattggtggacaaggtttatcttattgtaagaaaaaataatcttagagctattgctatgttaagagatagtgttgctaagttacctaatattgaaattttgtataattcagaagccatagaagtagatggtaaatcttctgtttcttcggttaagatttttaataaaaaagataatgttgtttatgaattagaagtgagtgctgtatttatggctgttggctataagccaaatacagaatttttaaagggatttttggatttggacgaaggcaatcgtttttaattg

SEQ ID NO: 98 - protein, 105 aa, Borrelia burgdorferi
HTALSESIYLSKLVDKVYLIVRKNNLRAIAMLRDSVAKLPNIEILYNSEAIEVDGKSSVSSVKIFNKKDNVVYELEVSAVFMAVGYKPNTEFLKGFLDLDEGNRF

SEQ ID NO: 99 - DNA, 981 bp, Borrelia burgdorferi
atgttggaatttgaaactattgatataaatctaaccaagaagaaaaatctatctcaaaaagaggtagattttattgaagatgtaataattgtaggatctggcccggctggactaacagctgggatttattctgttatgagtaattataaggctgctattttggaaggtcctgaacccgggggacagcttactacaaccacagaagtttacaattatcctggctttaaaaatggaataagtggtagaaatttgatgttaaatatgagggagcaagtagtaaatcttggggctaaaacttttcccgaaaccgttttttctataaaaaggaagggtaatattttttacctttatacagaaaattatatttataaaagtaaagctgttattattgctgtgggatcaaaacccaaaaaacttgaaactcttaaaaattcgggtttattttggaataaaggtatttctgtttgtgctatttgtgatggacatctttttaaagggaaaagggttgcagtaattggtggaggcaacactgccctttcagaatcaatttatttaagcaaattggtggacaaggtttatcttattgtaagaaaaaataatcttagagctattgctatgttaagagatagtgttgctaagttacctaatattgaaattttgtataattcagaagccatagaagtagatggtaaatcttctgtttcttcggttaagatttttaataaaaaagataatgttgtttatgaattagaagtgagtgctgtatttatggctgttggctataagccaaatacagaatttttaaagggatttttggatttggacgaagagggatttattgtcactaaagatgttgttaaaacaagcgttgatggtgttttttcatgtggagatgttagcaataaactttatgctcaagccattactgctgctgctgaggggtttattgcatctgttgagttaggaaattttttaaaatag

SEQ ID NO: 100 - protein, 326 aa, Borrelia burgdorferi
MLEFETIDINLTKKKNLSQKEVDFIEDVIIVGSGPAGLTAGIYSVMSNYKAAILEGPEPGGQLTTTTEVYNYPGFKNGISGRNLMLNMREQVVNLGAKTFPETVFSIKRKGNIFYLYTENYIYKSKAVIIAVGSKPKKLETLKNSGLFWNKGISVCAICDGHLFKGKRVAVIGGGNTALSESIYLSKLVDKVYLIVRKNNLRAIAMLRDSVAKLPNIEILYNSEAIEVDGKSSVSSVKIFNKKDNVVYELEVSAVFMAVGYKPNTEFLKGFLDLDEEGFIVTKDVVKTSVDGVFSCGDVSNKLYAQAITAAAEGFIASVELGNFLK

SEQ ID NO: 101 - DNA, 225 bp, Borrelia burgdorferi
catatgatattatatcaaaatcaattaaaatttttaaaattgttggtattttttttattaatatcttgcacttccttaaacgttgagcacgatcaatttggaaaaacatttagaatataccaaagcttaaataaaaatgcagaacttaagggtatttttaattataaaacaggaataactaaaatagtattatacacaaggtttagaaaccatagtataacagaa

SEQ ID NO: 102 - protein, 75 aa, Borrelia burgdorferi
HMILYQNQLKFLKLLVFFLLISCTSLNVEHDQFGKTFRIYQSLNKNAELKGIFNYKTGITKIVLYTRFRNHSITE

SEQ ID NO: 103 - DNA, 498 bp, Borrelia burgdorferi
atgatattatatcaaaatcaattaaaatttttaaaattgttggtattttttttattaatatcttgcacttccttaaacgttgagcacgatcaatttggaaaaacatttagaatataccaaagcttaaataaaaatgcagaacttaagggtatttttaattataaaacaggaataactaaaatagtattatacacaaggtttagaaaccatagtataacagaacagaatcctttattgctgctggatggaactaaaattgagggaaaagtaagctacaaaagagataataaccatttttttggcaactggattaattattcatcatttgttttgaccaaatctttattggaaaggatgattaaagaagaagatgcttcttataagaataaggaggttaaaattagaattggattagaagatttaagcttgaaaaaatataaaattttggactttctagtaatggttgaatcgattgaaaataaagattataaaagttaa

SEQ ID NO: 104 - protein, 165 aa, Borrelia burgdorferi
MILYQNQLKFLKLLVFFLLISCTSLNVEHDQFGKTFRIYQSLNKNAELKGIFNYKTGITKIVLYTRFRNHSITEQNPLLLLDGTKIEGKVSYKRDNNHFFGNWINYSSFVLTKSLLERMIKEEDASYKNKEVKIRIGLEDLSLKKYKILDFLVMVESIENKDYKS

SEQ ID NO: 105 - DNA, 896 bp, Borrelia burgdorferi
cttgatgtgcttagtgatggttatgggtttttgagaactgcatcaaattcttatctttctggaggcaatgatgtttacgtttctccctctcagattagactttttaatttaaggacaggtgatattttatatggccaaattagatcgcctagagatggtgagagattttttgcaatggttaaaattaaatctattaatgatcaagatcctacgtttgctcaaaacagaataccttttgataatttaactcctctttatcccaatgtgaaattgaatcttgaatatgaaaattgcaatatttctacaaggcttattaatcttttttcacctataggtaaggggcaaagggctttaatagtttctcctccaaaagcgggaaagactaccctgcttcaaaaaatagctaatgcaataactactaattattcagatgttattttaatgatactacttattgatgagaggccagaagaagttacagatatgattcgtagcgttaaaggcgaagtaattgcatctaattttgatgagcaggctagtaggcatgttcaggtagcagagatggttattgaaaaggctaaaaggcttgttgaaaacaaaaaagatgttgttattttgcttgattctattacaaggcttgcaagggcatataatcaaactatgccaacttctggtaaaattttatcgggtggggtagattctaatgctcttcataagccaaagaggttttttgggtctgctagaaatatagaagaagggggaagtttaactattatagctactgctttggttgatactggtagtaaaatggatgaagttatttttgaagaatttaaaagtaccggtaatatggaattaattcttgatagaagtttggcagacagaagactttttcc

SEQ ID NO: 106 - protein, 298 aa, Borrelia burgdorferi
LDVLSDGYGFLRTASNSYLSGGNDVYVSPSQIRLFNLRTGDILYGQIRSPRDGERFFAMVKIKSINDQDPTFAQNRIPFDNLTPLYPNVKLNLEYENCNISTRLINLFSPIGKGQRALIVSPPKAGKTTLLQKIANAITTNYSDVILMILLIDERPEEVTDMIRSVKGEVIASNFDEQASRHVQVAEMVIEKAKRLVENKKDVVILLDSITRLARAYNQTMPTSGKILSGGVDSNALHKPKRFFGSARNIEEGGSLTIIATALVDTGSKMDEVIFEEFKSTGNMELILDRSLADRRLF

SEQ ID NO: 107 - DNA, 1548 bp, Borrelia burgdorferi
atggataaaaaaaatggtggatttgatttagaaagtgaaataaggcggttggatgtttctaaagagtttaagattgaagataatttgaaaaaaaaagtggttaaagttgttgctaaaaaggattctgcatctagcgtgacaaaatctgcagatttaagtggcgtaaaggattcaaacggggtaatattttctgattttgattatgatattccttcttcaggtttagaaaataatattaaaactttagagcaaagtaatatcatcaaattttttaatggtaaagattatgtagagattgaaaaactttatgataagccaattacagaggttagaaaaattgttgaaggtcttgggaccaatcatactattgctgtaacaatgaaaaaaaccgaattaatatttttattggtgaaaatattaagtgagaataatattgatgttttatttacaggtgtgcttgatgtgcttagtgatggttatgggtttttgagaactgcatcaaattcttatctttctggaggcaatgatgtttacgtttctccctctcagattagactttttaatttaaggacaggtgatattttatatggccaaattagatcgcctagagatggtgagagattttttgcaatggttaaaattaaatctattaatgatcaagatcctacgtttgctcaaaacagaataccttttgataatttaactcctctttatcccaatgtgaaattgaatcttgaatatgaaaattgcaatatttctacaaggcttattaatcttttttcacctataggtaaggggcaaagggctttaatagtttctcctccaaaagcgggaaagactaccctgcttcaaaaaatagctaatgcaataactactaattattcagatgttattttaatgatactacttattgatgagaggccagaagaagttacagatatgattcgtagcgttaaaggcgaagtaattgcatctaattttgatgagcaggctagtaggcatgttcaggtagcagagatggttattgaaaaggctaaaaggcttgttgaaaacaaaaaagatgttgttattttgcttgattctattacaaggcttgcaagggcatataatcaaactatgccaacttctggtaaaattttatcgggtggggtagattctaatgctcttcataagccaaagaggttttttgggtctgctagaaatatagaagaagggggaagtttaactattatagctactgctttggttgatactggtagtaaaatggatgaagttatttttgaagaatttaaaagtaccggtaatatggaattaattcttgatagaagtttggcagacagaagactttttcctgctattaatattaaaaagtcaggtaccagaaaagaagaattgcttcttagtgaagaggagcgttctaagattttgcttattagaagaatacttggaggtgttgatgattatgagggagttgaagttctgatagaaaagatgaaaaaaagcaaaaacaatgaaattttcttgaagacaatgagcaatggtaattga

SEQ ID NO: 108 - protein, 515 aa, Borrelia burgdorferi
MDKKNGGFDLESEIRRLDVSKEFKIEDNLKKKVVKVVAKKDSASSVTKSADLSGVKDSNGVIFSDFDYDIPSSGLENNIKTLEQSNIIKFFNGKDYVEIEKLYDKPITEVRKIVEGLGTNHTIAVTMKKTELIFLLVKILSENNIDVLFTGVLDVLSDGYGFLRTASNSYLSGGNDVYVSPSQIRLFNLRTGDILYGQIRSPRDGERFFAMVKIKSINDQDPTFAQNRIPFDNLTPLYPNVKLNLEYENCNISTRLINLFSPIGKGQRALIVSPPKAGKTTLLQKIANAITTNYSDVILMILLIDERPEEVTDMIRSVKGEVIASNFDEQASRHVQVAEMVIEKAKRLVENKKDVVILLDSITRLARAYNQTMPTSGKILSGGVDSNALHKPKRFFGSARNIEEGGSLTIIATALVDTGSKMDEVIFEEFKSTGNMELILDRSLADRRLFPAINIKKSGTRKEELLLSEEERSKILLIRRILGGVDDYEGVEVLIEKMKKSKNNEIFLKTMSNGN

SEQ ID NO: 109 - DNA, 318 bp, Borrelia burgdorferi
ccgattgttaaggctgctggtgaggctgagcaggatggagagaagcctgaggatgctaaaaatccgattgctgctgctattgggaagggtaatggggatggtgcggagtttgatcaggatgagatgaagaaggatgatcagattgctgctgctattgctttgagggggatggctaaggatggaaagtttgctgtgaagggtaataatgagaaagagaaggctgagggggctattaaagaagttagcgagttgttggataagctggtaacagctgtaaagacagctgagggggcttcaagtggtactgatgcaattgga

SEQ ID NO: 110 - protein, 106 aa, Borrelia burgdorferi
PIVKAAGEAEQDGEKPEDAKNPIAAAIGKGNGDGAEFDQDEMKKDDQIAAAIALRGMAKDGKFAVKGNNEKEKAEGAIKEVSELLDKLVTAVKTAEGASSGTDAIG

SEQ ID NO: 111 - DNA, 507 bp, Borrelia burgdorferi
gagggggctattaagggagctgctgcaattggagaagttgtggataatgctggtgctgcgaaggctgctgataaggatagtgtgaaggggattgctaaggggataaaggagattgttgaagctgctggggggagtgaaaagctgaaagctgctgctgctgaaggggagaataataaaaaggcagggaagttgtttgggaaagttgatggtgctgctggggacagtgaggctgctagcaaggcggctggtgctgttagtgctgttagtggggagcagatattaagtgcgattgttaaggctgctggtgaggctgagcaggatggagagaagcctgaggatgctaaaaatccgattgctgctgctattgggaagggtaatggggatggtgcggagtttgatcaggatgagatgaagaaggatgatcagattgctgctgctattgctttgagggggatggctaaggatggaaagtttgctgtgaagggtaataatgagaaagagaaggct

SEQ ID NO: 112 - protein, 169 aa, Borrelia burgdorferi
EGAIKGAAAIGEVVDNAGAAKAADKDSVKGIAKGIKEIVEAAGGSEKLKAAAAEGENNKKAGKLFGKVDGAAGDSEAASKAAGAVSAVSGEQILSAIVKAAGEAEQDGEKPEDAKNPIAAAIGKGNGDGAEFDQDEMKKDDQIAAAIALRGMAKDGKFAVKGNNEKEKA

SEQ ID NO: 113 - DNA, 558 bp, Borrelia burgdorferi
gagggggctattaaagaagttagcgagttgttggataagctggtaacagctgtaaagacagctgagggggcttcaagtggtactgatgcaattggagaagttgtggataatgatgctaaggttgctgataaggcgagtgtgacggggattgctaaggggataaaggagattgttgaagctgctagggggagtgaaaagctgaaagttgctgctgctaaagagggcaatgaaaaggcagggaagttgtttgggaaggctggtgctaatgctcatggggacagtgaggctgctagcaaggcggctggtgctgttagtgctgttagtggggagcagatattaagtgcgattgttaaggctgcggatgcggctgagcaggatggaaagaagcctgcagatgctacaaatccgattgctgctgctattgggaataaagatgaggatgcggattttggtgatgggatgaagaaggatgatcagattgctgctgctattgctttgagggggatggctaaggatggaaagtttgctgtgaagggtaataatgagaaagggaaggct

SEQ ID NO: 114 - protein, 186 aa, Borrelia burgdorferi
EGAIKEVSELLDKLVTAVKTAEGASSGTDAIGEVVDNDAKVADKASVTGIAKGIKEIVEAARGSEKLKVAAAKEGNEKAGKLFGKAGANAHGDSEAASKAAGAVSAVSGEQILSAIVKAADAAEQDGKKPADATNPIAAAIGNKDEDADFGDGMKKDDQIAAAIALRGMAKDGKFAVKGNNEKGKA

SEQ ID NO: 115 - DNA, 8370 bp, Borrelia burgdorferi
atgacaaacccctccactgccaccttattaacaactttctttgtttttattaattgtaaaagccaagttgctgataaggcgagtgtgacggggattgctaagggaataaaggagattgttgaagctgctggggggagtgaaaagctgaaagttgctgctgctgaaggggagaataatgaaaaggcagggaagttgtttgggaaggctggtgctggtaatgctggggacagtgaggctgctagcaaggcggctggtgctgttagtgctgttagtggggagcagatattaagtgcgattgttaaggctgctggtgaggctgcgcaggatggaaagaagcctggggaggctaaaaatccgattgctgctgctattgggaagggtaatgaggatggtgcggagtttaaggatgagatgaagaaggatgatcagattgctgctgctattgctttgagggggatggctaaggatggaaagtttgctgtgaagaatgatgagaaagggaaggctgagggggctattaagggagctggcgagttgttggataagctggtaaaagctgtaaagacagctgagggggcttcaagtggtactgctgcaattggagaagttgtggctgatgataatgctgcgaaggttgctgataaggcgagtgtgaaggggattgctaaggggataaaggagattgttgaagctgctggggggagtaaaaagctgaaagttgctgctgctaaagagggcaatgaaaaggcagggaagttgtttgggaaagttgatgctgctcatgctggggacagtgaggctgctagcaaggcggctggtgctgttagtgctgttagtggggagcagatattaagtgcgattgttaaggctgctggtgcggctgctggtgatcaggagggaaagaagcctggggatgctaaaaatccgattgctgctgctattgggaagggtgatgcggagaatggtgcggagtttaatcatgatgggatgaagaaggatgatcagattgctgctgctattgctttgagggggatggctaaggatggaaagtttgctgtgaagagtggtggtggtgagaaagggaaggctgagggggctattaagggagctgctgagttgttggataagctggtaaaagctgtaaagacagctgagggggcttcaagtggtactgatgcaattggagaagttgtggctaatgctggtgctgcaaaggttgctgataaggcgagtgtgacggggattgctaaggggataaaggagattgttgaagctgctggggggagtgaaaagctgaaagttgctgctgctacaggggagagtaataaaggggcagggaagttgtttgggaaggctggtgctggtgctaatgctggggacagtgaggctgctagcaaggcggctggtgctgttagtgctgttagtggggagcagatattaagtgcgattgttaaggctgctgatgcggctgatcaggagggaaagaagcctggggatgctacaaatccgattgctgctgctattgggaagggtaatgaggagaatggtgcggagtttaaggatgagatgaagaaggatgatcagattgctgctgctattgctttgagggggatggctaaggatggaaagtttgctgtgaaggatggtggtgagaaagggaaggctgagggggctattaagggagctgctgagttgttggataagctggtaaaagctgtaaagacagctgagggggcttcaagtggtactgatgcaattggagaagttgtggataatgctgcgaaggctgctgataaggcgagtgtgacggggattgctaaggggataaaggagattgttgaagctgctggggggagtgaaaagctgaaagttgctgctgctacaggggagaataataaagaggcagggaagttgtttgggaaggctggtgctgatgctaatggggacagtgaggctgctagcaaggcggctggtgctgttagtgctgttagtggggagcagatattaagtgcgattgttaaggctgcggctgctggtgcggctgatcaggatggagagaagcctggggatgctaaaaatccgattgctgctgctattgggaagggtaatgcggatgatggtgcggattttggtgatgggatgaagaaggatgatcagattgctgctgctattgctttgagggggatggctaaggatggaaagtttgctgtgaagaaggatgagaaagggaaggctgagggggctattaagggagctagcgagttgttggataagctggtaaaagctgtaaagacagctgagggggcttcaagtggtactgctgcaattggagaagttgtggataatgctgcgaaggctgctgataaggatagtgtgacggggattgctaaggggataaaggagattgttgaagctgcaggggggagtgaaaagctgaaagttgctgctgctaaaggggagaataataaaggggcagggaagttgtttgggaaggctggtgctaatgctcatggggacagtgaggctgctagcaaggcggctggtgctgttagtgctgttagtggggaacagatattaagtgcgattgttaaggctgctggtgaggctgctggtgatcaggagggaaagaagcctgaggaggctaaaaatccgattgctgctgctattggggataaagatggggatgcggagtttaatcaggatgggatgaagaaggatgatcagattgctgctgctattgctttgagggggatggctaaggatggaaagtttgctgtgaaggatggtggtgagaaagagaaggctgagggggctattaaaggagttagcgagttgttggataagctggtaaaagctgtaaagacagctgagggggcttcaagtggtactgctgcaattggagaagttgtggctgatgctgctaaggttgctgataaggcgagtgtgacggggattgctaaggggataaaggagattgttgaagctgctggggacagtgaggctgctagcaaggcagctggtgctgttagtgctgttagtggggagcagatattaagtgcgattgttaaggctgcggctgctggtgcggctgagcaggatggagagaagcctgcagaggctaaaaatccgattgctgctgctattgggaagggtgatggggatgcggattttggtgaggatgggatgaagaaggatgatcagattgctgctgctattgctttgagggggatggctaaggatggaaagtttgctgtgaagaatgatgagaaagggaaggctgagggggctattaagggagctgctgcaattggagaagttgtggataatgctggtgctgcgaaggctgctgataaggatagtgtgaaggggattgctaaggggataaaggagattgttgaagctgctggggggagtgaaaagctgaaagctgctgctgctgaaggggagaataataaaaaggcagggaagttgtttgggaaagttgatggtgctgctggggacagtgaggctgctagcaaggcggctggtgctgttagtgctgttagtggggagcagatattaagtgcgattgttaaggctgctggtgaggctgagcaggatggagagaagcctgaggatgctaaaaatccgattgctgctgctattgggaagggtaatggggatggtgcggagtttgatcaggatgagatgaagaaggatgatcagattgctgctgctattgctttgagggggatggctaaggatggaaagtttgctgtgaagggtaataatgagaaagagaaggctgagggggctattaaagaagttagcgagttgttggataagctggtaacagctgtaaagacagctgagggggcttcaagtggtactgatgcaattggagaagttgtggataatgatgctaaggttgctgataaggcgagtgtgacggggattgctaaggggataaaggagattgttgaagctgctagggggagtgaaaagctgaaagttgctgctgctaaagagggcaatgaaaaggcagggaagttgtttgggaaggctggtgctaatgctcatggggacagtgaggctgctagcaaggcggctggtgctgttagtgctgttagtggggagcagatattaagtgcgattgttaaggctgcggatgcggctgagcaggatggaaagaagcctgcagatgctacaaatccgattgctgctgctattgggaataaagatgaggatgcggattttggtgatgggatgaagaaggatgatcagattgctgctgctattgctttgagggggatggctaaggatggaaagtttgctgtgaagggtaataatgagaaagggaaggctgagggggcttcaagtggtactgatgcaattggagaagttgtggataatgatgcgaaggctgctgataaggcgagtgtgacggggattgctaaggggataaaggagattgttgaagctgctggggggagtgaaaagctgaaagctgttgctgctgctacaagggagaataataaagaggcagggaagttgtttgggaaagttgatgatgctcatgctggggacagtgaggctgctagcaaggcggctggtgctgttagtgctgttagtggggagcagatattaagtgcgattgttacggctgcggctgctggtgagcaggatggagagaagcctgcagaggctacaaatccgattgctgctgctattgggaagggtaatgaggatggtgcggattttggtaaggatgagatgaagaaggatgatcagattgctgctgctattgctttgagggggatggctaaggatggaaagtttgctgtgaagagtaatgatggtgagaaagggaaggctgagggggctattaaggaagttagcgagttgttggataagctggtaaaagctgtaaagacagctgagggggcttcaagcggtactgatgcaattggagaagttgtggctaatgctggtgctgcgaaggctgctgataaggcgagtgtgacggggattgctaaggggataaaggagattgttgaagctgctggggggagtaaaaagctgaaagctgctgctgctgaaggggagaataataaaaaggcagggaagttgtttgggaaggctggtgctggtgctggtgctaatggggacagtgaggctgctagcaaggcggctggtgctgttagtgctggttagtgtggggagcagatattaagtgcgattgttacggctgctggtgcggctgctagtgaggctgatcaggagggaaagaagcctgcagatgctacaaatccgattgctgctgctattgggaagggtgatgcggagaatggtgcggattttggtgatgggatgaagaaggatgatcagattgctgctgctattgctttgagggggatggctaaggatggaaagtttgctgtgaagaatgatgatgagaaagggaaggctgagggggctattaagggagctagcgagttgttggataagctggtaacagctgtaaagacagctgagggggcttcaagtggtactgatgcaattggagaagttgtggctgatgctgcgaaggctgctgataaggatagtgtgaaggggattgctaaggggataaaggagattgttgaagctgctggggggagtgaaaagctgaaagttgctgctgctaaagagggcaatgaaaaggcagggaagttgtttgggaaggttggtgatgctgctcatgctggggacagtgaggctgctagcaaggcggctggtgctgttagtgctgttagtggggagcagatattaagtgcgattgttacggctgctggtgcggctgagcaggagggaaagaagcctgcagaggctaaaaatccgattgctgctgctattgggaagggtaatgagaatggtgcggagtttaaggatgagatgaagaaggatgatcagattgctgctgctattgctttgagggggatggctaaggatggaaagtttgctgtgaagaaggataataatgagaaagggaaggctgagggggctattaaggaagttagcgagttgttggataagctggtaacagctgtaaagacagctgaggaggcttcaagtggtactgctgcaattggagaagttgtggctgatgatgctgctgcgaaggctgctgataaggagagtgtgaaggggattgctaaggggataaaggagattgttgaagctgctggggggagtaaaaagctgaaagttgctgctgctacaggggagaataataaaaaggcagggaagttgtttgggaaagttgatgctggtaatgctggggacagtgaggctgctagcaaggcggctggtgctgttagtggggagcagatattaagtgcgattgttaaggctgctggtgcggctgctggtgatcaggagggaaagaagcctggggatgctaaaaatccgattgctgctgctattgggaagggtgatgcggagaatggtgcggagtttgatcatgagatgaagaaggatgatcagattgctgctgctattgctttgagggggatggctaaggatggaaagtttgctgtgaagagtggtgatgagaaagggaaggctgagggggctattaagggagctagcgagttgttggataagctggtaaaagctgtgtaaagacagctgagggggcttcaagtggtactgatgcaattggagaagttgtggctaatgatgctgctgcgaaggttgctgataaggagagtgtgacggggattgctaaggggataaaggagattgttgaagctgctggggggagtgaaaagctgaaagttgctgctgctacaagggagaataataaaaaggcagggaagttgtttgggaaagctggtgatgctgctaatggggacagtgaggctgctagcaaggcggctggtgctgttagtgctgttagtggggagcagatattaagtgcgattgttacggctgcagctgctggtgcggctgagcaggagggaaagaagcctgaggaggctaaaaatccgattgctgctgctattgggaagggtaatgcggatgatggtgcggagtttaataaggaggggatgaagaaggatgatcagattgctgctgctattgctttgagggggatggctaaggatggaaagtttgctgtgaagagtggtggtgagaaagggaaggctgagggggctattaaagaagttagcgagttgttggataagctggtaacaactgtaaagacagctgagggggcttcaaatggtactgatgcaattggaaaagttgtggataataataatgctgcgaaggctgctgataaggcgagtgtgacggggattgctaaggggataaaggagattgttgaagctgctggggggagtagtgaaaagctgaaagctgttgctgctgctaaaggggagagcaatgaaaaggcagggaagttgtttgggaaggctggtgctgctgctggggacagtgaggctgctagcaaggcggctggtgctgttagtgctgttagtggggagcagatattaagtgcgattgttaaggctgctggtgcggctgatcaggagggaaagaagcctgaggatgctaaaaatccgattgctgctgctattggggataaagatgggggtgcggagtttaatcatgagatgaagaaggatgatcagattgctgctgctattgctttgagggggatggctaaggatggaaagtttgctgtgaagagtggtggtggtgagaaagagaaggctgagggggctattaaagaagttagcgagttgttggataagctggtaaaagctgtgtaaagacagctgagggggcttcaagtggtactgatgcaattggagaagttgtggctgataatagtgctgcgaaggctgctgatgaggcgagtgtgacggggattgctaagggaataaaggagattgttgaagctgctggggggagtgaaaagctgaaagttgctgctgctgcaggggagaataataaaaaggcagggaagttgtttgggaaagttgataatgctaatgctggggacagtgaggctgctagcaaggcggctggtgctgttagtggggagcagatattaagtgcgattgttaaggctgctggtgaggctgagcaggatggagagaagcctggggaggctacaaatccgattgctgctgctattggaaagggtgatgaggatgcggattttggtaatgagatgaagaaggatggaaagtttgctttgctaactttctaagttctaagttactaggacagacaatcattcactactaaagattcacaagatttattttaaatagttcttaccttgattttatttttttataaattaagaaaatagaatactcttttaaaaaagaattc

SEQ ID NO: 116 - DNA, 1071 bp, Borrelia burgdorferi
atgaaaaaaatttcaagtgcaattttattaacaactttctttgtttttattaattgtaaaagccaagttgctgataaggacgacccaacaaacaaattttaccaatctgtcatacaattaggtaacggatttcttgatgtattcacatcttttggtgggttagtagcagaggcttttggatttaaatcagatccaaaaaaatctgatgtaaaaacctattttactactgtagctgccaaattggaaaaaacaaaaaccgatcttaatagtttgcctaaggaaaaaagcgatataagtagtacgacggggaaaccagatagtacaggttctgttggaactgccgttgagggggctattaaggaagttagcgagttgttggataagctggtaaaagctgtaaagacagctgagggggcttcaagtggtactgctgcaattggagaagttgtggctgatgctgatgctgcaaaggttgctgataaggcgagtgtgaaggggattgctaaggggataaaggagattgttgaagctgctggggggagtgaaaagctgaaagctgttgctgctgctaaaggggagaataataaaggggcagggaagttgtttgggaaggctggtgctgctgctcatggggacagtgaggctgctagcaaggcggctggtgctgttagtgctgttagtggggagcagatattaagtgcgattgttacggctgctgatgcggctgagcaggatggaaagaagcctgaggaggctaaaaatccgattgctgctgctattggggataaagatgggggtgcggagtttggtcaggatgagatgaagaaggatgatcagattgctgctgctattgctttgagggggatggctaaggatggaaagtttgctgtgaaggatggtgagaaagagaaggctgagggggctattaagggagctgctgagtctgcagttcgcaaagttttaggggctattactgggctaataggagacgccgtgagttccgggctaaggaaagtcggtgattcagtgaaggctgctagtaaagaaacacctcctgccttgaataagtga

SEQ ID NO: 117 - protein, 356 aa, Borrelia burgdorferi
MKKISSAILLTTFFVFINCKSQVADKDDPTNKFYQSVIQLGNGFLDVFTSFGGLVAEAFGFKSDPKKSDVKTYFTTVAAKLEKTKTDLNSLPKEKSDISSTTGKPDSTGSVGTAVEGAIKEVSELLDKLVKAVKTAEGASSGTAAIGEVVADADAAKVADKASVKGIAKGIKEIVEAAGGSEKLKAVAAAKGENNKGAGKLFGKAGAAAHGDSEAASKAAGAVSAVSGEQILSAIVTAADAAEQDGKKPEEAKNPIAAAIGDKDGGAEFGQDEMKKDDQIAAAIALRGMAKDGKFAVKDGEKEKAEGAIKGAAESAVRKVLGAITGLIGDAVSSGLRKVGDSVKAASKETPPALNK

SEQ ID NO: 118 - DNA, 936 bp, Borrelia burgdorferi
cgcaacgatgtttttggggtgcaaatagcagcagctttaaaaaatgtgtttgctattgcatttggaattttggatgcctataaattgaattatcctaatttaataggtaataatacagaatcatttttattttcaatatccttaaataatataaaagatattgcaatggagcttgggggaagaaatattgaaacgtttttatttttgtctggttctggcgatttagatgttacttgtagaagcatgtttggaagaaatagacgatttggcctaatagcattggaatgatagttatattcacaacaataagggttccaagctttattatggctgaagcatttttatcctttttaggacttggaatttcagctccaatgacaagctggggagaattagtgcaaaatggaattgctacatttgttgaatatccatggaaagtttttattccagctatagttatgacaatatttctattatttatgaactttttaggtgatgggctaagggatgcttttgatccaaaagatagcatctaaggagaaattgaatggaaaaagaaaatatattggaaataaaaaatttaacaattgaatttagattaaaacatacaacaattcatcccgtaagcaatgttaacctatctgtaaaaagaggagaaattagagctattgttggagaatctggaagcggaaaatccgtaacaagtatggctattttaaaattattaccagaacttacaacagtatataaaagtggagaaatactatttgaaaatcaagatctgctaaaacttagcgaaaaagaacttttaaaaatcagagggaataaaatatcaatgatatttcaagacccaatgacttcattaaacccatttttaagaatatcaactcaacttgaagaaacaataatcttacaccaaggattagggaaaaaaga

SEQ ID NO: 119 - protein, 95 aa, Borrelia burgdorferi
RNDVFGVQIAAALKNVFAIAFGILDAYKLNYPNLIGNNTESFLFSISLNNIKDIAMELGGRNIETFLFLSGSGDLDVTCRSMFGRNRRFGLIALE

SEQ ID NO: 120 - DNA, 1092 bp, Borrelia burgdorferi
atgagtttttataaggttatagggggagtatttatgaaaatatcagtaataggggcaggtgcttggggaacagctatttcaaagtctttggcagataaatttgattttaatatttttttatgggtctttgaagaagatgttaagaatgatattaataatgataatgttaatactaaatatttaaagggaattaaattgccaaaaaatttagttgcaagttcagatttatttgaagttgtaacaatgtctgattatattttcattgcaacaccttctctttttaccgttgatattttaaaaaaattggatcaatttttacattttctggagataaaaccaaagctagcaatacttacaaaagggtttattacttttgatggtaaaactcagacagttattgaagctgctgagagaattatgaaaggatataaagacgaaattacttatattgttggtccaagtcatgctgaggaagttgggcttggtgtgataacagggcttgttgcggctagtaataacagagaaaatgcatatttgtttattaatttatttagtaaaaccccaatttctttattttatagcaacgatgtttttggggtgcaaatagcagcagctttaaaaaatgtgtttgctattgcatttggaattttggatgcctataaattgaattatcctaatttaataggtaataatacagaatcatttttattttcaatatccttaaataatataaaagatattgcaatggagcttgggggaagaaatattgaaacgtttttatttttgtctggttctggcgatttagatgttacttgtagaagcatgtttggaagaaatagacgatttggcaatgaaattgttagcaaaaacattttagaaagctttttaagtatagatgacttgataagtaatattgaaaaaattggatatttaccagagggagttttggctgctaaatcaatttttttcttttttaaacaattaaatcgtgatctcaatcctaatagtttgttaagcgttatatataaaattttgaataaagagttggagcccaaatctgttattgagtatatgagagatgttagacaataa

SEQ ID NO: 121 - protein, 363 aa, Borrelia burgdorferi
MSFYKVIGGVFMKISVIGAGAWGTAISKSLADKFDFNIFLWVFEEDVKNDINNDNVNTKYLKGIKLPKNLVASSDLFEVVTMSDYIFIATPSLFTVDILKKLDQFLHFLEIKPKLAILTKGFITFDGKTQTVIEAAERIMKGYKDEITYIVGPSHAEEVGLGVITGLVAASNNRENAYLFINLFSKTPISLFYSNDVFGVQIAAALKNVFAIAFGILDAYKLNYPNLIGNNTESFLFSISLNNIKDIAMELGGRNIETFLFLSGSGDLDVTCRSMFGRNRRFGNEIVSKNILESFLSIDDLISNIEKIGYLPEGVLAAKSIFFFFKQLNRDLNPNSLLSVIYKILNKELEPKSVIEYMRDVRQ

SEQ ID NO: 122 - DNA, 211 bp, Borrelia burgdorferi
cttaaaatttcaagggcacctatttcgtttggatttgtttttaatagggaaattgctctttcgattaattttcgcgttctctcatcatcttttttgattttccctatatttttacctattgtaagttttaatgctattttaaaaaataaaaacactaaaataaatactgtcaatgaagagattactataaatagtgacaacactcgtattg

SEQ ID NO: 123 - protein, 70 aa, Borrelia burgdorferi
LKISRAPISFGFVFNREIALSINFRVLSSSFLIFPIFLPIVSFNAILKNKNTKINTVNEEITINSDNTRI

SEQ ID NO: 124 - DNA, 727 bp, Borrelia burgdorferi
cgaattaatttttttattttatttttttataataaaataagtccagcattagtaattatacttagtggatttttttatacattaatataaatatgattaaacaaaaattaaaaatatctcaaaatttaaactcaattcaaatacaaacaataaaaatattaagccttaaccaaaaagaattaacaaagcttatactagaagaaagcgaaaataatgaatgtctagaaataaactcaaataaaatattttttgaaacattgaaaacatataggtttaaaaaagttttttataaagaagatgatatgataaaaaatcaacacgacatagctcttgaaaaaacacaaacaaatacttctttaaaagaacaccttttactgcaattaagaattcaaagaataaatgaagatgaaattaaaataggcgaaatactcataaacaatctaaacagcaaaggttttcatataataaacccttacgatctttttaaaaaggaagaaaaagaaaaagtaaaaaaaataattgaacttattcaaaaatttgatccaattggaatttgtgtccccaacataatagaatcgttaattttgcaagcaaagcatcataaattagaaactaatattattaaaattcttgaaaaagcagagcttcttgaaaaaactcaaaaaaagttaaaagaggaacttaaaataagaagcaaagaatttaacacggctttagaaattatcag

SEQ ID NO: 125 - protein, 29 aa, Borrelia burgdorferi
RINFFILFFYNKISPALVIILSGFFYTLI

SEQ ID NO: 126 - DNA, 534 bp, Borrelia burgdorferi
ttgattttaataaatttattcattacattcttaaaaatcggattattaaatttcggaggcggtaatggaattgcagcaataataaacaacgaaataattaataataaacattggataacaaaagaagaatttgtcaatatgattacaatatcaagaataacccctgggcctattgcaacaaacatagcaacatacgttggaatgaaaactgcaggaattgcgggagcaataattgctacagtagcattaataacagccccaataataataatgattataatcctcctaatactacataaaatcggctttttaaattattgcctagaaaatctaaaacctattattgttgcgctgtggataattacaataatcattttgcttgaaaatacatatttaaaaatagaaaacaacaaaacagaacttttgaaaactctggctattgtaggaattaatttttttattttatttttttataataaaataagtccagcattagtaattatacttagtggatttttttatacattaatataa

SEQ ID NO: 127 - protein, 177 aa, Borrelia burgdorferi
MILINLFITFLKIGLLNFGGGNGIAAIINNEIINNKHWITKEEFVNMITISRITPGPIATNIATYVGMKTAGIAGAIIATVALITAPIIIMIIILLILHKIGFLNYCLENLKPIIVALWIITIIILLENTYLKIENNKTELLKTLAIVGINFFILFFYNKISPALVIILSGFFYTLI

SEQ ID NO: 128 - DNA, 213 bp, Borrelia burgdorferi
cctagtaatatatcaacaatgaaaaaatacttaaatcaattagaaaaagaaataaaaatcatagcaaaattctattttaaaaacgatcaatctctaatttattgcaaacttaattataccctagaaaaaatttgtttaaaactaataaaattctacaaaaaattctacaaagaattaaaacaatttacacaaaagaacattactacttaattg

SEQ ID NO: 129 - protein, 69 aa, Borrelia burgdorferi
PSNISTMKKYLNQLEKEIKIIAKFYFKNDQSLIYCKLNYTLEKICLKLIKFYKKFYKELKQFTQKNITT

SEQ ID NO: 130 - DNA, 444 bp, Borrelia burgdorferi
ttgggaaattatcaatacttttttaatttattctttattttcaaaataatctttatatacttatatattatgtataaggctataaaagaacaacaagaaatagaaatagatcatgcatgcagaatacttattcttaccgcaacaatatttgaaataaattcaatattcgaaaattattatcaaaaaactctactcaaaaagtataacgaaaatctcaaaaacaaaaatctacctcctagtaatatatcaacaatgaaaaaatacttaaatcaattagaaaaagaaataaaaatcatagcaaaattctattttaaaaacgatcaatctctaatttattgcaaacttaattataccctagaaaaaatttgtttaaaactaataaaattctacaaaaaattctacaaagaattaaaacaatttacacaaaagaacattactacttaa

SEQ ID NO: 131 - protein, 147 aa, Borrelia burgdorferi
MGNYQYFFNLFFIFKIIFIYLYIMYKAIKEQQEIEIDHACRILILTATIFEINSIFENYYQKTLLKKYNENLKNKNLPPSNISTMKKYLNQLEKEIKIIAKFYFKNDQSLIYCKLNYTLEKICLKLIKFYKKFYKELKQFTQKNITT

SEQ ID NO: 132 - DNA, 921 bp, Borrelia burgdorferi
cacataaaagaaatagacctaggtatttatcaacagtatgtaaattagttgcttgatgctaaaaaatcgatattaggagagtttaatgtctaatgttactaccatagagaaattagatagtattttacaagaagataagtggacaagaatagttgttaataattattctcttgccaaaataaaagaattggatgatttaataaataatataattgatgaggggttaacagaggatgtgcttgatatttgtggcaggcatttaaaggacgttaaaaaaagcatagcaggtctttatatttctggaatgcttatatatagtaggcgacctttaaatgatatgaatttacttgctgttattgatttgttttctcaaaatttaaaatgggctcttgttgagcatatatgtaatgaaatgcttttaatttctgagaataagcacgctctttatactcttgcaaaaatatatgctcagaacaatgaaaatgataagcttccaggtatttggactagaattgttgaagcagatattgacgacactgtgtttgttaggcaacttgctacttattatgaaactattgatttgcaaaaatcaattttctattttagaaaagctatttatcgttttattgataaaaaacaaatgtcaggaatcagagaagtatgggccaagcttattcattatgtttcagatgattttgattcttttttgcttattttgcaaaaaattgaaaaagatcttggatttaaaaaggccatagttctttatgaggatttatttgatcattattctttgactgacaatgttgacgagacaatagaaattttaaaaggcattttaaaacttgataacaaaaatcataaggcaagagaaaatttagttaaatttttaagagaaagatataaagatgttaaaaatattga

SEQ ID NO: 133 - protein, 15 aa, Borrelia burgdorferi
HIKEIDLGIYQQYVN

SEQ ID NO: 134 - DNA, 1005 bp, Borrelia burgdorferi
gtgtcatcagaaagaatcgttgaattgattaaagagatttatttagattttagaaaaggcaatttcaaagaagctttagtaaaatccgaagaggctcattctcttgattttgataatattgaaattctaacagctttagtaaaatccgaagaggctcattctcttgattttgataatattgaaattctaacagctttgaaaagttctgtgtattggaatggtcaagttgaaagccttgatagaatagggcaagattatgaaaaggcagaatttttaataagagagtggaataattttgcaggacgatatttgaaaaagatgggttgtaattttttgcaaggtcgcaattctataaaatattttgtgtttcagacctgtctttatatatataaaaatatatacaaaatgcatccagaaaatttggaccttttaataaaaattgctaagtcttataagggtatgggaaattacgagagagcaatcaatgtttttttgcaaatattaggagattttaaagataactcagatgttgttgcagagcttgctgattcttatgcgttggttgatgaaattaaagaagctaaagtattattcagagaggctttttttattaatcctcaaaagatagatatatattctcttgagtctgatatgattttaagattaatagatcttattaagtctgacagaaatatttcagatgatcttataaaagaatggattcctgtttatggctctcttaatggtgtttttaatattaaaagagagttaaggcctatagagcttggacagctaaagcagtctgtttacagtttgcgcaatgagcttaaagaaaagtcttatagatcaataaatgagagcatattgcttccaaggcttattaataaatatttttggcttattgatcattatgtaaggataaaagaagatagagcacgtattgatgagattttgttatacataaaagaaatagacctaggtatttatcaacagtatgtaaattag

SEQ ID NO: 135 - protein, 314 aa, Borrelia burgdorferi
MSSERIVELIKEIYLDFRKGNFKEALVKSEEAHSLDFDNIEILTALKSSVYWNGQVESLDRIGQDYEKAEFLIREWNNFAGRYLKKMGCNFLQGRNSIKYFVFQTCLYIYKNIYKMHPENLDLLIKIAKSYKGMGNYERAINVFLQILGDFKDNSDVVAELADSYALVDEIKEAKVLFREAFFINPQKIDIYSLESDMILRLIDLIKSDRNISDDLIKEWIPVYGSLNGVFNIKRELRPIELGQLKQSVYSLRNELKEKSYRSINESILLPRLINKYFWLIDHYVRIKEDRARIDEILLYIKEIDLGIYQQYVN

SEQ ID NO: 136 - DNA, 307 bp, Borrelia burgdorferi
ctatttattgtttttgaagtttactctaagccagcttttataagtcaagacgattcgtatgagcttgattttagtagtggagaggtagatattagtgtaaataccaattcaaaatttaatctttcttttaaagatgagtcttggatttatatcaaaagcattgaaaatgaagcttttattaagttaattggagaatcttatgataacggtgctgtttttacttttcagacttttaaaaaagaaggcaaaattaaattggttttcacttatcaaaatgttaaagattcaagtgaatttaataaaataa

SEQ ID NO: 137 - protein, 102 aa, Borrelia burgdorferi
LFIVFEVYSKPAFISQDDSYELDFSSGEVDISVNTNSKFNLSFKDESWIYIKSIENEAFIKLIGESYDNGAVFTFQTFKKEGKIKLVFTYQNVKDSSEFNKI

SEQ ID NO: 138 - DNA, 1041 bp, Borrelia burgdorferi
atgattagaaaatatttgatttatataagtttgctatttattgtttttgaagtttactctaagccagcttttataagtcaagacgattcgtatgagcttgattttagtagtggagaggtagatattagtgtaaataccaattcaaaatttaatctttcttttaaagatgagtcttggatttatatcaaaagcattgaaaatgaagcttttattaagttaattggagaatcttatgataacggtgctgtttttacttttcagacttttaaaaaagaaggcaaaattaaattggttttcacttatcaaaatgttaaagattcaagtgaatttaataaaataattatcttgaaaattacaaagaattttgaagttgcaattccacaaggcgttggtggtggctctagcagggacaataacattgaaactggtaataatcttgaacttgggggggggagtattagcggggcaacttctaaagagattattgttagggctttaaatttgtcctacataaatgattacaaaggagcaatagatttgcttaataagtataatttcaatgacgataaatatattttattgaaggcggaaattcattataaaaatggtgattatttaaaatcttatgaaaattatttgaaattgaagagtaaatattttcaaagcattgtttttgatctaattaggcttgctatagaattaaatattaaagaagaggttttagagaacgctagatatttagttgaaaagaatgttgatttttctgagagcatttatcttgagatctttgaattcttagtaacaaggggagagcatgagtttgctttaaattttagctctctttactttcctaagtatattaattcaagcttttcagacaaatatagttatttgttgggaaaactttatgagtctgagagcaagcataaagattttttaaaggctttgcattactataaattggttattgataattacccttttagttattattatgagagagccaagataagatatttatttttaaagcggtttttttag

SEQ ID NO: 139 - protein, 346 aa, Borrelia burgdorferi
MIRKYLIYISLLFIVFEVYSKPAFISQDDSYELDFSSGEVDISVNTNSKFNLSFKDESWIYIKSIENEAFIKLIGESYDNGAVFTFQTFKKEGKIKLVFTYQNVKDSSEFNKIIILKITKNFEVAIPQGVGGGSSRDNNIETGNNLELGGGSISGATSKEIIVRALNLSYINDYKGAIDLLNKYNFNDDKYILLKAEIHYKNGDYLKSYENYLKLKSKYFQSIVFDLIRLAIELNIKEEVLENARYLVEKNVDFSESIYLEIFEFLVTRGEHEFALNFSSLYFPKYINSSFSDKYSYLLGKLYESESKHKDFLKALHYYKLVIDNYPFSYYYERAKIRYLFLKRFF

SEQ ID NO: 140 - DNA, 15 bp, Borrelia burgdorferi
gatctggatccaggc

SEQ ID NO: 141 - DNA, 11 bp, Borrelia burgdorferi
gcctggatcca

(19) United States
(12) Patent Application Publication
(10) Pub. No.: US 2005/0058661 A1
(43) Pub. Date: Mar. 17, 2005

(54) Methods and compositions for vaccination comprising nucleic acid and/or polypeptide sequences of the genus Borrelia

(76) Inventors: Kathryn F. Sykes, Dallas, TX (US); Katherine S. Hale, Dallas, TX (US); Stephen A. Johnston, Dallas, TX (US)

Correspondence Address: FULBRIGHT & JAWORSKI L.L.P., 600 CONGRESS AVE., SUITE 2400, AUSTIN, TX, 78701, US

(21) Appl. No.: 10/688,058

(22) Filed: Oct. 17, 2003

Related U.S. Application Data
(60) Provisional application No. 60/419,401, filed on Oct. 18, 2002.

(51) Int. Cl. A61K 39/02; A61K 48/00

(52) U.S. Cl. 424/190.1; 424/234.1; 514/44

(57) ABSTRACT

The invention relates to antigens and nucleic acids encoding such antigens obtainable by screening a Borrelia genome, in particular an B. burgdorferi genome. In more specific aspects, the invention relates to methods of isolating such antigens and nucleic acids and to methods of using such isolated antigens for producing immune responses. The ability of an antigen to produce an immune response may be employed in vaccination or antibody preparation techniques.

This application claims priority to U.S. Provisional Application No. 60/419,401 filed Oct. 18, 2002.

The government owns rights in the present invention pursuant to DARPA Grant # MDA9729710013.

### BACKGROUND OF THE INVENTION

1. Field of the Invention

The present invention relates generally to the fields of vaccinology, immunology, bacteriology, virology and molecular biology. More particularly, the invention relates to methods for screening and obtaining vaccines generated from the administration of expression libraries constructed from a Borrelia burgdorferi (B. burgdorferi) genome. In particular embodiments, it concerns methods and compositions for the vaccination of a subject against B. burgdorferi infections, wherein vaccination of the subject may be via compositions comprising polypeptides or polynucleotides or variants thereof, derived from part or all of the genes or similar sequences performing as vaccines.

2. Description of Related Art

Purely on empirical grounds, Edward Jenner first demonstrated protective vaccination against infectious disease in the 1790s. After observing that milkmaids did not contract smallpox, he intentionally infected a boy with cowpox then subsequently found him immune to smallpox. Since then, vaccines against measles, polio, anthrax, rabies, typhoid fever, cholera and the plague, have been developed. The methods of developing new vaccines vary and differ for each virus, bacteria, or other pathogen target; however, they have traditionally consisted of whole pathogens in an attenuated or killed form, as did Jenner's vaccine. Both social and economic considerations make vaccination the optimal method for protecting animals and humans against disease or death. However, vaccines have not been developed for many of the most serious human diseases, including Malaria, tuberculosis, HIV, respiratory syncytial virus (RSV), Streptococcus pneumoniae, rotavirus, Shigella and other pathogens. There is a need to develop effective vaccines, yet for many pathogens vaccines are not readily produced. For example, the antigenic drift of influenza virus requires that new vaccines be constantly developed. Research efforts continue to try to identify effective vaccines for rabies (Xiang et al., 1994), herpes (Rouse, 1995); tuberculosis (Lowrie et al., 1994); HIV (Coney et al., 1994) as well as many other diseases or pathogens.

Most currently available vaccines are composed of live/attenuated pathogens (Ada, 1991). These live inocula infect cells and elicit a broad immune response in the host. The strength of this approach is that no antigen identification is required, because all the components of the pathogen are presented to the immune system. However, this straightforward approach carries an inherent problem. Pathogenicity of the attenuated strain or reversion to virulence is possible. At best, components of the pathogen that are not needed for the protective immune response are carried as baggage; alternatively some components may compromise protective immunity. Pointedly, pathogens become pathogenic by evolving or acquiring factors to defend themselves against or avoid a host immune system. In whole organism vaccines, the repertoire of antigens and their expression levels are controlled by the pathogen. Consequently, the host immune system is often not directed to the most protective antigen determinants. Another consideration is that presentation of all antigens of a pathogen provides opportunities for the unprotective ones to cause deleterious side effects such as autoimmunity or toxicity.

Alternatives to the use of live/attenuated pathogen vaccines include the use of pathogen components for production of immune responses such as antibodies to single antigens or to a limited number of antigens associated with a pathogen or disease. Recombinant subunit or peptide vaccines are comprised of only a single or small number of pathogen components. They have provided improved immunogenicity, reduced side-reactivities and easier quality control than whole organism vaccines. However, the antigens conferring the best protection are usually unknown, so the choice has often fallen to educated guessing or technical convenience, followed by experimentation. For example, subunits have been tested as vaccines that correspond to components of the pathogen that i) generate high levels of antibodies, ii) are expressed on the pathogen surface or are secreted, iii) carry consensus major histocompatibitilty (MHC) binding sites, or iv) are abundant and easy to purify. Unfortunately these candidates must be unsystematically tested by trial and error, since broad-based functional screens for vaccine candidates are impractical using protein, peptide, or live vector delivery methods. This defines a more basic and unsolved problem of identifying the particular gene or genes of the pathogen that will express an immunogen capable of priming the immune system for rapid and protective response to pathogen challenge.

However, despite promising initial results with genetic vaccination, there remains the more basic and unsolved problem of identifying the particular gene or genes of the pathogen that will express an immunogen capable of priming the immune system for rapid and protective response to pathogen challenge. Certain non-viral pathogens and some viruses have very large genomes. Protozoa genomes contain up to about 108 nucleotides that can encode more than 5,000 genes, thus posing an expensive and time-consuming analytical challenge to identify or isolate effective immunogenic antigens. Evaluating the immune potential of the millions of possible determinants from even one pathogen is a significant hurdle for new vaccine development.

A comprehensive, unbiased approach to antigen selection for a subunit vaccine is enabled by combining genetic immunization (Tang et al., 1992) with the invention of expression library immunization (ELI) (Barry et al., 1995). ELI is an empirical method, as was Jenner's, to identify protective vaccines, however, unlike Jenner's is based on a subunit rather than whole pathogen endproduct. Using ELI, the entire genome of a pathogen can be searched for protective antigens. Pathogen DNA is fragmented and cloned into a mammalian expression vector to generate a library corresponding to all of the genetic material of the organism. In 1995 the utility of ELI was demonstrated in the protection of mice against Mycoplasma pulmonis challenge by vaccination with a pathogen library. The complete library is partitioned into sub-libraries that are used to separately immunize groups of test animals. Sub-library inocula that protect animals from disease following challenge are scored as positive. Presumably one or more plasmids within a positive sub-library are responsible for the protective response. To identify the constituent antigen-expressing plasmid(s) that holds protective capacity, the sub-libraries can be further subdivided and tested. Plasmid DNA is prepared from the pools and used to inoculate more test animals, which are assayed for protection. Other researchers have subsequently reported the successful application of ELI against other bacterial and parasitic pathogens. Brayton et. al. used a Rickettsia (Cowdria ruminantium) expression library to screen for protective sub-library pools in a murine model of Heartwater disease. Four out of ten groups of mice inoculated with different sub-libraries and challenged with an optimal level of bacteria showed reduced levels of infection (Brayton et al., 1998). In another experiment, a partial expression library was made from cDNA of the parasitic helminth Taenia crassiceps and used to immunize mice against cysticerosis disease. Though the inoculum only represented a portion of the genome, a two-fold reduction in parasitemia was observed (Manoutcharian et al., 1998). Alberti et. al. found that an expression library made from the genome of Trypanosoma cruzi (a protozoa that causes Chagas' disease) stimulated specific immune responses in mice (Alberti et al., 1998). Finally a library made from the genomic DNA of Leishmania major (a protozoa that causes leismaniasis) was able to marginally reduce parasite load in challenged mice (Piedrafita et al., 1999). Test mice inoculated with further sub-divisions of this library displayed greater levels of protection than the original. This indicates that the protective clone(s) was being enriched through two rounds of reduction in the complexity of the plasmid inocula.

In particular, new protective antigens need to be discovered for pathogens of the genus Borrelia. Within the U.S., Borreliosis, or Lyme disease accounts for 95% of the vector-borne illnesses according to the Center for Disease Control and Prevention (CDC). Ticks (Ixodidae family) the primary vector for Borrelia dissemination, transmit more disease to the United States and European populations every year than any other vector (Rahn, 2001). Screening and identification of a particular gene or genes that will express an immunogen capable of priming the immune system for rapid and protective response to Borrelia challenge would improve human health.

The etiologic agent of Lyme disease is a spirochete bacterium of the Borrelia genus. Borrelia burgdorferi predominates in the U.S. but Borrelia garinii and Borrelia afzelii, as well as others, are common in Europe (Rahn, 2001). Human infection occurs through a zoonotic route. The white-footed mouse and the white-tailed deer serve as bacterial reservoirs in the U.S., since they are favored sources of blood meal for the deer-tick (Ixodes scapularis). Transmission of the Borrelia spirochete to humans occurs following a bite from an infected tick (Gayle and Ringdahl, 2001). In 1990, less than 8,000 U.S. cases of Lyme disease were reported to the CDC. However by 1999 the number had jumped to 16,273 cases (Gayle and Ringdahl, 2001). Endemic areas, mostly in the northeastern, mid-atlantic, and north-central states, suffer incidence levels of 1% to 3% of the population, according to the CDC. The namesake of the disease comes from the town of Lyme, Conn.; in which a cluster of infections surfaced as juvenile rheumatoid arthritis cases in 1975 (Thanassi and Schoen, 2000). While the disease is geographically focused, surveys show that incidence is spreading. Demographically, children under 15 years of age and adults over 30 show the greatest number of infections. It has been estimated that from seven-fold to twelve-fold more infections than reported occur but are undiagnosed (Van Solingen and Evans, 2001). If the infecting tick bite is not noticed then the subsequent illness can be difficult to identify as Lyme disease because of the variability of initial symptoms and lack of serological testing standards. It has three stages that begin days to weeks following a tick bite and is characterized by an expanding skin lesion, and is sometimes accompanied by flu-like symptoms. Approximately 60% of infected individuals develop intermittent episodes of arthritis several weeks after the bite (Thanassi and Schoen, 2000). The rash and the initial arthritis resolves in a few days or weeks, however if untreated the spirochetes spread to other sites such as the host central nervous system, heart, or joints. Treatment of early stage infection with antibiotics such as amoxicillin or doxycycline usually results in the return of an individual to normal health; however later treatment is less effective in eliminating disease. Antimicrobial therapy of disseminated Lyme Borreliosis for as much as three months may not be sufficient to eliminate spirochetes or prevent relapses (Hercogova, 2001 and Steere et al., 2001). During the middle stage, the inflammatory manifestations of the disease develop into meningitis, cardiac blockage, or arthritis. In late stage disease months or years following initial infection, spirochetes are usually not detectable but malaise continues. This may consistent of chronic arthritis, neurologic abnormalities, acrodermatitis chronica atrophicans, or other complications (Komacki and Oliver, 1998). Infection with B. burgdorferi also causes moderate to severe arthritis in dogs, hamsters, mice, monkeys, and rats (Poland and Jacobson, 2001 and Croke et al., 2000). It is hypothesized that symptoms are a consequence of a continued host immune response either to the cleared bacterium or against a tissue autoantigen. Borrelia mimicry of a self-antigen has been shown to activate this T-cell mediated immunopathology that is perpetuated (Trollmo et al., 2001). A particular HLA (-DR4) subtype, which is found in a third of the population, has been correlated with individuals that develop persistent arthritis (Rahn, 2001). The proposed autoimmune mechanism has implications for the utility and safety of a Lyme vaccine. For example, any vaccine that engenders a host immune response that resembles those responses stimulated by a Borrelia infection might cause disease. An additional consideration for vaccine design is that previous infection does not appear to prevent reinfection, indicating that long-term immunity is not engendered by the whole bacterium (Rahn, 2001).

In patients infected with B. burgdorferi, a complex array of cellular and humoral immune responses to a variety of antigens are induced (Vaz et al., 2001). Early research toward a Lyme vaccine focused on using whole-pathogen formulations. Inactivated whole-cell lysates were shown to protect hamsters and dogs against spirochetemia, but appeared to mediate large-joint arthritis. Subsequent investigation identified B. burgdorferi antibodies that cross-reacted with host nerve cell axons, synovial cells, hepatocytes, and cardiac muscle proteins. The Borrelia antigens believed responsible for inducing the host self-reactivity are a flagellin subunit, heat shock proteins, and LFA-1α (Trollmo et al., 2001 and Wormser, 1996). Due to the concern of vaccine-induced autoimmunity, the development of a human Lyme disease has focused on a subunit rather than whole-cell design, see description below. Vaccination with several outer surface proteins has conferred at least some level of protection in animal models (Wormser, 1996; Fikrig et al., 1990; and Gerber, 1999). These subunits include OspA, OspB, OspC, and the 39 kDa protein. A decorin-binding protein has also been studied in mice (Hagman et al., 1998). Of this group of antigens, OspA emerged as the leading Lyme borreliosis vaccine candidate.

The current FDA licensed vaccine, LYMErix, is comprised of recombinant OspA. Grown in culture, B. burghdorferi expresses predominantly two proteins: i) the flagellin subunit indicated in autoimmunity, and ii) the species-specific lipoprotein, outer surface protein A (OspA). Despite the apparent abundance and surface exposure of OspA, individuals naturally infected with Borrelia do not develop high titers of anti-OspA antibodies. Determination of the spirochete's life cycle showed that the bacterium down-regulates OspA expression as it leaves the tick and enters the mammalian host (Straubinger et al., 2002). Consequently an OspA-based vaccine must operate by inactivating the pathogen within the tick mid-gut, and therefore is dependent on transfer of sufficient quantities of active antibodies from host to tick. Nonetheless, the anti-OspA vaccine has been shown to be protective in a number of animal models. The year-long regimen for human administration was designed with a schedule of three immunizations to generate anti-OspA-mediated borreliacidal antibody responses, although these titers have been shown to rapidly wane (Jensen et al., 1998). Another drawback of an OspA (or Osp B or OspC) based vaccine is the heterogeneity of the protein among isolates of B. burgdorferi in nature. Challenges of OspA immunized mice with homologous isolates have been protective, but challenges with diverse isolates have not been successful (Wormser, 1996). Phase III clinical trials were considered successful in demonstrating 76% overall efficacy in preventing infection during two seasons of lyme disease transmission. LYMErix was approved and available from December 1998 (Thanassi and Schoen, 2000) until February 2002. In sum, a more efficacious vaccine than LYMErix can be envisioned and there are no vaccines currently marketed.

The mechanism of immune action appears to be the production of high-titer antibodies specific for a conformational epitope of OspA from B. burgdorferi sensu lato. After LYMErix was released, it was shown that yearly boosters, following the three-dose immunization series, are required to maintain antibodies at adequately high levels (Thanassi and Schoen, 2000). The randomized vaccine efficacy trial was tested where only B. burgdorferi sensu lato is found. The ability of LYMErix to cross-protect against the heterogeneous subspecies and different Borrelia species is unknown. An experiment in mice with an OspA carrying a small number of amino acid changes showed no cross-protection. Another concern is that the highest risk group, children under 15, is not approved to receive this vaccine (Poland and Jacobson, 2001). Although vaccine recipients reported no unusual levels of arthritis during the 20-month phase III trial, several case studies subsequent to the report have raised concerns of vaccine-induced molecular mimicry (Rose et al., 2001). Chronic Lyme arthritis has been associated with increased OspA reactivity in synovial fluid. Evidence has been presented that recombinant OspA priming can induce severe destructive arthritis in hamsters after spirochete infection (Croke et al., 2000). The removal of LYMERrix from the market this year occurred because of poor sales, which may be attributed to public concern over long term efficacy and possible adverse autoimmune effects from the OspA antigen. Currently, no Lyme vaccine is commercially available.

More recently, the tertiary structure of the OspA protein has been studied with the idea of designing a more broadly protective version of the variable antigen (Luft et al., 2002). However whether the cited problems are real or perceived, the development of a new product that is both more effective and publicly accepted is likely to require a non-OspA composition. The rationale for having a vaccine is the documented increase in Lyme disease incidence, the geographic spread of the disease, the success of re-infections, and the association of disease with permanent rheumatoid or neurological symptoms.

### SUMMARY OF THE INVENTION

The present invention overcomes various difficulties and problems associated with immunization against bacteria of the Borrelia genus. Various embodiments of the invention include compositions comprising Borrelia polypeptides and polynucleotides, which encode such polypeptides, that may be used as antigens for immunization of a subject. The present invention may also include vaccines comprising antigens derived from bacteria of the Borrelia genus, as well as methods of vaccination using such vaccines. Vaccine compositions and methods may be broadly applicable for immunization against a variety of Borrelia infections and the diseases and disorders associated with such infections. An antigen, as used herein, is a substance that induces an immune response in a subject. In particular, compositions and methods may include polypeptides and/or nucleic acids that encode polypeptides obtained by screening the genome of a bacterium or bacteria of the Borrelia genus, (e.g., Borrelia borgdorferi sensu lato and Borrelia afzelii).

Certain embodiments of the invention include isolated polynucleotides derived from members of the Borrelia genus. In some embodiments, polynucleotides may be isolated from bacteria of the genus Borrelia, in particular Borrelia burgdorferi or Borrelia afzelii, or any other member of Borrelia genus. Polynucleotides may include but are not limited to nucleotide sequences comprising the sequences as set forth in SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:101, SEQ ID NO:103, SEQ ID NO:105, SEQ ID NO:107, SEQ ID NO:109, SEQ ID NO:111, SEQ ID NO:113, SEQ ID NO:115, SEQ ID NO:116, SEQ ID NO:118, SEQ ID NO:120, SEQ ID NO:122, SEQ ID NO:124, SEQ ID NO:126, SEQ ID NO:128, SEQ ID NO:130, SEQ ID NO:132, SEQ ID NO:134, SEQ ID NO:136, or SEQ ID NO:138, a complement, a fragment, or a closely related sequence thereof. In additional embodiments, the invention may relate to such polynucleotides comprising a region having a sequence comprising at least 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 125, 150, 200, or more contiguous nucleotides in common with at least one of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:101, SEQ ID NO:103, SEQ ID NO:105, SEQ ID NO:107, SEQ ID NO:109, SEQ ID NO:111, SEQ ID NO:113, SEQ ID NO:115, SEQ ID NO:116, SEQ ID NO:118, SEQ ID NO:120, SEQ ID NO:122, SEQ ID NO:124, SEQ ID NO:126, SEQ ID NO:128, SEQ ID NO:130, SEQ ID NO:132, SEQ ID NO:134, SEQ ID NO:136, or SEQ ID NO:138, a complement, or fragment thereof, as well as any intervening lengths or ranges of nucleotides.

A Borrelia polynucleotide may be isolated from a genomic or episomal DNA expression library, but it need not be. For example, the polynucleotide may also be a sequence from one species that is determined to be protective based on the protective ability of a homologous sequence in another species. For example, the polynucleotide could be a sequence selected from a B. borgdorferi or B. afzelei that was determined to be protective after analysis of the respective genomic sequence(s) for other members of Borrelia or related organisms that had previously been shown to be protective in an animal or human subject. As discussed below, the polynucleotides need not be of natural origin, or to encode an antigen that is precisely a naturally occurring Borrelia antigen.

In many embodiments, a polynucleotide encoding a Borrelia polypeptide may be comprised in a nucleic acid vector, which may be used in certain embodiments for immunizing a subject against a member of the Borrelia genus (e.g., genetic immunization). In various embodiments a genetic immunization vector may express at least one polypeptide encoded by a Borrelia polynucleotide. In other embodiments, the genetic immunization vector may express a fusion protein comprising a Borrelia polypeptide. A polypeptide expressed by a genetic immunization vector may include a fusion protein comprising a Borrelia polypeptide, wherein the fusion protein may comprise a heterologous antigenic peptide, a signal sequence, an immunostimulatory peptide, an oligomerizing peptide, an enzyme, a marker protein, a toxin, or the like. A genetic immunization vector may also, but need not, comprise a polynucleotide encoding a Borrelia-polypeptide/mouse-ubiquitin fusion protein.

A genetic immunization vector, in certain embodiments, will comprise a promoter operable in eukaryotic cells, for example, but not limited to a CMV promoter. Such promoters are well known to those of skill in the art. In some embodiments, the polynucleotide is comprised in a viral or plasmid expression vectors. A variety of expression systems are well known. Expression systems include, but are not limited to linear or circular expression elements (LEE or CEE), expression plasmids, adenovirus, adeno-associated virus, retrovirus and herpes-simplex virus, pVAX1™ (Invitrogen); pCI neo, pCI, and pSI (Promega); Adeno-X™ Expression System and Retro-X™ System (Clontech) and other commercially available expression systems. The genetic immunization vectors may be administered as naked DNA or incorporated into viral, non-viral, cell-mediated, pathogen mediated or by other known nucleic acid delivery vehicles or vaccination methodologies.

In alternative embodiments, a polynucleotide may encode one or more antigens that may or may not be the same sequence. A plurality of antigens may be encoded in a single molecule an in any order and/or a plurality of antigens may be encoded on separate polynucleotides. A plurality of antigens may be administered together in a single formulation, at different times in separate formulations, or together in separate formulations. Polynucleotides may comprise at least 1, 2, 3, 4, 5, 6, 7, 8, 9, 10 or more polynucleotides or fragments thereof encoding at least 1, 2, 3, 4, 5, 6, 7, 8, 9, 10 or more antigens derived from one or more bacteria of the Borrelia genus, and may include other antigens or immunomodulators from 1, 2, 3, 4, 5, 6, 7, 8, 9, 10, or more other pathogens as well.

Various embodiments of the invention may include bacterial polypeptides, including variants or mimetics thereof, and compositions comprising bacterial polypeptides, variants or mimetics thereof. Bacterial polypeptides, in particular B. burgdorferi polypeptides, include, but are not limited to amino acid sequences set forth in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, SEQ ID NO:112, SEQ ID NO:114, SEQ ID NO:117, SEQ ID NO:119, SEQ ID NO:121, SEQ ID NO:123, SEQ ID NO:125, SEQ ID NO:127, SEQ ID NO:129, SEQ ID NO:131, SEQ ID NO:133, SEQ ID NO:135, SEQ ID NO:137, or SEQ ID NO:139, fragments, variants, or mimetics thereof, or closely related sequences. In additional embodiments, the invention may relate to polypeptides comprising a region having an amino acid sequence comprising at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 125, 150, 200, or more contiguous amino acids in common with at least one of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:10, SEQ ID NO:112, SEQ ID NO:114, SEQ ID NO:117, SEQ ID NO:119, SEQ ID NO:121, SEQ ID NO:123, SEQ ID NO:125, SEQ ID NO:127, SEQ ID NO:129, SEQ ID NO:131, SEQ ID NO:133, SEQ ID NO:135, SEQ ID NO:137, or SEQ ID NO:139, a complement, or fragment thereof, as well as any intervening lengths or ranges of amino acids. Additional embodiments of the invention also relate to methods of producing such polypeptides using known methods, such as recombinant methods.

Polypeptides of the invention may be synthesized, recombinant or purified polypeptides. Polypeptides of the invention may have a plurality of antigens represented in a single molecule. The antigens need not be the same antigen and need not be in any particular order. It is anticipated that polynucleotides, polypeptides and antigens within the scope of this invention may be synthetic and/or engineered to mimic, or improve upon, naturally occurring polynucleotides or polypeptides and still be useful in the invention. Those of ordinary skill will, in view of the specification, be able to obtain any number of such compounds.

Various embodiments of the invention include vaccine compositions. A vaccine composition may comprise (a) a pharmaceutically acceptable carrier; and (b) at least one antigen. In certain embodiments of the invention the vaccine may be against bacteria of the Borrelia genus. In other embodiments, a vaccine may be directed towards a member of the Borrelia genus and in particular B. burgdorferi sensu lato or other member of the burgdorferi group. In some embodiments, an Borrelia antigen has a sequence as set forth in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, SEQ ID NO:112, SEQ ID NO:114, SEQ ID NO:117, SEQ ID NO:119, SEQ ID NO:121, SEQ ID NO:123, SEQ ID NO:125, SEQ ID NO:127, SEQ ID NO:129, SEQ ID NO:131, SEQ ID NO:133, SEQ ID NO:135, SEQ ID NO:137, or SEQ ID NO:139, fragments, variants, or mimetics thereof, or closely related sequences.

In certain embodiments of the invention a vaccine may comprise: (a) a pharmaceutically acceptable carrier, and (b) at least one polypeptide and/or polynucleotide encoding a polypeptide having a Borrelia sequence, including a fragment, variant or mimetic thereof. Borrelia polypeptides and/or polynucleotides include, but are not limited to Borrelia polypeptides or polynucleotides; fragments thereof, or closely related sequences. In some embodiments a Borrelia polypeptide or polynucleotide may be a B. burgdorferi sequence.

The vaccines of the invention may comprise multiple polynucleotide sequences and/or multiple polypeptide sequences. In some embodiments, the vaccine will comprise at least a first polynucleotide encoding a polypeptide or a polypeptide having a Borrelia sequence. Other embodiments, may include at least a second, third, fourth, and so on, polynucleotide or polypeptide, wherein a first polynucleotide or polypeptide and a second or subsequent polynucleotide or polypeptide have different sequences. In more specific embodiments, the first polynucleotide may have a sequence of SEQ ID NO:1, SEQ ID NO:3, SEQ ID NO:5, SEQ ID NO:7, SEQ ID NO:9, SEQ ID NO:1, SEQ BD NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:101, SEQ ID NO:103, SEQ ID NO:105, SEQ ID NO:107, SEQ ID NO:109, SEQ ID NO:111, SEQ ID NO:113, SEQ ID NO:115, SEQ ID NO:116, SEQ ID NO:118, SEQ ID NO:120, SEQ ID NO:122, SEQ ID NO:124, SEQ ID NO:126, SEQ ID NO:128, SEQ ID NO:130, SEQ ID NO:134, SEQ ID NO:136, or SEQ ID NO:138, a complement, or fragment thereof and/or encode a polypeptide sequence as set forth in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ BD NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, SEQ ID NO:112, SEQ ID NO:114, SEQ ID SEQ ID NO:117, SEQ ID NO:119, SEQ ID NO:121, SEQ ID NO:123, SEQ ID NO:125, SEQ ID NO:127, SEQ ID NO:129, SEQ ID NO:131, SEQ ID NO:133, SEQ ID NO:135, SEQ ID NO:137, or SEQ ID NO:139, fragments, variants, or mimetics thereof, or closely related sequences. In other embodiments, antigenic fragments may be presented in a multi-epitope format, wherein a plurality of one or more antigenic fragments are engineered into a single molecule.

In certain embodiments of the invention a vaccine may comprise: (a) a pharmaceutically acceptable carrier (b) at least one polypeptide and/or polynucleotide encoding a polypeptide having a Borrelia sequence, including a fragment, variant or mimetic thereof, (c) at least one polypeptide and/or polynucleotide encoding a polypeptide that acts as an adjuvant or immunomodulator of antigen-specific immune response(s).

In various embodiments, the invention relates to methods of isolating Borrelia antigens and nucleic acids encoding such, as well as methods of using such isolated antigens for producing an immune response in a subject. Antigens of the invention may be used in vaccination of a subject against a Borrelia infection or disease.

Embodiments of the invention may include methods of immunizing an animal comprising providing to the animal at least one Borrelia antigen or antigenic fragment thereof, in an amount effective to induce an immune response. A Borrelia antigen can be derived from B burgdorferi or any other Borrelia species or sub-species. As discussed above, and described in detail below, the Borrelia antigens useful in the invention need not be native antigens. Rather, these antigens may have sequences that have been modified in any number of ways known to those of skill in the art, so long as they result in or aid in an antigenic or immune response.

In various embodiments of the invention, an animal or subject is a mammal. In some cases a mammal may be a mouse, horse, cow, pig, dog, or human. Alternatively, a subject may be selected from Deer, chickens, turtles, lizards, fish and other animals susceptible to Borrelia infection. In preferred embodiments, an animal or subject is a human.

Alternatively, these methods may be practiced in order to induce an immune response against a Borrelia species other than B. burgdorferi such as B. hernsii, B. garinii, and B. afzelii.

As used herein in the specification, “a” or “an” may mean one or more. As used herein, when used in conjunction with the word “comprising”, the words “a” or “an” may mean one or more than one. As used herein “another” may mean at least a second or more.

As used herein, “plurality” means more than one. In certain specific aspects, a plurality may mean 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or more, and any integer derivable therein, and any range derivable therein.

As used herein, “any integer derivable therein” means a integer between the numbers described in the specification, and “any range derivable therein” means any range selected from such numbers or integers.

As used herein, a “fragment” refers to a sequence having or having at least 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, or more contiguous residues of the recited SEQ ID NOs, but less than the full-length of the SEQ ID NOs. It is contemplated that the definition of “fragment” can be applied to amino acid and nucleic acid fragments.

As used herein, an “antigenic fragment” refers to a fragment, as defined above, that can elicit an immune response in an animal.

Reference to a sequence in an organism, such as a “Borrelia sequence” refers to a segment of contiguous residues that is unique to that genus, species, or sub-species of organism(s) or that constitutes a fragment (or full-length region(s)) found in that organism(s) (either amino acid or nucleic acid).

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Borrelia burgdorferi random expression library screen, round 1.

A complete random library comprised of plasmids that express Borrelia inserts as fusions with a secretory leader sequence was partitioned into 40 sublibraries. Each sublibrary contained between 1000 and 1500 library clones, which were co-delivered into mice with a plasmid expressing murine granulocyte-macrophage colony-stimulating factor (GMCSF). Following 2 boosts, mice were challenged with pathogen and protection from infection and disease were determined. The level of spirochete infection in the skin of an ear of each mouse was observed 17 days after challenge and scored on a 0 to 4 scale. The average density score and standard error for each group is tabulated in the lower panel. Measuring tibiotarsal joint diameters at weeks 3, 4, 5, and 6 post challenge was used to quantitate the severity of inflammatory disease. The increase in diameter over baseline was calculated by subtracting the average joint diameter of a group of uninfected mice from the measurement of each infected-mouse leg. The average change in swelling for mice in each group, at each time point, is plotted in the upper panel. Error bars display standard errors of the mean. The groups selected as positive for protection are indicated with an asterisk.

FIG. 2. RELI screen round 2 by matrix arraying.

The clones comprising the positively scoring sub-libraries in the first screening round were re-arrayed into new pools representing three dimensions of a cube: X (1 through 12), Y (1 through 16), and Z (1 through 18). These round 2 pools were tested for protective potential by genetic immunization without GMCSF co-delivery. Control groups included round 1 positives: #5, (A); #7, (B), #21, (C); #28, (E). Round 1 #22 was included as a retest (D). Two non-Borrelia expression libraries served as negative controls (F and G). Mice were challenged with spirochetes and scored by infection and disease readouts. Groups identified as protective by reduced spirochete densities are indicated with a plus sign. Those groups identified as protective by reduced inflammation are indicated with an asterisk.

FIG. 3. RELI Screen Round 3, Single Gene Fragment Testing.

Matrix and sequencing analyses of the round 2 results were used to identify single plasmids for testing in round 3. Increases in joint diameter were measured and inflammation was calculated as in previous rounds. The groups average increase in joint diameter at the 4 and 5-week time points are displayed. Control groups included: a pool of the clones carrying short ORFs (<50); a pool of clones carrying non-Borrelia inserts (not BBU); the pool of clones carrying the ORFs greater than 50 amino acids (>50); the same pool delivered only by gene gun (>50 gg); non-Borrelia library (Irrel Lib); non-immunized but challenged (NI). Data from mouse groups identified as protected at 85% confidence interval at either time point are displayed in black. Error bars show standard errors of the mean.

### DESCRIPTION OF ILLUSTRATIVE EMBODIMENTS

The present invention overcomes the current limitations of Borrelia vaccines by providing isolated nucleic acids and/or polypeptides from one or more members of the Borrelia genus that protect against disease. Certain embodiments include isolated nucleic acids and/or polypeptides from Borrelia burgdorferi. Compositions comprising isolated nucleic acids and polypeptides of a member of the Borrelia genus, as well as methods of using such compositions, may provide prophylactic or therapeutic immunization against members of the Borrelia genus. By introduction of one or more of the compositions of the present invention, a subject may be induced to produce antibodies against one or more bacteria of the Borrelia genus, specifically Borrelia burgdorferi.

Widespread human infection by members of the Borrelia genus represents a particular challenge for vaccinology. For example, Borrelia infections in humans may lead to Lyme disease or other disease conditions. Borreliosis is a multisystem illness with manifestations in the skin, heart, musculoskeletal, and central nervous systems. Thus, an effective treatment for Borrelia infections in humans and other vertebrate animals may be of clinical or prophylactic importance. Prophylactic methods may include reducing Borrelia infection in a population of deer or other common animal reservoirs of infection. In the present invention, the expression library immunization (ELI) process may be utilized to identify vaccine candidates against Borrelia infections and associated diseases. Clinically, some the goals of immunization against Borrelia infection may include reducing the severity of disease associated with primary infection or reducing the arthritis and other symptoms that can continue post-infection (PI).

The present invention provides compositions and methods for the immunization of vertebrate animals, including humans, against Borrelia infections. Compositions of the invention may comprise isolated nucleic acids encoding Borrelia polypeptide(s) and/or B. burgdorferi polypeptides, including complements, fragments, mimetics or closely related sequences, as antigenic components. Identification of the nucleic acids and polypeptides of the invention is typically carried out by using ELI methodology to screen Borrelia genome(s) (e.g., a B. burgdorferi sensu lato genome) for vaccine candidates. The compositions and methods of the invention may be useful for vaccination against Borrelia infections.

In various embodiments, a vaccine composition directed against a member of the Borrelia genus may be provided. The vaccine according to the present invention may comprise a Borrelia polynucleotide(s) and/or polypeptide(s). In particular embodiments, the Borrelia species is a B. burgdorferi spirochete bacterium. The vaccine compositions of the invention may provide protective or therapeutic capacities for a subject against Lyme disease and/or other Borreliosis-related conditions.

In still other embodiments, the invention may provide screening methods that include preparing and screening a cloned library via expression library immunization and identifying Borrelia genes that confer protection against or therapy for Borrelia infection. Additionally, methods may be used to identify and utilize polynucleotides and polypeptides derived from other related organism or by synthesizing a molecule that mimics the polypeptides of identified Borrelia polypeptides.

I. The Genus Borrelia

The genus Borrelia, of the family Spirochaetaceae, includes a variety of species and sub-species. In the genus Borrelia, approximately 20 species are associated with relapsing fevers and are transmitted by soft ticks or by lice in the case of Borrelia recurrentis. The genus includes Borrelia anserina; Borrelia barbouri; Borrelia burgdorferi group, which includes Borrelia afzelii, Borrelia andersonii, Borrelia bissettii, Borrelia burgdorferi, Borrelia garinii, Borrelia japonica, Borrelia lusitaniae Borrelia tanukii, Borrelia turdi, Borrelia valaisiana, Borrelia sp. A14S, Borrelia sp. AI-1, Borrelia sp. BC-1, Borrelia sp. CA128, Borrelia sp. CA13, Borrelia sp. CA27, Borrelia sp. CA28, Borrelia sp. CA29, Borrelia sp. CA31, Borrelia sp. CA370, Borrelia sp. CA372, Borrelia sp. CA378, Borrelia sp. CA394, Borrelia sp. CA395, Borrelia sp. CA404, Borrelia sp. CA443, Borrelia sp. CA446, Borrelia sp. CA8, Borrelia sp. FD-1, Borrelia sp. HN6, Borrelia sp. HN7, Borrelia sp. HN8, Borrelia sp. HNM13, Borrelia sp. HNM14, Borrelia sp. HNM19, Borrelia sp. I-77, Borrelia sp. Ir-3519, Borrelia sp. LV5, Borrelia sp. MI-2, Borrelia sp. MI-5, Borrelia sp. MI-8, Borrelia sp. MI-9, Borrelia sp. MOD-1, Borrelia sp. MOD-5, Borrelia sp. MOK-3a, Borrelia sp. MOS-1b, Borrelia sp. NE49, Borrelia sp. NE581, Borrelia sp. SCGT-10, Borrelia sp. SCGT-8a, Borrelia sp. SCI-2, Borrelia sp. SCW-30h, Borrelia sp. SI-1, Borrelia sp. SI-10, Borrelia sp. SM-1 and Borrelia sp. TXW-1; Borrelia coriaceae; Borrelia crocidurae; Borrelia duttonii; Borrelia hermsii, Borrelia hispanica, Borrelia lonestari, Borrelia miyamotoi, Borrelia parkeri, Borrelia persica, Borrelia recurrentis, Borrelia sinica, Borrelia theileri, Borrelia turicatae, Borrelia sp., Borrelia sp. 10MT, Borrelia sp. 5145, Borrelia sp. 5MT, Borrelia sp. EFL-S0100110, Borrelia sp. KR1, Borrelia sp. KR3, Borrelia sp. LB-2001, Borrelia sp. OkME1, Borrelia sp. strain Spain, Borrelia sp. TA1, Borrelia sp. TM1, and Borrelia sp. TM2. The term Borrelia as used herein refers to the genus Borrelia and/or its individual members.

Borrelia is a spirochete. Spirochetes are a group of phylogenetically-distinct prokaryotes that have a unique mode of motility by means of axial filaments (endoflagella). Spirochetes are widespread in viscous environments and they are found in the intestinal tracts of animals and the oral cavity of humans. The spirochetes have a unique cell surface which accompanies their unique type of motility. The endoflagella are contained within the periplasmic space between a rigid peptidoglycan helix and a multi-layer, flexible outer membrane sheath. When the filaments rotate within this space, the spirochetes move in cork-screw fashion. This mode of motility in spirochetes is thought to be an adaptation to Viscous environments such as aquatic sediments and the intestinal tracts of animals. For pathogens, this allows the spirochetes to hide their flagella, which are normally antigenic, from the host immune defenses. Spirochetes consist of an outer coat, the endoflagella in the periplasm and the protoplasmic cylinder. The protoplasmic cylinder is a complex of cytoplasm, internal cell membrane and peptidoglycan.

Spirochetes are usually much longer than they are wide, and often their width is below the resolving power of the light microscope. For example, Borrelia may have a length of 20-30 μm but a width of only 0.2-0.3 μm. Hence, most spirochetes cannot be viewed using conventional light microscopy. Dark-field microscopy is typically used to view spirochetes. Dark field microscopy utilizes a special condenser which directs light toward an object at a angle, rather than from the bottom. As a result, particles or cells are seen as light objects against a dark background.

The spirochetes are not classified as either Gram-positive or Gram-negative. When Borrelia burgdorferi is Gram-stained, the cells stain a weak Gram-negative by default, as safranin is the last dye used. Borrelia, like most spirochetes, does have an outer membrane that contains an LPS-like substance, an inner membrane, and a periplasmic space which contains a layer of peptidoglycan. Therefore, it has a Gram-negative bacterial type cell wall, despite its staining characteristics.

Borrelia burgdorferi can be cultivated in vitro. However, the bacterium typically requires a very complex growth medium called Barbour-Stoenner-Kelly (BSK) medium. It contains over thirteen ingredients in a rabbit serum base. Borrelia burgdorferi has an optimal temperature for growth of 32° C., in a microaerobic environment. The generation time is generally in the range of about 10-12 hrs.

The spirochetes causing Lyme disease are typically divided into several catagories, three of which have been firmly established and are well accepted as Borrelia burgdorferi sensu stricto, Borrelia garinii, and Borrelia afzelii.

The term used to collectively describe all three catagories is Borrelia burgdorferi sensu lato. The differences in these catagories are revealed by restriction fragment length polymorphism, (RFLP), multi-locus enzyme electrophoresis (MLEE) and small subunit ribosomal RNA (ssuRNA) sequences. All United States isolates fall into the Borrelia burgdorferi sensu stricto category. Examples of all three of these catagories have been found in Europe and Asia, although Borrelia garinii, and Borrelia afzelii predominate there.

As an example of the genus, the Borrelia burgdorferi outer membrane is composed of various unique outer surface proteins (Osp) (Osp A through OspF). They are presumed to play a role in virulence. Osp A and Osp B are by far the most abundant outer surface proteins. The genes encoding these proteins are transcribed from a common promoter, and are located on a 49 kb linear plasmid. The chromosome of Borrelia burgdorferi is also linear and is almost 1100 kb in size.

Borrelia burgdorferi have recently been shown to possess a unique type of extra chromosomal DNA, linear plasmids, which range in length from 0.5 to 50 kb (Bergstrom et al., 1991). These plasmids contain the genes encoding the two major outer surface proteins (Osp) expressed by B. burgdorferi, OspA and Osp B (Bergstrom et al., 1991).

Borrelia burgdorferi invades the blood and tissues of various infected mammals and birds. The natural reservoir for Borrelia burgdorferi is thought to be the white-footed mouse. Ticks transfer the spirochetes to the white-tailed deer, humans, and other warm-blooded animals after a blood meal on an infected animal. In humans, dogs, and many other animals, infection with Borrelia burgdorferi results in the pathology of Lyme Disease.

In various embodiments, other B. burgdorferi plasmids that are similar to those identified herein are contemplated as being used in the invention as described. Because several of the B. burgdorferi plasmids are highly homologous to each other, some protective clones, as described herein, have very close homologs in other B. burgdorferi plasmids. In particular, clone 1 on plasmid lp56 has between 95-97% identity to genes on plasmids cp32-6(AE001578), cp32-8 (AE001580), cp32-8 (AE001575), cp32-9 (AE001581), cp32-4(AE001577), cp32-3 (AE001576), cp32-7 (AE001579), clone 4 on plasmid lp25 has 91-93% identity to lp28-3 (AE000784) and lp36 (AE000788), clone 5 on cp32-7 has 89-100% identity to cp32-6 (AE001578), cp32-8 (AE001580), cp32-3 (AE001576), cp32-1 (AE001575), cp32-4 (AE001577), cp32-9 (AE001581), lp56 (AE001584), clone 6 on plasmid lp28-1 has 99% identity to lp36 (AE000788), clone 10 on plasmid cp32-7 has 84-95% identity to cp32-4 (AE001577), cp32-9 (AE001581), cpl8-2 (AF169008), cp32-8 (AE001580), cp32-1 (AE001575), cp32-3 (AE001576), cp32-6(AE001578), clone 11 on plasmid lp38 has 83% identity tolp28-3 (AE000784), clone 16 on cp32-6 has 93-99% identity to cp32-8 (AE001580), cp32-3 (AE001576), cp32-1 (AE001575), cp32-4 (AE001577), cp32-7 (AE001579), cp32-9 (AE001581), lp56 (AE001584), clone 18 on plasmid lp28-1 has 95-99% identity on lp36 (AE000788), lp28-3 (AE000784), lp56 (AE001584), lp17 (AE000793), lp16 (U43414), clone 19 on cp32-6 has 92-99% identity to cp32-7(AE001579), lp56(AE001584), cp32-3(AE001576), cp32-8 (AE001580), cp32-1 (AE001575), cp32-4(AE001577), cp32-9 (AE001581), clone 20 on plasmid cp32-3 has 98% identity to lp56 (AE001584), and clone 32 on plasmid lp5 has 85-88% identity to lp21 (AE001582), lp28-4 (AE000789), and lp25 (AE000785).

For example Clone #1 has the following identities or similarities as determined by the BLAST program accessible through the National Center for Biotechnology Information website. GenBank Accession number are provided in the first set parenthesis for each entry and are each incorporated herein by reference. (AE001584) Borrelia burgdorferi plasmid lp56, Identities=819/819 (100%); (AE001578) Borrelia burgdorferi plasmid cp32-6, Identities=797/819 (97%), Gaps=2/819 (0%); (AE001580) Borrelia burgdorferi plasmid cp32-8, Identities=794/819 (96%), Gaps=2/819 (0%); (AE001575) Borrelia burgdorferi plasmid cp32-1, Identities=794/819 (96%), Gaps=2/819 (0%); (AE001581) Borrelia burgdorferi plasmid cp32-9, Identities=796/822 (96%), Gaps=5/822 (0%); (AE001577) Borrelia burgdorferi plasmid cp32-4, Identities=793/819 (96%), Gaps=2/819 (0%); (AE001576) Borrelia burgdorferi plasmid cp32-3, Identities=789/819 (96%), Gaps=2/819 (0%); and (AE001579) Borrelia burgdorferi plasmid cp32-7, Identities=780/819 (95%), Gaps=2/819 (0%) In another example Clone #4 has the following identities or similarities as determined by the BLAST program accessible through the National Center for Biotechnology Information website. GenBank Accession number are provided in the first set parenthesis for each entry and are each incorporated herein by reference. (AE000785) Borrelia burgdorferi plasmid lp25, Identities=522/522 (100%); (AE000784) Borrelia burgdorferi plasmid lp28-3, Identities=488/522 (93%); and (AE000788) Borrelia burgdorferi plasmid lp36, Identities=467/510 (91%), Gaps=2/510 (0%)

In still other examples, Clone #5 has the following identities or similarities as determined by the BLAST program accessible through the National Center for Biotechnology Information website. GenBank Accession number are provided in the first set parenthesis for each entry and are each incorporated herein by reference. (AE001579) Borrelia burgdorferi plasmid cp32-7, Identities=197/197 (100%); (AE001578) Borrelia burgdorferi plasmid cp32-6, Identities=197/197 (100%); (AE001580) Borrelia burgdorferi plasmid cp32-8, Identities=193/197 (97%); (AE001576) Borrelia burgdorferi plasmid cp32-3, Identities=193/197 (97%); (AE001575) Borrelia burgdorferi plasmid cp32-1, Identities=193/197 (97%); (AE001577) Borrelia burgdorferi plasmid cp32-4, Identities=193/197 (97%); (AE001581) Borrelia burgdorferi plasmid cp32-9, Identities=190/197 (96%); and (AE001584) Borrelia burgdorferi plasmid lp56, Identities=177/197 (89%).

In yet further examples, Clone #6 has the following identities or similarities as determined by the BLAST program accessible through the National Center for Biotechnology Information website. GenBank Accession number are provided in the first set parenthesis for each entry and are each incorporated herein by reference. (AE000794) Borrelia burgdorferi plasmid lp28-1, Identities=860/860 (100%); and (AE000788) Borrelia burgdorferi plasmid lp36, Identities=691/693 (99%).

In still further examples, Clone #10 has the following identities or similarities as determined by the BLAST program accessible through the National Center for Biotechnology Information website. GenBank Accession number are provided in the first set parenthesis for each entry and are each incorporated herein by reference. (AE001579) Borrelia burgdorferi plasmid cp32-7, Identities=644/644 (100%); (AE001577) Borrelia burgdorferi plasmid cp32-4, Identities=283/297 (95%), Gaps=1/297 (0%); (AE001581) Borrelia burgdorferi plasmid cp32-9, Identities=278/294 (94%), Gaps=2/294 (0%); (AF169008) Borrelia burgdorferi circular plasmid cpl8-2, Identities=246/257 (95%); (AE001580) Borrelia burgdorferi plasmid cp32-8, Identities=248/261 (95%); (AE001575) Borrelia burgdorferi plasmid cp32-1, Identities=248/261 (95%); (AE001 576) Borrelia burgdorferi plasmid cp32-3, Identities=200/225 (88%), Gaps=6/225 (2%); and (AE00 1578) Borrelia burgdorferi plasmid cp32-6, Identities=198/235 (84%), Gaps=6/235 (2%)

In another example, Clone #11 has the following identities or similarities as determined by the BLAST program accessible through the National Center for Biotechnology Information website. GenBank Accession number are provided in the first set parenthesis for each entry and are each incorporated herein by reference. (AE000787) Borrelia burgdorferi plasmid lp38, Identities=127/127 (100%); and (AE000784) Borrelia burgdorferi plasmid lp28-3, Identities=106/127 (83%), Gaps=4/127 (3%).

In still another example, Clone #16 has the following identities or similarities as determined by the BLAST program accessible through the National Center for Biotechnology Information website. GenBank Accession number are provided in the first set parenthesis for each entry and are each incorporated herein by reference. (AE001578) Borrelia burgdorferi plasmid cp32-6, Identities=663/663 (100%); (AE001580) Borrelia burgdorferi plasmid cp32-8, Identities=658/663 (99%); (AE001576) Borrelia burgdorferi plasmid cp32-3, Identities=658/663 (99%); (AE001575) Borrelia burgdorferi plasmid cp32-1, Identities=658/663 (99%); (AE001577) Borrelia burgdorferi plasmid cp32-4, Identities=653/663 (98%); (AE001579) Borrelia burgdorferi plasmid cp32-7, Identities=648/663 (97%); (AE001581) Borrelia burgdorferi plasmid cp32-9, Identities=643/664 (96%), Gaps=1/664 (0%); and (AE001584) Borrelia burgdorferi plasmid lp56, Identities=620/663 (93%), Gaps=1/663 (0%).

In yet a further example, Clone #18 has the following identities or similarities as determined by the BLAST program accessible through the National Center for Biotechnology Information website. GenBank Accession number are provided in the first set parenthesis for each entry and are each incorporated herein by reference. (AE000794) Borrelia burgdorferi plasmid lp28-1, Identities=983/983 (100%); (AE000788) Borrelia burgdorferi plasmid lp36, Identities=506/509 (99%), Gaps=2/509 (0%); (AE000784) Borrelia burgdorferi plasmid lp28-3, Identities=452/470 (96%), Gaps=1/470 (0%); (AE001584) Borrelia burgdorferi plasmid lp56, Identities=451/470 (95%), Gaps=3/470 (0%); (AE000793) Borrelia burgdorferi plasmid lp17, Identities=451/470 (95%), Gaps=3/470 (0%); and (U43414) Borrelia burgdorferi linear plasmid lp16 DNA, Identities=451/470 (95%), Gaps=3/470 (0%).

In still another example, Clone #19 has the following identities or similarities as determined by the BLAST program accessible through the National Center for Biotechnology Information website. GenBank Accession number are provided in the first set parenthesis for each entry and are each incorporated herein by reference. (AE001578) Borrelia burgdorferi plasmid cp32-6, Identities=964/964 (100%); (AE001579) Borrelia burgdorferi plasmid cp32-7, Identities=962/964 (99%); (AE001584) Borrelia burgdorferi plasmid lp56, Identities=888/915 (97%); (AE001576) Borrelia burgdorferi plasmid cp32-3, Identities=905/964 (93%), Gaps=3/964 (0%); (AE001580) Borrelia burgdorferi plasmid cp32-8, Identities=904/964 (93%), Gaps=3/964 (0%); (AE001575) Borrelia burgdorferi plasmid cp32-1, Identities=904/964 (93%), Gaps=3/964 (0%); (AE001577) Borrelia burgdorferi plasmid cp32-4, Identities=898/964 (93%), Gaps=3/964 (0%); and (AE001581) Borrelia burgdorferi plasmid cp32-9, Identities=896/964 (92%), Gaps=3/964 (0%).

In yet a further example, Clone #20 has the following identities or similarities as determined by the BLAST program accessible through the National Center for Biotechnology Information website. GenBank Accession number are provided in the first set parenthesis for each entry and are each incorporated herein by reference. (AE001576) Borrelia burgdorferi plasmid cp32-3, Identities=278/278 (100%); and (AE001584) Borrelia burgdorferi plasmid lp56, Identities=252/255 (98%).

In another example, Clone #32 has the following identities or similarities as determined by the BLAST program accessible through the National Center for Biotechnology Information website. GenBank Accession number are provided in the first set parenthesis for each entry and are each incorporated herein by reference. (AE001583) Borrelia burgdorferi plasmid lp5, Identities=130/130 (100%); (AE001582) Borrelia burgdorferi plasmid lp21, Identities=115/130 (88%), Gaps=9/130 (6%); (AE000789) Borrelia burgdorferi plasmid lp28-4, Identities=115/130 (88%), Gaps=9/130 (6%); and (AE000785) Borrelia burgdorferi plasmid lp25, Identities=104/122 (85%).

II. Vaccines

The concept of vaccination/immunization is based on two fundamental characteristics of the immune system, namely specificity and memory of immune system components. Vaccination/immunization will initiate a response specifically directed to the antigen with which a subject was challenged. Furthermore, a population of memory B and T lymphocytes may be induced. Upon re-exposure to the antigen(s) or the pathogen an antigen(s) was derived from, the immune system will be primed to respond much faster and much more vigorously, thus endowing the vaccinated/immunized subject with immunological protection against a pathogen or disease state. This protection may also be augmented by administration of the same or different antigen repeatedly to subject of vaccination, termed a boost(s).

Vaccination is the artificial induction of actively-acquired immunity by administration of all or part of a non-pathogenic form or a mimetic of a disease-causing agent. The aim is to prevent a disease or treat a symptom of a disease, so the procedure may also be referred to as prophylactic or therapeutic immunization, respectively. In addition to actively-acquired immunity, passive immunization methods may also be used to provide a therapeutic benefit to a subject, see below.

In particular, genetic vaccination, also known as DNA immunization, involves administering an antigen-encoding expression vector(s) in vivo, in vitro or ex-vivo to induce the production of a correctly folded antigen(s) within an appropriate cell(s) or a target cell(s). The introduction of the genetic vaccine will cause an antigen to be expressed within those cells, an antigen typically being a pathogen-derived protein or proteins. The processed proteins will typically be displayed on the cellular surface of the transfected cells in conjunction with the Major Histocompatibility Complex (MHC) antigens of the normal cell. The display of these antigenic determinants in association with the MHC antigens is intended to elicit the proliferation of cytotoxic T-lymphocyte clones specific to the determinants. Furthermore, the proteins released by the expressing transfected cells can also be picked up, internalized or expressed by antigen-presenting cells to trigger a systemic humoral antibody responses.

A vaccine is a composition including an antigen derived from all or part of a pathogenic agent, or a mimetic thereof, that is modified to make it non-pathogenic and suitable for use in vaccination. The term vaccine is derived from Jenner's original vaccine that used cowpoxvirus isolated from calves to immunize a human against smallpox. Vaccines may include polynucleotides, polypeptides, attenuated pathogens, killed (or inactivated) pathogens, inactivated toxins, mimetics of an antigen and/or other antigenic materials that induce an immune response in a subject. The antigen(s) may be presented in various ways to the subject being immunized or treated. Types of vaccines include, but are not limited to genetic vaccines, virosomes, attenuated or inactivated whole organism vaccines, recombinant protein vaccines, conjugate vaccines, transgenic plant vaccines, toxoid vaccines, purified sub-unit vaccines, multiple genetically-engineered vaccines, anti-idiotype vaccines and other vaccine types known in the art.

An immune response may be an active or a passive immune response. Active immunity develops when the body is exposed to various antigens. It typically involves B lymphocytes and T lymphocytes, as described above. B lymphocytes (also called B cells) produce antibodies. Antibodies attach to a specific antigen and make it easier for phagocytes to destroy the antigen. Typically, T lymphocytes (T cells) attack antigens directly and may provide some control over the immune response. B cells and T cells develop that are specific for a particular antigen or antigen type. Passive immunization generally refers to the administration to a subject of antibodies or other affinity binding agents that are reactive with an antigen(s). One of the various goals of immunization is to provide a certain protection against or treatment for an exposure, an infection or a disease associated with the presence of a pathogen or an infection.

In certain cases, an immune response may be a result of adoptive immunotherapy. In adoptive immunotherapy lymphocyte(s) are obtained from a subject and are exposed or pulsed with an antigenic composition. The antigenic composition may comprise additional immunostimulatory agents or a nucleic acid encoding such agents, as well as adjuvants or excipients as described below. In certain instances, lymphocyte(s) may be obtained from the blood or other tissues of a subject. Lymphocyte(s) may be peripheral blood lymphocyte(s) and may be administered to the same or different subjects, referred to as autologous or heterologous donors respectively (for exemplary methods or compositions see U.S. Pat. Nos. 5,614,610, 5,766,588, 5,776,451, 5,814,295, 6,004,807 and 6,210,963).

The present invention includes methods of immunizing, treating or vaccinating a subject by contacting the subject with an antigenic composition comprising a Borrelia antigen. An antigenic composition may comprise a nucleic acid; a polypeptide; an attenuated pathogen, such as a virus, a bacterium, a fungus, or a parasite, which may or may not express a Borrelia antigen; a prokaryotic cell expressing a Borrelia antigen; a eukaryotic cell expressing a Borrelia antigen; a virosome and the like or a combination thereof. As used herein, an “antigenic composition” will typically comprise an antigen in a pharmaceutically acceptable formulation.

Antigen refers to any substance or molecule encoding a substance that an organism regards as foreign and therefore elicits an immune response, particularly in the form of specific antibodies or cell types reactive to the antigen. An antigenic composition may further comprise an adjuvant, an immunomodulator, a vaccine vehicle, and/or other excipients, as described herein and is known in the art (for example see Remington's Pharmaceutical Sciences). A Borrelia antigen is an antigen that is derived from any bacterium that is a member of the Borrelia genus. In particular embodiments a Borrelia antigen may be an antigen derived from B. borgderferi or B. afzelii.

Various methods of introducing an antigen or an antigen composition to a subject are known in the art. Vaccination methods include, but are not limited to DNA vaccination or genetic immunization (for examples see U.S. Pat. Nos. 5,589,466, 5,593,972, 6,248,565, 6,339,086, 6,348,449, 6,348,450, 6,359,054, each of which is incorporated herein by reference), edible transgenic plant vaccines (for examples see U.S. Pat. Nos. 5,484,719, 5,612,487, 5,914,123, 6,034,298, 6,136,320, and 6,194,560, each of which is incorporated herein by reference), transcutaneous immunization (Glenn et al., 1999 and U.S. Pat. No. 5,980,898, each of which is incorporated herein by reference), nasal or mucosal immunization (for examples see U.S. Pat. Nos. 4,512,972, 5,429,599, 5,707,644, 5,942,242, each of which is incorporated herein by reference); virosomes (Huang et al., 1979; Hosaka et al., 1983; Kaneda, 2000; U.S. Pat. Nos. 4,148,876; 4,406,885; 4,826,687; 5,565,203; 5,910,306; 5,985,318; each of which is incorporated herein by reference),687; U.S. Pat. Nos. 5,565,203; 5,910,306; 5,985,318, each of which is incorporated herein by reference), live vector and the like. Antigen delivery methods may also be combined with one vaccination regime.

Vaccines comprising an antigenic polypeptide or polynucleotide encoding a Borrelia polypeptide may present an antigen in a variety of contexts for the stimulation of an immune response. Some of the various vaccine contexts include attenuated pathogens, inactivated pathogens, toxoids, conjugates, recombinant vectors, and the like. Many of these vaccines may contain a mixture of different antigens derived from the same or different pathogens. Polypeptides of the invention may be mixed with, expressed by or couple to various vaccine components. Various vaccine compositions may provide an antigen directly or deliver an antigen producing composition, e.g., an expression construct, to a cell that subsequently produces or expresses an antigen or antigen-encoding molecule.

A. Genetic Vaccines

Immunization against an antigen or a pathogen may be carried out by inoculating, transfecting, or transducing a cell, a tissue, an organ, or a subject with a nucleic acid encoding an antigen. One or more cells of a subject may then express the antigen encoded by the nucleic acid. Thus, the antigen encoding nucleic acids may comprise a “genetic vaccine” useful for vaccination and immunization of a subject. Expression in vivo of the nucleic acid may be, for example, from a plasmid type vector, a viral vector, a viral/plasmid construct vector, or an LEE or CEE construct.

In preferred aspects, the nucleic acid comprises a coding region that encodes all or part of an antigenic peptide, or an immunologically functional equivalent thereof. Of course, the nucleic acid may comprise and/or encode additional sequences, including but not limited to those comprising one or more immunomodulators or adjuvants. A nucleic acid may be expressed in an in vivo, in vitro or ex vivo context, and in certain embodiments the nucleic acid comprises a vector for in vivo replication and/or expression. For exemplary compositions and methods see U.S. Pat. Nos. 5,589,466, 6,200,959, 6,339,068, and the like.

B. Polypeptide Vaccines

In accordance with the present invention, one may utilize antigen compositions containing one or more Borrelia polypeptides, as well as variants or mimics thereof, to induce an immune response in a subject. Borrelia polypeptides of the invention may be synthesized or purified from a natural or recombinant source and used as a component of a polypeptide vaccine. In various embodiments, polypeptides may include fusion proteins, isolated polypeptides, polypeptides conjugated with other immunogenic molecules or substances, polypeptide mixtures with other immunogenic molecules or substances, and the like (for exemplary compositions and methods see U.S. Pat. Nos. 5,976,544, 5,747,526, 5,725,863, and 5,578,453).

C. Purified Sub-Unit Vaccines

Compositions and methods described herein may be used to isolate a portion of a pathogen for use as a sub-unit vaccine. Sub-unit vaccines may utilize a partially or substantially purified molecule of a pathogen as an antigen. Polynucleotides and/or polypeptides of the invention may serve as a sub-unit vaccine or be used in combination with or be included in a sub-unit vaccine for Borrelia Methods of sub-unit vaccine preparation may include the extraction of certain antigenic molecules from a bacterium of the Borrelia genus, and/or other pathogens by known purification methods. The preparation of a sub-unit vaccine may neutralize the pathogenicity of an entire pathogen rendering the vaccine non-infectious. Examples include influenza vaccine (viral surface hemagglutinin molecule) and Haemophilus meningitis vaccine (capsular polysaccharide molecule). Protein sub-units may be produced in non-pathogenic microbes by genetic engineering techniques making production much safer.

D. Conjugate Vaccines

The compositions and antigens of the invention may be conjugated to other molecules to produce a conjugate vaccine. Polysaccharides found to be poorly immunogenic by themselves have been shown to be quite good immunogens once they are conjugated to an immunogenic protein (U.S. Pat. No. 4,695,624, incorporated herein by reference) Conjugate vaccines may also be used to enhance the immunogenicity of an antigenic polypeptide. Conjugate vaccines utilize the immunologic properties of certain peptides to enhance the immunologic properties of glycolipids, polysaccharides, other polypeptides and the like. Certain embodiments of the invention contemplate using conjugates to enhance the immunogenicity of the polynucleotides and polypeptides of the invention. Examples of conjugate vaccines can be found in U.S. Pat. Nos. 6,309,646, 6,299,881, 6,248,334, 6,207,157, 5,623,057; each of which is incorporated herein by reference.

E. Virus-like Particle (VLP) Vaccines

Polynucleotides and polypeptides of the invention may be used in conjunction with VLP vaccines. In many virus species, virus proteins are capable of assembling in the absence of nucleic acid to form so-called virus-like particles or VLPs. Similarly, the proteins which normally cooperate together with nucleic acid to form the virus core can assemble in the absence of nucleic acid to form so-called core-like particles (CLPs). The terms “virus-like particles” and “core-like particles” will be used to designate assemblages of virus proteins (or modified or chimeric virus proteins) in the absence of a viral genome. The addition of antigenic peptide in the context of these particles may be especially useful in the development of vaccines for oral or other mucosal routes of administration (for examples see U.S. Pat. No. 5,667,782, which is hereby incorporated by reference). In other embodiments of the invention virosome may also be used. Examples of virosome compositions and methodology can be found in U.S. Pat. Nos. 4,148,876, 4,406,885, 4,826,687, and Kaneda, 2000, each of which is incorporated herein by reference.

F. Cell Mediated Vaccines

An alternative method of presenting antigens is to use genetically modified cells as an expression or delivery vehicle for polynucleotides or polypeptides of the invention. For example, cells may be isolated from a subject or another donor and transformed with a genetic construct that expresses an antigen, as described herein. Following selection, antigen-expressing cells are cultured as needed. The cells may then be introduced or reintroduced to a subject, where these cells express an antigen and induce an immune response (see U.S. Pat. Nos. 6,228,640, 5,976,546 5,891,432 and the like).

In certain embodiments, cell mediated vaccines may include vaccines comprising antigen presenting cells (APC). A cell that displays or presents an antigen normally or preferentially with a class II major histocompatibility molecule or complex to an immune cell is an “antigen presenting cell.” Secreted or soluble molecules, such as for example, cytokines and adjuvants, may also aid or enhance the immune response against an antigen. Such molecules are well known to one of skill in the art, and various examples are described herein.

The dendritic cell (DC) is a cell type that may be used for cell-mediated vaccination, as they are potent antigen presenting cells, effective in the stimulation of both primary and secondary immune responses (Steinman, 1999; Celluzzi and Falo, 1997). It is contemplated in the present invention that the exposure or transformation of dendritic cells to an antigenic composition of the invention, will typically elicit a potent immune response specific for a bacterium of the Borrelia genus.

G. Edible Vaccines

An edible vaccine is a plant, food plant or food stuff that is used in delivering an antigen that is protective against an infectious disease, a pathogen, an organism, a bacteria, a virus or an autoimmune disease. In particular, the invention provides for an edible vaccine that induces a state of immunization against a member of the Borrelia genus. The present invention may also include gene constructs or chimeric gene constructs comprising a coding sequence of at least one of the polypeptides, peptides, or fragments thereof of the invention, plant cells and transgenic plants transformed with said gene constructs or chimeric gene constructs, and methods of preparing an edible vaccine from these plant cells and transgenic plants. For exemplary methods see U.S. Patent publication 20020055618 and U.S. Pat. Nos. 5,914,123; 6,034,298; 6,136,320; 6,444,805; and 6,395,964, which are incorporated herein by reference. The present invention also provides methods of treating disease or infection with edible vaccines and compositions comprising edible vaccines according to the invention.

Numerous plants may be useful for the production of an edible vaccine, including: tobacco, tomato, potato, eggplant, pepino, yam, soybean, pea, sugar beet, lettuce, bell pepper, celery, carrot, asparagus, onion, grapevine, muskmelon, strawberry, rice, sunflower, rapeseed/canola, wheat, oats, maize, cotton, walnut, spruce/conifer, poplar and apple. An edible vaccine may include a plant cell transformed with a nucleic acid construct comprising a promoter and a sequence encoding a peptide of the invention. The sequence may optionally encode a chimeric protein, comprising, for example, a cholera toxin subunit B peptide fused to the peptide. Plant promoters of the invention include, but are not limited to CaMV 35S, patatin, mas, and granule-bound starch synthase promoters. Additional useful promoters and enhancers are described in WO 99/54452, incorporated herein by reference.

The edible vaccine of the invention can be administered to a mammal suffering from or at risk of disease or infection. Preferably, an edible vaccine is administered orally, e.g., consuming a transgenic plant of the invention. The transgenic plant can be in the form of a plant part, extract, juice, liquid, powder, or tablet. The edible vaccine can also be administered via an intranasal route.

H. Live Vector Vaccines

In another embodiment, a live vector vaccine may be prepared comprising non-pathogenic micro-organisms, e.g., viruses or bacteria containing poynucleotides or nucleic acids encoding the peptides or antigens of the present invention cloned into the same or different micro-organisms. Live vector vaccines, also called “carrier vaccines” and “live antigen delivery systems”, comprise an exciting and versatile area of vaccinology (Levine et al., 1990; Morris et al., 1992; Barletta et al., 1990; Dougan et al., 1987; and Curtiss et al., 1989; U.S. Pat. Nos. 5,783,196; 5,648,081; and 6,413,768; each of which is incorporated herein by reference). In this approach, a live viral or bacterial vaccine is modified so that it expresses protective foreign antigens of another microorganism, and delivers those antigens to the immune system, thereby stimulating a protective immune response. Live bacterial vectors that are being promulgated include, among others, attenuated Salmonella (Levine et al., 1990; Morris et al., 1992; Dougan et al., 1987; and Curtiss et al., 1989), Bacille Calmette Guerin (Barletta et al., 1990), Yersinia enterocolitica (Van Damme et al., 1992), V. cholerae Ol (Viret et al., 1993)) and E. coli (Hale, 1990). The use of attenuated organisms as live vectors/vaccines expressing protective antigens of relevant pathogens is well-known in the field.

Attenuated Pathogen Vaccines

In certain embodiments, an antigen may be incorporated in or coupled to an attenuated pathogen, bacteria, virus or cell, which may encode, express, or is coupled to the antigen. Attenuation may be accomplished by genetic engineering, altering culture conditions, or physical treatment, such as chemical or heat inactivation. An antigen encoded by or present on or in an attenuated pathogen is one which when expressed or exposed is capable of inducing an immune response and providing protection and/or therapy in an animal against a bacterium or bacteria of the Borrelia genus from which one or more antigen(s) was derived, or from a related organism. Borrelia antigens may be introduced into an attenuated pathogen by way of DNA encoding the same. For exemplary methods and compositions see U.S. Pat. Nos. 5,922,326, 5,607,852 and 6,180,110.

Killed Pathogen Vaccines

A Borrelia antigen may also be associated with a killed or inactivated pathogen or cell. Killed pathogen vaccines include preparations of wild-type pathogens, or a closely-related pathogen, that has been treated to make them non-viable (inactivated). Methods of inactivation includes heat-killing of a pathogen. One advantage of heat killing is that it leaves no extraneous residue, but may alter protein conformations and hence immunogenic specificity, however it is useful for vaccines in which the immunogenic molecule is a polysaccharide. Alternative methods of killing include chemicals (β-propio-lacone or formaldehyde), which may leave a toxic residue, but does not alter protein conformations significantly and preserves immunogenic specificity. For exemplary methods and compositions see U.S. Pat. Nos. 6,303,130, 6,254,873, 6,129,920 and 5,523,088.

Humanized Antibodies

Polypeptides, fragments or mimetics thereof, of the invention may be used to produce anti-idiotypic antibodies for use in a Borrelia vaccine. In an anti-idiotype vaccine an antigen is an antibody against the Fab end of a second antibody which was raised against an antigenic molecule of a pathogen. The Fab end of the first antibody will have the same antigenic shape as the antigenic molecule of the pathogen and may then be used as an antigen (see exemplary U.S. Pat. Nos. 5,614,610, 5,766,588). “Humanized” antibodies for use herein may be antibodies from non-human species wherein one or more selected amino acids have been exchanged for amino acids more commonly observed in human antibodies. This can be readily achieved through the use of routine recombinant technology, particularly site-specific mutagenesis.

III. Antigen/Vaccine Screening Methods

Methods of screening for at least one test polypeptide or test polynucleotide encoding a polypeptide for an ability to produce an immune response may comprise (i) obtaining at least one test polypeptide or test polynucleotide by (a) modifying the amino acid sequence of a known antigenic polypeptide or polynucleotide sequence of a polynucleotide encoding a known antigenic polypeptide; (b) obtaining a homolog of a known antigenic sequence of a polynucleotide encoding such a homolog, or (c) obtaining a homolog of a known antigenic sequence or a polynucleotide encoding such a homolog and modifying the amino acid sequence of the homolog or the polynucleotide sequence of the polynucleotide encoding such a homolog; and (ii) testing the test polypeptide or test polynucleotide under appropriate conditions to determine whether the test polypeptide is antigenic or the test polynucleotide encodes an antigenic polypeptide.

A method of screening may include obtaining a test polypeptide by modifying the amino acid sequence or obtaining a homolog of a least one polypeptide of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:10, SEQ ID NO:112, SEQ ID NO:114, SEQ ID NO:117, SEQ ID NO:119, SEQ ID NO:121, SEQ ID NO:123, SEQ ID NO:125, SEQ ID NO:127, SEQ ID NO:129, SEQ ID NO:131, SEQ ID NO:133, SEQ ID NO:135, SEQ ID NO:137, SEQ ID NO:139 or fragment thereof. The method of screening may also include a test polypeptide comprising an amino acid sequence of at least one of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:10, SEQ ID NO:112, SEQ ID NO:114, SEQ ID NO:117, SEQ ID NO:119, SEQ ID NO:121, SEQ ID NO:123, SEQ ID NO:125, SEQ ID NO:127, SEQ ID NO:129, SEQ ID NO:131, SEQ ID NO:133, SEQ ID NO:135, SEQ ID NO:137, SEQ ID NO:139 or fragment thereof, which has been modified.

In other embodiments the method of screening may also include obtaining a test polynucleotide comprising a polynucleotide encoding a modified amino acid sequence of or a homolog of at least one polypeptide having a sequence of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, SEQ ID NO:112, SEQ ID NO:114, SEQ ID NO:117, SEQ ID NO:119, SEQ ID NO:121, SEQ ID NO:123, SEQ ID NO:125, SEQ ID NO:127, SEQ ID NO:129, SEQ ID NO:131, SEQ ID NO:133, SEQ ID NO:135, SEQ ID NO:137, SEQ ID NO:139 or fragment thereof or obtaining a test polynucleotide comprising modifying the polynucleotide sequence of at least one of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:101, SEQ ID NO:103, SEQ ID NO:105, SEQ ID NO:107, SEQ ID NO:109, SEQ ID NO:111, SEQ ID NO:113, SEQ ID NO:115, SEQ ID NO:116, SEQ ID NO:118, SEQ ID NO:120, SEQ ID NO:122, SEQ ID NO:124, SEQ ID NO:126, SEQ ID NO:128, SEQ ID NO:130, SEQ ID NO:132, SEQ ID NO:134, SEQ ID NO:136, or SEQ ID NO:138, or fragment thereof. In various embodiments a method of screening may further comprise identifying at least one test polypeptide as being antigenic or at least one test polynucleotide as encoding an antigenic polypeptide.

The methods described may include placing an identified antigenic polypeptide or the polynucleotide encoding an antigenic polypeptide in a pharmaceutical composition. The methods may also include using an identified antigenic polypeptide or polynucleotide encoding an antigenic polypeptide to vaccinate a subject. In certain aspects a subject may be vaccinated against a member of the Borrelia genus and in particular B. burgdorferi. Additionally, the subject may be vaccinated against a non-B. burgdorferi disease.

Additional embodiments include a method of preparing a vaccine including obtaining an antigenic polypeptide or a polynucleotide encoding an antigenic polypeptide, as determined to be antigenic by known screening methods and/or screening methods described herein, and placing a polypeptide or a polynucleotide in a vaccine composition. A vaccine composition may be used in vaccinating a subject by preparing a vaccine as described and vaccinating a subject with the vaccine.

IV. Borrelia Antigens

Antigens of the invention are typically isolated from members of Borrelia genus, in particular B. burgdorferi or B. afzelii. In particular embodiments, the immunization of vertebrate animals according to the present invention includes a cloned library of Borrelia genomic and/or plasmid DNA in expression constructs. In various embodiments, a DNA expression construct may be in the context of a linear expression elements (“LEEs”) and/or circular expression elements (“CEEs”), which typically encompass a complete gene (promoter, coding sequence, and terminator). These LEEs and CEEs can be directly introduced into and expressed in cells or an intact organism to yield expression levels comparable to those from a standard supercoiled, replicative plasmid (Sykes and Johnston, 1999). In specific embodiments, a cloned expression library of Borrelia (e.g., B. burgdorferi or B. afzelii) is provided. Expression library immunization, ELI herein, is well known in the art (U.S. Pat. No. 5,703,057, specifically incorporated herein by reference). In certain embodiments, the invention provides an ELI method applicable to virtually any pathogen and requires no knowledge of the biological properties of the pathogen. The method operates on the assumption, generally accepted by those skilled in the art, that all the potential polypeptide determinants for any pathogen are encoded by its genome. The inventors have previously devised methods of identifying vaccines using a genomic expression library representing all of the antigenic determinants of a pathogen (U.S. Pat. No. 5,703,057). The method uses to its advantage the simplicity of genetic immunization to sort through a genome for immunological reagents in an unbiased, systematic fashion.

The preparation of an expression library is performed using the techniques and methods familiar one of skill in the art (Sambrook et al., 2001). The pathogen's genome, may or may not be known. Thus one obtains DNA (or cDNA), representing substantially the entire genome of the pathogen (e.g., B. burgdorferi). The DNA is broken up, by physical fragmentation or restriction endonuclease, into segments of some length so as to provide a library of about 105 members. The library is then tested by inoculating a subject with purified DNA of the library or sub-library, and the subject challenged with a pathogen, wherein immune protection of the subject from pathogen challenge indicates a clone that confers a protective immune response against infection.

In some embodiments of the invention, a Borrelia antigen may be obtained by methods comprising: (a) preparing a cloned expression library from fragmented nucleic acids (e.g., genomic or plasmid DNA) of a member of the Borrelia genus; (b) administering at least one clone of the library in a pharmaceutically acceptable carrier into an animal; and (c) expressing at least one Borrelia antigen in the animal. The expression library may comprise at least one or more polynucleotides having a sequence of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:101, SEQ ID NO:103, SEQ ID NO:105, SEQ ID NO:107, SEQ ID NO:109, SEQ ID NO:111, SEQ ID NO:113, SEQ ID NO:115, SEQ ID NO:116, SEQ ID NO:118, SEQ ID NO:120, SEQ ID NO:122, SEQ ID NO:124, SEQ ID NO:126, SEQ ID NO:128, SEQ ID NO:130, SEQ ID NO:132, SEQ ID NO:134, SEQ ID NO:136, or SEQ ID NO:138, a complement, a fragment, or a closely related sequences thereof. The polynucleotides of SEQ ID NO:1, SEQ ID NO:5, SEQ ID NO:9, SEQ ID NO:13, SEQ ID NO:17, SEQ ID NO:21, SEQ ID NO:25, SEQ ID NO:29, SEQ ID NO:33, SEQ ID NO:37, SEQ ID NO:41, SEQ ID NO:45, SEQ ID NO:49, SEQ ID NO:53, SEQ ID NO:57, SEQ ID NO:61, SEQ ID NO:65, SEQ ID NO:69, SEQ ID NO:73, SEQ ID NO:77, SEQ ID NO:81, SEQ ID NO:85, SEQ ID NO:89, SEQ ID NO:93, SEQ ID NO:97, SEQ ID NO:101, SEQ ID NO:105, SEQ ID NO:109, SEQ ID NO:118, SEQ ID NO:122, SEQ ID NO:124, SEQ ID NO:128, SEQ ID NO:132, or SEQ ID NO:136 represent exemplary gene fragments identified using ELI and related technology, as described herein. In addition, polynucleotides of SEQ ID NO:3, SEQ ID NO:7, SEQ ID NO:11, SEQ ID NO:15, SEQ ID NO:19, SEQ ID NO:23, SEQ ID NO:27, SEQ ID NO:31, SEQ ID NO:35, SEQ ID NO:39, SEQ ID NO:43, SEQ ID NO:47, SEQ ID NO:51, SEQ ID NO:55, SEQ ID NO:59, SEQ BD NO:63, SEQ ID NO:67, SEQ ID NO:71, SEQ ID NO:75, SEQ ID NO:79, SEQ ID NO:83, SEQ ID NO:87, SEQ ID NO:91, SEQ ID NO:95, SEQ ID NO:99, SEQ ID NO:103, SEQ ID NO:107, SEQ ID NO:111, SEQ ID NO:113, SEQ ID NO:115, SEQ ID NO:116, SEQ ID NO:120, SEQ ID NO:126, SEQ ID NO:130, SEQ ID NO:134, or SEQ ID NO:138 are representative of exemplary full length gene sequences or full length recombination cassettes identified using ELI and related technologies, as described herein. The expression library may be cloned in a genetic immunization vector or any other suitable expression construct. The construct may comprise a gene encoding a mouse ubiquitin polypeptide positioned such that it produces a Borrelia/mouse, ubiquitin/antigen fusion protein designed to link the expression library polynucleotides to the ubiquitin gene. The vector may comprise a promoter operable in eukaryotic cells, for example a CMV promoter, or any other suitable promoter. In such methods, the polynucleotide may be administered by an intramuscular injection intradermal, epidermal injection, or particle bombardment. The polynucleotide may likewise be administered by intravenous, subcutaneous, intralesional, intraperitoneal, oral, other mucosal, or inhaled routes of administration. In some specific, exemplary embodiments, the administration may be via intramuscular injection of at least 0.01 μg to 200 μg of the polynucleotide. In other exemplary embodiments, administration may be epidermal injection of at least 0.001 μg to 5.0 μg of the polynucleotide. In some cases, a second administration, for example, an intramuscular injection and/or epidermal injection/bombardment, may be administered at least about three weeks after the first administration. In these methods, the polynucleotide may be, but need not be, cloned into a viral expression vector, for example, a viral expression vector, including adenovirus, herpes-simplex virus, retrovirus or adeno-associated virus vectors. The polynucleotide may also be administered in any other method disclosed herein or known to those of skill in the art.

In still other embodiments, a Borrelia antigen(s) maybe obtained by methods comprising: (a) preparing a pharmaceutical composition comprising at least one polynucleotide encoding an Borrelia antigen or fragment thereof; (b) administering one or more clones of the library in a pharmaceutically acceptable carrier into an animal; and (c) expressing one or more Borrelia antigens in the animal. The one or more polynucleotides can be comprised in one or more expression vectors.

Alternatively, methods of obtaining Borrelia antigen(s) may comprise: (a) preparing a pharmaceutical composition of at least one Borrelia antigen or an antigenic fragment thereof; and (b) administering the at least one antigen or fragment into an animal. The antigen(s) may be administered by an intramuscular injection, intradermal injection, intravenous injection, parenteral injection, epidermal injection, inhalation, oral, or other mucosal routes.

Also described herein, are methods of obtaining polynucleotide sequences effective for generating an immune response against members of the Borrelia genus, in particular B. burgdorferi, in a non-human animal comprising: (a) preparing a cloned expression library from fragmented genomic DNA of a bacterium selected from the Borrelia genus; (b) administering one or more clones of the library in a pharmaceutically acceptable carrier into the animal in an amount effective to induce an immune response; and (c) selecting from the library the polynucleotide sequences that induce an immune response, wherein the immune response in the animal is protective against infection by one or more members of the genus Borrelia. Such methods may further comprise testing the animal for immune resistance against a Borrelia infection by challenging the animal with Borrelia. In some cases, the genomic or plasmid DNA has been fragmented physically or by restriction enzymes. DNA fragments may be, on average, about 200-1000 base pairs in length. In some cases, each clone in the library may comprise a gene encoding a mouse ubiquitin fusion polypeptide designed to link the expression library polynucleotides to the ubiquitin gene, but this is not required in all cases. In some cases, the library may comprise about 1×10² to about 1×10⁶ clones; in more specific cases, the library could have 1×10⁵ clones. In some preferred methods, about 0.0001 μg to about 200 μg of DNA, from the clones is administered into the animal. In some situations the genomic or plasmid DNA, gene or cDNA is introduced by intramuscular injection or epidermal injection or bombardment. In some versions of these protocols, the cloned expression library further comprises a promoter operably linked to the DNA that permits expression in a vertebrate animal cell.

The application also discloses methods of preparing antigens that confer protection against infection in a vertebrate animal comprising the steps of: (a) preparing a cloned expression library from fragmented genomic or plasmid DNA of bacterium of the genus Borrelia; (b) administering one or more clones of the library in a pharmaceutically acceptable carrier into the animal in an amount effective to induce an immune response; (c) selecting from the library the polynucleotide sequences that induce an immune response (d) expressing the polynucleotide sequences in cell culturesuch as a eukaryotic or prokaryotic expression system; and (e) purifying the polypeptide(s) expressed in the cell culture. Often, these methods further comprise testing the animal for immune resistance against infection by challenging the animal with one or more bacteria or other pathogens.

In yet other embodiments the invention relates to methods of preparing antibodies against a Borrelia antigen comprising the steps of: (a) identifying a Borrelia antigen that confers immune resistance against Borrelia infection when challenged with a selected member of the Borrelia genus, which may or may not be the bacterium from which the antigen was prepared; (b) generating an immune response in a vertebrate animal with the antigen identified in step (a); and (c) obtaining antibodies produced in the animal.

The invention also relates to methods of preparing antibodies against a Borrrelia polypeptide that is immunogenic, and not necessarily protective as a vaccine. For example Borrelia-specific antibodies might be useful in research analyses, diagnosis or antibody-therapy. Immunizing animals with the identified antigen might produce antibodies, or expressing the gene encoding the antibody could produce them. In other method of producing Borrelia antibodies, the identified antigen might be used for panning against a phage library. This procedure would isolate phage-antibodies in vitro.

A. Nucleic Acids

The present invention provides compositions comprising Borrelia polynucleotides and methods of using these compositions to induce a protective immune response in vertebrate animals. In certain embodiments, an animal may be challenged with a Borrelia infection.

In various embodiments of the invention, genes and polynucleotides encoding Borrelia polypeptides, as well as fragments thereof, are provided. In other embodiments, a polynucleotide encoding a Borrelia polypeptide or a polypeptide fragment may be expressed in prokaryotic or eukaryotic cells. The expressed polypeptides or polypeptide fragments may be purified for use as Borrelia antigens in the vaccination of vertebrate animals or in generating antibodies immunoreactive with Borrelia polypeptides or polypeptide fragments.

The present invention is not limited in scope to the genes of any particular bacterium of the Borrelia genus. One of ordinary skill in the art could, using the nucleic acids and compositions described herein, readily identify related bacterium or protein homologs in the Borrelia genus. In addition, it should be clear that the present invention is not limited to the specific nucleic acids disclosed herein. As discussed below, a specific “Borrelia” gene or polynucleotide fragment may contain a variety of different bases and yet still produce a corresponding polypeptide that is functionally indistinguishable, and in some cases structurally indistinguishable, from the polynucleotide sequences disclosed herein.

1. Nucleic Acids Encoding Borrelia Antigens

The present invention provides polynucleotides encoding antigenic Borrelia polypeptides capable of inducing a protective immune response in vertebrate animals and for use as an antigen to generate anti-Borrelia antibodies or antibodies reactive with other pathogens. In certain instances, it may be desirable to express Borrelia polynucleotides encoding a particular antigenic Borrelia polypeptide domain or sequence to be used as a vaccine or in generating anti-Borrelia antibodies or antibodies reactive with other pathogens. Nucleic acids according to the present invention may encode an entire Borrelia gene, or any other fragment of the Borrelia sequences set forth herein. The nucleic acid may be derived from genomic or plasmid DNA, i.e., cloned directly from the genome or plasmids of a particular organism. In other embodiments, however, the nucleic acid may comprise complementary DNA (cDNA) or synthetically built DNA. A protein may be derived from the designated sequences for use in a vaccine or in methods for isolating antibodies.

The term “cDNA” is intended to refer to DNA prepared using messenger RNA (mRNA) as a template. The advantage of using a cDNA, as opposed to DNA amplified or synthesized from a genomic or plasmid DNA template or a non-processed or partially processed RNA template, is that a cDNA primarily contains coding sequences comprising the open reading frame (ORF) of the corresponding protein. There may be times when the full or partial genomic sequence is preferred, such as where the non-coding regions are required for optimal expression.

In still further embodiments, a Borrelia polynucleotide from a given species may be represented by natural variants that have slightly different nucleic acid sequences but, nonetheless, encode the same polypeptide (see Table 1 below). In addition, it is contemplated that a given Borrelia polypeptide from a species may be generated using alternate codons that result in a different nucleic acid sequence but encodes the same polypeptide.

As used in this application, the term “a nucleic acid encoding a Borrelia polynucleotide” refers to a nucleic acid molecule that has been isolated free of total cellular nucleic acid. The term “functionally equivalent codon” is used herein to refer to codons that encode the same amino acid, such as the six codons for arginine or serine (Table 1, below), and also refers to codons that encode biologically equivalent amino acids, as discussed in the following pages.

Allowing for the degeneracy of the genetic code, sequences are considered essentially the same as those set forth in a Borrelia gene or polynucleotide that have at least about 50%, usually at least about 60%, more usually about 70%, most usually about 80%, preferably at least about 90% and most preferably about 95% of nucleotides that are identical to the nucleotides of a given Borrelia gene or polynucleotide. Sequences that are essentially the same as those set forth in a Borrelia gene or polynucleotide may also be functionally defined as sequences that are capable of hybridizing to a nucleic acid segment containing the complement of a Borrelia polynucleotide under standard conditions. The term closely related sequences refers to sequences with either substantial sequence similarity or sequence that encode proteins that perform or invoke similar antigenic responses as described herein. The term closely related sequence is used herein to designate a sequence with a minimum or 50% similarity with a polynucleotide or polypeptide with which it is being compared.

The DNA segments of the present invention include those encoding biologically functional equivalent Borrelia proteins and peptides, as described above. Such sequences may arise as a consequence of codon redundancy and amino acid functional equivalency that are known to occur naturally within nucleic acid sequences and the proteins thus encoded. Alternatively, functionally equivalent proteins or peptides may be created via the application of recombinant DNA technology, in which changes in the protein structure may be engineered, based on considerations of the properties of the amino acids being exchanged. Changes may be engineered through the application of site-directed mutagenesis techniques or may be introduced randomly and screened later for the desired function, as described below.

TABLE 1

| | |
|---|---|
| Amino Acids | Codons |

| | | | |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

2. Oligonucleotides

Naturally, the present invention also encompasses oligonucleotides that are complementary, or essentially complementary to the sequences of an Borrelia polynucleotide. Nucleic acid sequences that are “complementary” are those that are capable of base-pairing according to the standard Watson-Crick complementary rules. As used herein, the term “complementary sequences” means nucleic acid sequences that are substantially complementary, as may be assessed by the same nucleotide comparison set forth above, or as defined as being capable of hybridizing to the nucleic acid segment of an Borrelia polynucleotide under relatively stringent conditions such as those described herein.

Alternatively, the hybridizing segments may be shorter oligonucleotides. Sequences of 17 bases long should occur only once in the human genome and, therefore, suffice to specify a unique target sequence. Although shorter oligomers are easier to make and increase in vivo accessibility, numerous other factors are involved in determining the specificity of hybridization. Both binding affinity and sequence specificity of an oligonucleotide to its complementary target increases with increasing length. It is contemplated that exemplary oligonucleotides of 8, 9, 10, 11, 12, 13, 14, 15, 16, 17, 18, 19, 20, 25, 30, 35, 40, 45, 50, 55, 60, 65, 70, 75, 80, 85, 90, 95, 100 or more base pairs will be used, although others are contemplated. Longer polynucleotides encoding 250, 500, 1000, 1212, 1500, 2000, 2500, 3000 or 3500 bases and longer are contemplated as well. Such oligonucleotides or polynucleotides will typically find use, for example, as probes in Southern and RNA blots and as primers in amplification reactions, or for vaccines.

Suitable hybridization conditions will be well known to those of skill in the art. In certain applications, for example, substitution of amino acids by site-directed mutagenesis, it is appreciated that lower stringency conditions are required. Under these conditions, hybridization may occur even though the sequences of probe and target strand are not perfectly complementary, but are mismatched at one or more positions. Site-specific mutagenesis is a technique useful in the preparation of individual peptides, or biologically functional equivalent proteins or peptides, through specific mutagenesis of the underlying DNA. Typically, a primer of about 17 to 25 nucleotides in length is preferred, with about 5 to 10 residues on both sides of the junction of the sequence being altered (see Sambrook et al., 2001).

One method of using probes and primers of the present invention is in the search for genes related to the polynucleotides of Borrelia identified as encoding antigenic Borrelia polypeptides or, more particularly, homologues of Borrelia polypeptides from other related bacteria. Normally, the target DNA will be a genomic or cDNA library, although screening may involve analysis of RNA molecules. By varying the stringency of hybridization, and the region of the probe, different degrees of homology may be discovered (see Sambrook et al., 2001).

Another method of using oligonucleotides of the present invention is to design short RNA molecules for specific expression interference in vivo (sRNAi).

B. Polypeptides and Antigens

For the purposes of the present invention a Borrelia polypeptide, i.e., a polypeptide derived from a bacteria of the Borrelia genus, may be a naturally-occurring polypeptide that has been extracted using protein extraction techniques well known to those of skill in the art. In particular embodiments, an Borrelia antigen may be identified by ELI and prepared in a pharmaceutically acceptable carrier for the vaccination of an animal.

In alternative embodiments, the Borrelia polypeptide or antigen may be a synthetic peptide. In still other embodiments, the peptide may be a recombinant peptide produced through molecular engineering techniques. The present section describes the methods and compositions involved in producing a composition of Borrelia polypeptides for use as antigens in the present invention.

1. Borrelia Polypeptides

Methods for screening and identifying Borrelia genes that confer protection against Borrelia infection are described herein. The Borrelia polypeptide encoding genes or their corresponding cDNA may be inserted into an appropriate expression vector for the production of antigenic Borrelia polypeptides. In addition, sequence variants of the polypeptide may be prepared. Polypeptide sequence variants may be minor sequence variants of the polypeptide that arise due to natural variation within the population or they may be homologues found in other bacteria. There also may be sequences that do not occur naturally, but that are sufficiently similar that they function similarly and/or elicit an immune response that cross-reacts with natural forms of the polypeptide. Sequence variants can be prepared by standard methods of site-directed mutagenesis such as those described in Sambrook et al. 2001.

Another synthetic or recombinant variation of an antigenic Borrelia polypeptide is a polyepitop moiety comprising repeats of epitop determinants found naturally in Borrelia proteins. Such synthetic polyepitop proteins can be made up of several homomeric repeats of any one Borrelia protein epitope; or may comprise of two or more heteromeric epitopes expressed on one or several Borrelia protein epitopes.

Amino acid sequence variants of the polypeptide can be substitutional, insertional or deletion variants. Deletion variants lack one or more residues of the native protein which are not essential for function or immunogenic activity. Another common type of deletion variant is one lacking secretory signal sequences or signal sequences directing a protein to bind to a particular part of a cell.

Substitutional variants typically contain the exchange of one amino acid for another at one or more sites within the protein, and may be designed to modulate one or more properties of the polypeptide such as stability against proteolytic cleavage. Substitutions preferably are conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

Insertional variants include fusion proteins such as those used to allow rapid purification of the polypeptide and also can include hybrid proteins containing sequences from other proteins and polypeptides that are homologs of the polypeptide. For example, an insertional variant could include portions of the amino acid sequence of the polypeptide from one species, together with portions of the homologous polypeptide from another species or subspecies. Other insertional variants can include those in which additional amino acids are introduced within the coding sequence of the polypeptide. These typically are smaller insertions than the fusion proteins described above and are introduced, for example, into a protease cleavage site.

In one embodiment, major antigenic determinants of the polypeptide may be identified by an empirical approach in which portions of the gene encoding the polypeptide are expressed in a recombinant host, and the resulting proteins tested for their ability to elicit an immune response. For example, the polymerase chain reaction (PCR) can be used to prepare a range of cDNAs encoding peptides lacking successively longer fragments of the C-terminus of the protein. The immunogenic activity of each of these peptides then identifies those fragments or domains of the polypeptide that are essential for this activity. Further experiments in which only a small number of amino acids are removed or added at each iteration then allows the location of other antigenic determinants of the polypeptide to be determined. Thus, the polymerase chain reaction, a technique for amplifying a specific segment of DNA via multiple cycles of denaturation-renaturation, using a thermostable DNA polymerase, deoxyribonucleotides and primer sequences is contemplated in the present invention (Mullis, 1990; Mullis et al., 1992).

Another embodiment for the preparation of the polypeptides according to the invention is the use of peptide mimetics. Mimetics are molecules that mimic elements of protein secondary structure. Because many proteins exert their biological activity via relatively small regions of their folded surfaces, their actions can be reproduced by much smaller designer (mimetic) molecules that retain the bioactive surfaces and have potentially improved pharmacokinetic/dynamic properties (Fairlie et al., 1998). Methods for mimicking individual elements of secondary structure (helices, turns, strands, sheets) and for assembling their combinations into tertiary structures (helix bundles, multiple loops, helix-loop-helix motifs) have been reviewed (Fairlie et al., 1998; Moore, 1994). Methods for predicting, preparing, modifying, and screening mimetic peptides are described in U.S. Pat. Nos. 5,933,819 and 5,869,451 (each specifically incorporated herein by reference). It is contemplated in the present invention, that peptide mimetics will be useful in screening modulators of an immune response.

Modifications and changes may be made in the sequence of a gene or polynucleotide and still obtain a molecule that encodes a protein or polypeptide with desirable characteristics. The following is a discussion based upon changing the amino acids of a protein or polypeptide to create an equivalent, or even an improved, second-generation molecule. The amino acid changes may be achieved by changing the codons of the DNA sequence or by chemical peptide synthesis, according to the following examples.

For example, certain amino acids may be substituted for other amino acids in a polypeptide structure without appreciable loss of interactive binding capacity with structures such as, for example, antigen-binding regions of antibodies or binding sites on substrate molecules. Since it is the interactive capacity and nature of a polypeptide that defines the biological activity, certain amino acid substitutions can be made in a polypeptide sequence, and its underlying DNA coding sequence, and nevertheless obtain a polypeptide with like or improved properties. It is thus contemplated by the inventor that various changes may be made in the DNA sequences of the polynucleotides and genes of the invention without appreciable loss of their biological utility or activity. In some cases it is anticipated that modification may increase utility or activity. Table 1 shows the codons that encode particular amino acids.

In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It is known in the art that certain amino acids may be substituted by other amino acids having a similar hydropathic index or score and still result in a protein or polypeptide with similar biological activity. It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein.

It is also understood that an amino acid can be substituted for another having a similar hydrophilicity value and still obtain a biologically equivalent and immunologically equivalent protein.

Amino acid substitutions generally are based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutamine and asparagine; and valine, leucine and isoleucine, as well as others.

2. Synthetic Polypeptides

Contemplated in the present invention are Borrelia proteins and related peptides for use as antigens. In certain embodiments, the synthesis of an Borrelia peptide fragment is considered. The peptides of the invention can be synthesized in solution or on a solid support in accordance with conventional techniques. Various automatic synthesizers are commercially available and can be used in accordance with known protocols. See, for example, Stewart and Young, (1984); Tam et al., (1983); Merrifield, (1986); and Barany and Merrifield (1979), each incorporated herein by reference.

3. Polypeptide Purification

Borrelia polypeptides of the present invention are typically used as antigens for inducing a protective immune response in an animal and for the preparation of anti-Borrelia antibodies. Thus, certain aspects of the present invention concern the purification, and in particular embodiments, the substantial purification, of an Borrelia polypeptide. The term “purified protein or peptide ” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50% or more of the proteins in the composition.

Various methods for quantifying the degree of purification of the protein or peptide will be known to those of skill in the art in light of the present disclosure. These include, for example, determining the specific activity of an active fraction, or assessing the number of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulphate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. Methods exhibiting a lower degree of relative purification may have advantages in total recovery of protein product, or in maintaining the activity of an expressed protein.

To purify a desired protein, polypeptide, or peptide, which is a natural or recombinant composition comprising at least some specific proteins, polypeptides, or peptides will be subjected to fractionation to remove various other components from the composition. Various techniques suitable for use in protein purification will be well known to those of skill in the art. The most commonly used separative procedure for chemically synthesized peptides is HPLC chromatography. Other procedures for protein purification include affinity chromatography (e.g., immunoaffinity chromatography) and other methods known in the art. For exemplary methods and a more detailed discussion see Strategies for Protein Purification and Characterization: A Laboratory Course Manual, By Daniel R. Marshak et al., Cold Spring Harbor Laboratory, 1996, ISBN 0-87969-385-1 or Protein Purification: Principles, High-Resolution Methods, and Applications, 2nd Edition, Jan-Christer Janson, Lars Rydén, 1998, ISBN: 0-471-18626-0

C. Polynucleotide Delivery

In certain embodiments of the invention, an expression construct comprising an Borrelia polynucleotide or polynucleotide segment under the control of a heterologous promoter operable in eukaryotic cells is provided. For example, the delivery of an B. burgdorferi antigen-encoding expression constructs can be provided in this manner. The general approach in certain aspects of the present invention is to provide a cell with an expression construct encoding a specific protein, polypeptide or peptide fragment, thereby permitting the expression of the antigenic protein, polypeptide or peptide fragment in the cell. Following delivery of the expression construct, the protein, polypeptide or peptide fragment encoded by the expression construct is synthesized by the transcriptional and translational machinery of the cell and/or the vaccine vector. Various compositions and methods for polynucleotide delivery are known (see Sambrook et al., 2001; Liu and Huang, 2002; Ravid et al., 1998; Balicki and Beutler, 2002 and, each of which is incorporated herein by reference).

Viral and non-viral delivery systems are two of the various delivery systems for the delivery of an expression construct encoding an antigenic protein, polypeptide, polypeptide fragment. Both types of delivery systems are well known in the art and are briefly described below. There also are two primary approaches utilized in the delivery of an expression construct for the purposes of genetic immunization; either indirect, ex vivo methods or direct, in vivo methods. Ex vivo gene transfer comprises vector modification of (host) cells in culture and the administration or transplantation of the vector modified cells to a subject. In vivo gene transfer comprises direct introduction of the vaccine vector into the subject to be immunized.

In various embodiments, a nucleic acid to be expressed may be in the context of a linear expression elements (“LEEs”) and/or circular expression elements (“CEEs”), which typically encompass a complete set of gene expression components (promoter, coding sequence, and terminator). These LEEs and CEEs can be directly introduced into and expressed in cells or an intact organism to yield expression levels comparable to those from a standard supercoiled, replicative plasmid (Sykes and Johnston, 1999). In some alternative methods and compositions of the invention, LEE or CEE allows any open-reading frame (ORF), for example, PCR™ amplified ORFs, to be non-covalently linked to an eukaryotic promoter and terminator. These quickly linked fragments can be directly injected into animals to produce local gene expression. It has also been demonstrated that the ORFs can be injected into mice to produce antibodies to the encoded foreign protein by simply attaching mammalian promoter and terminator sequences.

In certain embodiments of the invention, the nucleic acid encoding Borrelia or similar polynucleotide may be stably integrated into the genome of a cell. In yet further embodiments, the nucleic acid may be stably or transiently maintained in a cell as a separate, episomal segment of DNA. Such nucleic acid segments or “episomes” encode sequences sufficient to permit maintenance and replication independent of or in synchronization with the host cell cycle. How the expression construct is delivered to a cell and/or where in the cell the nucleic acid remains is dependent on the type of vector employed. The following gene delivery methods provide the framework for choosing and developing the most appropriate gene delivery system for a preferred application.

1. Non-Viral Polynucleotide Delivery

In one embodiment of the invention, a polynucleotide expression construct may include recombinantly-produced DNA plasmids or in vitro-generated DNA. In various embodiments of the invention, an expression construct comprising, for example, a Borrelia polynucleotide is administered to a subject via injection and/or particle bombardment (e.g., a gene gun). Polynucleotide expression constructs may be transferred into cells by accelerating DNA-coated microprojectiles to a high velocity, allowing the DNA-coated microprojectiles to pierce cell membranes and enter cells. In another preferred embodiment, polynucleotides are administered to a subject by needle injection. A polynucleotide expression construct may be given by intramuscular, intravenous, subcutaneous, intradermal, or intraperitoneal injection.

Particle Bombardment depends on the ability to accelerate DNA-coated microprojectiles to a high velocity allowing them to pierce cell membranes and enter cells without killing them (Klein et al., 1987). Several devices for accelerating small particles have been developed. The most commonly used forms rely on high-pressure helium gas (Sanford et al., 1991). The microprojectiles used have consisted of biologically inert substances such as tungsten or gold beads.

Transfer of an expression construct comprising Borrelia or similar polynucleotides of the present invention also may be performed by any of the methods which physically or chemically permeabilize the cell membrane (e.g., calcium phosphate precipitation, DEAE-dextran, electroporation, direct microinjection, DNA-loaded liposomes and lipofectamine-DNA complexes, cell sonication, gene bombardment using high velocity microprojectiles and receptor-mediated transfection. In certain embodiments, the use of lipid formulations and/or nanocapsules is contemplated for the introduction of a Borrelia polynucleotide, Borrelia polypeptide, or an expression vector comprising a Borrelia polynucleotide into host cells (see exemplary methods and compositions in Bangham et al. (1965), DRUG CARRIERS IN BIOLOGY AND MEDICINE, G. Gregoriadis ed. (1979) Deamer and Uster (1983), Szoka and Papahadjopoulos (1978), Nicolau et al., 1987 and Watt et al., 1986; each of which is incorporated herein by reference). In another embodiment of the invention, the expression construct may simply consist of naked recombinant DNA, expression cassettes or plasmids.

2. Viral Vectors

In certain embodiments, it is contemplated that a Borrelia gene or other polynucleotide that confers immune resistance to infection pursuant to the invention may be delivered by a viral vector. The capacity of certain viral vectors to efficiently infect or enter cells, to integrate into a host cell genome and stably express viral genes, have led to the development and application of a number of different viral vector systems (Robbins et al., 1998). Viral systems are currently being developed for use as vectors for ex vivo and in vivo gene transfer. For example, adenovirus, herpes-simple virus, retrovirus and adeno-associated virus vectors are being evaluated currently for treatment of diseases such as cancer, cystic fibrosis, Gaucher disease, renal disease and arthritis (Robbins and Ghivizzani, 1998; Imai et al., 1998; U.S. Pat. No. 5,670,488).

In particular embodiments, an adenoviral (U.S. Pat. Nos. 6,383,795, 6,328,958 and 6,287,571 each specifically incorporated herein by reference), retroviral (U.S. Pat. Nos. 5,955,331; 5,888,502, 5,830,725 each specifically incorporated herein by reference), Herpes-Simplex Viral (U.S. Pat. Nos. 5,879,934; 5,851,826, each specifically incorporated herein by reference in its entirety), Adeno-associated virus (AAV), poxvirus; e.g., vaccinia virus (Gnant et al., 1999), alpha virus; e.g., sindbis virus, Semliki forest virus (Lundstrom, 1999), reovirus (Coffey et al., 1998) and influenza A virus (Neumann et al., 1999), Chimeric poxyiral/retroviral vectors (Holzer et al., 1999), adenoviral/retroviral vectors (Feng et al., 1997; Bilbao et al., 1997; Caplen et al., 1999) and adenoviral/adeno-associated viral vectors (Fisher et al., 1996; U.S. Pat. No. 5,871,982), expression vectors are contemplated for the delivery of expression constructs. “Viral expression vector” is meant to include those constructs containing virus sequences sufficient to (a) support packaging of the construct and (b) to ultimately express a tissue or cell-specific construct that has been cloned therein. Virus growth and manipulation is known to those of skilled in the art.

D. Antibodies Reactive to Borrelia Antigens.

In another aspect, the present invention includes antibody compositions that are immunoreactive with a Borrelia polypeptide of the present invention, or any portion thereof. In still other embodiments, an antigen of the invention may be used to produce antibodies and/or antibody compositions. Antibodies may be specifically or preferentially reactive to Borrelia polypeptides. Antibodies reactive to Borrelia includes antibodies reactive to Borrelia polypeptides or polynucleotides encoding Borrelia polypeptides, including those directed against an antigen having the sequences as set forth in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, SEQ ID NO:112, SEQ ID NO:114, SEQ ID NO:117, SEQ ID NO:119, SEQ ID NO:121, SEQ ID NO:123, SEQ ID NO:125, SEQ ID NO:127, SEQ ID NO:129, SEQ ID NO:131, SEQ ID NO:133, SEQ ID NO:135, SEQ ID NO:137, SEQ ID NO:139, fragments, variants, or mimetics thereof, or closely related sequences. The antigens of SEQ ID NO:2, SEQ ID NO:6, SEQ ID NO:10, SEQ ID NO:14, SEQ ID NO:18, SEQ ID NO:22, SEQ ID NO:26, SEQ ID NO:30, SEQ ID NO:34, SEQ ID NO:38, SEQ ID NO:42, SEQ ID NO:46, SEQ ID NO:50, SEQ ID NO:54, SEQ ID NO:58, SEQ ID NO:62, SEQ ID NO:66, SEQ ID NO:70, SEQ ID NO:74, SEQ ID NO:78, SEQ ID NO:82, SEQ ID NO:86, SEQ ID NO:90, SEQ ID NO:94, SEQ ID NO:98, SEQ ID NO:102, SEQ ID NO:106, SEQ ID NO:110, SEQ ID NO:119, SEQ ID NO:123, SEQ ID NO:125, SEQ ID NO:129, SEQ ID NO:133, SEQ ID NO:137 are representative of antigenic fragments of Borrelia polypeptides. Antigens represented in SEQ ID NO:4, SEQ ID NO:8, SEQ ID NO:12, SEQ ID NO:16, SEQ ID NO:20, SEQ ID NO:24, SEQ ID NO:28, SEQ ID NO:32, SEQ ID NO:36, SEQ ID NO:40, SEQ ID NO:44, SEQ ID NO:48, SEQ ID NO:52, SEQ ID NO:56, SEQ ID NO:60, SEQ ID NO:64, SEQ ID NO:68, SEQ ID NO:72, SEQ ID NO:76, SEQ ID NO:80, SEQ ID NO:84, SEQ ID NO:88, SEQ ID NO:92, SEQ ID NO:96, SEQ ID NO:100, SEQ ID NO:104, SEQ ID NO:108, SEQ ID NO:112, SEQ ID NO:114, SEQ ID NO:117, SEQ ID NO:121, SEQ ID NO:127, SEQ ID NO:131, SEQ ID NO:135, and SEQ ID NO:139 are exemplary of full length Borrelia peptides from which exemplary antigenic fragments have been identified. The antibodies may be polyclonal or monoclonal and produced by methods known in the art. The antibodies may also be monovalent or bivalent. An antibody may be split by a variety of biological or chemical means. Each half of the antibody can only bind one antigen and, therefore, is defined monovalent. Means for preparing and characterizing antibodies are well known in the art (see, e.g., Howell and Lane, 1988, which is incorporated herein by reference).

Peptides corresponding to one or more antigenic determinants of a Borrelia polypeptide of the present invention may be prepared in order to produce an antibody. Such peptides should generally be at least five or six amino acid residues in length, will preferably be about 10, 15, 20, 25 or about 30 amino acid residues in length, and may contain up to about 35 to 50 residues or so. Synthetic peptides will generally be about 35 residues long, which is the approximate upper length limit of automated peptide synthesis machines, such as those available from Applied Biosystems (Foster City, Calif.). Longer peptides also may be prepared, e.g., by recombinant means. In other methods full or substantially full length polypeptides may be used to produce antibodies of the invention.

Once a peptide(s) are prepared that contain at least one or more antigenic determinants, the peptides are then employed in the generation of antisera against the polypeptide. Minigenes or gene fusions encoding these determinants also can be constructed and inserted into expression vectors by standard methods, for example, using PCR cloning methodology. The use of peptides for antibody generation or vaccination typically requires conjugation of the peptide to an immunogenic carrier protein, such as hepatitis B surface antigen, keyhole limpet hemocyanin or bovine serum albumin. Methods for performing this conjugation are well known in the art.

The antibodies used in the methods of the invention include derivatives that are modified, i.e, by the covalent attachment of any type of molecule to the antibody such that covalent attachment For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand (a other protein, etc. Any of numerous chemical modifications may be carried out by known techniques, including, but cot limited to, specific chemical cleavage, acetylation, formylation metabolic synthesis tunicamycin, etc. Additionally, the derivative may contain one or more non-classical ammo acids.

For some uses, including in vivo use of antibodies in humans and in vitro detection assays, it may be preferable to use chimeric, humanized, or human antibodies. A dimeric antibody is a molecule in which different portions of the antibody are derived from different animal species, such as antibodies having a variable region derived from a murine monoclonal antibody and a constant region derived, from a human immunoglobulin. Methods for producing chimeric antibodies are known in the art. See e.g., Morrison, 1985; Ol et al., 1986; Gillies et al. 1989; U.S. Pat. Nos. 5,807,715; 4,816,567; and 4,816,397, which are incorporated herein by reference in their entireties. Humanized antibodies are antibody molecules from non-human species that bind the desired antigen having one or more complementarity determining regions (CDRs) from the non-human species and framework regions from a human immunoglobulin molecule. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. See, e.g., U.S. Pat. No. 5,585,089 and Riechmann et al. (1988), which are incorporated herein by reference in their entireties. Antibodies can be humanized using a variety of techniques known in the art including, for example, CDR-grafting (EP 239,400; WO 91/09967; U.S. Pat. Nos. 5,225,539; 5,530,101 and 5,585,089), veneering or resurfacing (EP 592,106; EP 519,596; Padlan, 1991; Studnicka et al., 1994; Roguska et al., 1994), and chain shuffling (U.S. Pat. No. 5,565,332), all of which are hereby incorporated by reference in their entireties.

Completely human antibodies are particularly desirable for therapeutic treatment of human patients. Human antibodies can be made by a variety of methods known in the art including phage display methods described above using antibody libraries derived from human immunoglobulin sequences. See U.S. Pat. Nos. 4,444,887 and 4,710,111; and WO 98/46645; WO 99/50433; WO 98/24893; WO 98/16654; WO 96/34096; WO 96/33735; and WO 91/10741, each of which is incorporated herein by reference in its entirety.

Human antibodies can also be produced using transgenic mice which are incapable of expressing functional endogenous immunoglobulins, but which can express human immunoglobulin genes. For an overview of this technology for producing human antibodies, see Lonberg and Huszar (1995). For a detailed discussion of this technology for producing human antibodies and human monoclonal antibodies and protocols for producing such antibodies, see, e.g., WO 98/24893; WO 92/01047; WO 96/34096; WO 96/33735; EP 0598877; U.S. Pat. Nos. 5,413,923; 5,625,126; 5,633,425; 5,569,825; 5,661,016; 5,545,806; 5,814,318; 5,885,793; 5,916,771; and 5,939,598; which are incorporated by reference herein in their entireties. In addition, companies such as Abgenix, Inc. (Freemont, Calif.). Kirin, Inc. (Japan), Medarex (NJ) and Genpharm (San Jose, Calif.) can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope (Jespers et al., 1988).

The present invention encompasses single domain antibodies, including camelized single domain antibodies (See e.g., Muyldermans et al., 2001; Nuttall et al., 2000; Reichmann and Muyldermans, 1999; WO 94/04678; WO 94/25591; and U.S. Pat. No. 6,005,079; which are incorporated herein by reference in their entireties), In one embodiment, the present invention provides single domain antibodies comprising two VH domains with modifications such that single domain antibodies are formed.

The methods of the present invention also encompass the use of antibodies or fragments thereof that have half-lives (e.g., serum half-lives) in a mammal, preferably a human, of greater than 15 days, preferably greater than 20 days, greater than 25 days, greater than 30 days, greater, than 35 days, greater than 40 days, greater than 45 days, greater than 2 months, greater than 3 months, greater than 4 months, or greater than 5 months. The increased half-lives of the antibodies of the present invention or fragments thereof in a mammal, preferably a human, results in a higher serum titer of said antibodies or antibody fragments in the mammal, and thus, reduces the frequency of the administration of said antibodies or antibody fragments and/or reduces the concentration of said antibodies or antibody fragments to be administered. Antibodies or fragments thereof having increased in vivo half-lives can be generated by techniques known to those of skill in the art. For example, antibodies or fragments thereof will increased in vivo half-lives can be generated by modifying (e.g., substituting, deleting or adding) amino acid residues identified as involved in the interaction between the Fc domain and the FcRn receptor. The antibodies of the invention may be engineered by methods described in Ward et al. to increase biological half-lives (see U.S. Pat. No. 6,277,375 B1). For example, antibodies of the invention maybe engineered in the Fc-hinge domain to have increased in vivo or serum half-lives.

Antibodies or fragments thereof with increased in vivo half-lives can be generated by attaching to said antibodies or antibody fragments polymer molecules such as high molecular weight polyethyleneglycol (PEG). PEG can be attached to said antibodies or antibody fragments with or without a multifunctional linker either through site-specific conjugation of the PEG to the N- or C-terminus of said antibodies or antibody fragments or via episilon-amino groups present on lysine residues. Linear or branched polymer derivatization that results in minimal loss of biological activity will typically be used. The degree of conjugation will be closely monitored by SDS-PAGE and mass spectrometry to ensure proper conjugation of PEG molecules to the antibodies. Unreacted PEG can be separated from antibody-PEG conjugates by, e.g., size exclusion or ion-exchange chromatography.

The antibodies of the invention may also be modified by the methods and coupling agents described by Davis et al. (U.S. Pat. No. 4,179,337) in order to provide compositions that can be injected into the mammalian circulatory system with substantially no immunogenic response.

In one aspect, the invention features multispecific, multivalent molecules, which minimally comprise an anti-Fc receptor portion, an anti-target portion and optionally an anti-enhancement factor (anti-EF) portion. In preferred embodiments, the anti-Fc receptor portion is an antibody fragment (e.g., Fab or (Fab′)₂ fragment), the anti-target portion is a ligand or antibody fragment and the anti-EF portion is an antibody directed against a surface protein involved in cytotoxic activity. In a particular embodiment, the recombinant anti-FcR antibodies, or fragments are “humanized” (e.g., have at least a portion of a complementarity determining region (CDR) derived from a non-human antibody (e.g., murine) with the remaining portion(s) being human in origin).

In various embodiments, the invention includes methods for generating multispecific molecules, e.g., a first specificity for an antigen and a second specificity for a Fc receptor. In one embodiment, both specificities are encoded in the same vector and are expressed and assembled in a host cell. In another embodiment, each specificity is generated recombinantly and the resulting proteins or peptides are conjugated to one another via sulfhydryl bonding of the C-terminus hinge regions of the heavy chain. In a particularly preferred embodiment, the hinge region is modified to contain only one sulfhydryl residue, prior to conjugation. For examples of these and other related methods and compositions see U.S. Pat. Nos. 6,410,690; 6,365,161; 6,303,755; 6,270,765; and 6,258,358 each of which are incorporated herein by reference.

The invention also encompasses the use of antibodies or antibody fragments comprising the amino acid sequence of any of the antibodies of the invention with mutations (e.g., one or more amino acid substitutions) in the framework or variable regions. Preferably, mutations in these antibodies maintain or enhance the avidity and/or affinity of the antibodies for the particular antigen(s) to which they immunospecifically bind. Standard techniques known to those skilled in the art (e.g., immunoassays) can be used to assay the affinity of an antibody for a particular antigen.

The present invention also encompasses antibodies comprising a modified Fc region. Modifications that affect Fc-mediated effector function are well known in the art (U.S. Pat. No. 6,194,551, which is incorporated herein by reference in its entirety), for example, one or more ammo acids alterations (e.g., substitutions) are introduced in the Fc region. The ammo acids modified can be, for example, Proline 329, Proline 331, or Lysine 322. Proline 329, 331 and Lysine 322 are preferably replaced with alanine, however, substitution with any other amino acid is contemplated. WO 00/42072 and U.S. Pat. No. 6,194,551, which are incorporated herein by reference. In one particular embodiment, the modification of the Fc region comprises one or more mutations in the Fc region. In another particular embodiment, the modification in the Fc region has altered antibody-mediated effector function. In another embodiment of the invention, the modification in the Fc region has altered binding to other Fc receptors (e.g., Fc activation receptors). In yet another particular embodiment, the antibodies of the invention comprising a modified Fc region mediate ADCC more effectively. In another embodiment, the modification in the Fc region alters C1q binding activity. In yet a further embodiment, the modification in the Fc region alters complement dependant cytotoxicity.

The invention also comprises antibodies with altered carbohydrate modifications (e.g., glycosylation, fusocylation, etc.), wherein such modification enhances antibody-mediated effector function. Carbohydrate modifications that lead to altered antibody mediated effector function are well known in the art (for example see Shields et al., 2001; Davies et al., 2001).

1. Antibody Conjugates

The present invention encompasses antibodies recombinantly fused or chemically conjugated (including both covalently and non-covalent conjugations) to heterologous polypeptides (i.e., an unrelated polypeptide; or portion thereof, preferably at least 10, at least 20, at least 30, at least 40, at least 50, at least 60, at least 70, at least 80, at least 90, or at least 100 amino acids of the polypeptide) to generate fusion proteins. The fusion does not necessarily need to be direct, but may occur through linker sequences. Antibodies may be used for example to target heterologous polypeptides to particular cell types, either in vitro or in vivo, by fusing or conjugating the antibodies to antibodies specific for particular cell surface receptors. Antibodies fused or conjugated to heterologous polypeptides may also be used in in vitro immunoassays and purification methods using methods known in, the art. See e.g., WO 93/21232; EP 439,095; Naramura et al., 1994; U.S. Pat. No. 5,474,981; Gillies et al., 1992; and Fell et al., 1991, which are incorporated herein by reference in their entireties.

Further, an antibody may be conjugated to a therapeutic agent or drug moiety that modifies a given biological response. Therapeutic agents or drug moieties are not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin (i.e., PE-40), or diphtheria toxin, ricin, gelonon, and pokeweed antiviral protein, a protein such as tumor necrosis factor, interferons including, but not limited to, alpha-interferon (IFN-α), beta-interferon (IFN-β), nerve growth factor (NGF), platelet derived growth factor (PDGF), tissue plasminogen activator (TPA), an apoptotic agent (e.g., TNF-α, TNF-β, AIM I (as disclosed in WO 97/33899), AIM II (WO 97/34911), Fas Ligand (Takahashi et al., 1994), and VEGI (WO 99/23105), a thrombotic agent or an anti-angiogenic agent (e.g., angiostain or endostatin), or a biological response modifier such as, for example, lymphokine (e.g., interleukm-1 (“IL-1”), interleukin-2 (“IL-2”), interleukm-6 (“IL-6”) granulocyte macrophage colony stimulating factor (“GM-CSF”), and granulocyte colony stimulating factor (“G-CSF”), macrophage colony stimulating factor, (“M-CSF”), or a growth factor (e.g., growth hormone (“GH”); proteases, or ribonucleases.

Antibodies can be fused to marker sequences, such as a peptide to facilitate purification. In preferred embodiments, the marker amino acid sequence is a hexa-histidine peptide, such as the tag provided in a pQE vector (QIAGEN, Inc., Chatsworth, Calif.), among others, many of which are commercially available. As described in Gentz et al., 1989, for instance, hexa-histidine provides for convenient purification of the fusion protein. Other peptide tags useful for purification include, but are not limited to, the hemagglutinin “HA” tag, which corresponds to an epitope derived nom the influenza hemagglutinin protein (Wilson et al., 1984) and the “flag” tag (Knappik et al., 1994).

The present invention further includes compositions comprising heterologous polypeptides fused or conjugated to antibody fragments. For example, the heterologous polypeptides may be fused or conjugated to a Fab fragment, Fd fragment, Fv fragment, F(ab)₂ fragment, or portion thereof. Methods for fusing or conjugating polypeptides to antibody portions are known in the art. See for example U.S. Pat. Nos. 5,336,603; 5,622,929; 5,359,046; 5,349,053; 3,447,851; and 5,112,946; EP 307,434; EP 367,166; WO 96/04388 and WO 91/06570; Ashkenazi et al., 1991; Zheng et al., 1995; and Vil et al., 1992; each of which are incorporated by reference in there entireties).

Additional fusion proteins may be generated through the techniques of gene-shuffling, motif-shuffling, exon-shuffling; and/or codon-shuffling (collectively referred to as “DNA shuffling”). DNA shuffling may be employed to alter the activities of antibodies of the invention or fragments thereof (e.g., antibodies or fragments thereof with higher affinities and lower dissociation rates). See, generally, U.S. Pat. Nos. 5,605,793; 5,811,238; 5,830,721; 5,834,252; and 5,837,458; and Patten et al., 1997; Harayama, 1998; Hansson et al., 1999; Lorenzo and Blasco, 1998; each of which are hereby incorporated by reference in its entirety. Antibodies or fragments thereof, or the encoded antibodies or fragments thereof, may be altered by being subjected to random mutagenesis by error-prone PCR, random nucleotide insertion or other methods prior to recombination. One or more portions of a polynucleotide encoding an antibody or antibody fragment, which portions specifically bind to FcγRIIB may be recombined with one or more components, motifs, sections, parts, domains, fragments, etc. of one or more heterologous molecules.

The present invention also encompasses antibodies conjugated to a diagnostic or therapeutic agent or any other molecule for which serum half-life is desired to be increased. The antibodies can be used diagnostically to, for example, monitor the development or progression of a disease, disorder or infection as part of a clinical testing procedure to, e.g., determine the efficacy of a given treatment regimen. Detection can be facilitated by coupling an antibody or an antigen to a detectable substance. Examples of detectable substances include various enzymes, prosthetic groups, fluorescent materials, luminescent materials, bioluminescent materials, radioactive materials, positron emitting metals, and non-radioactive paramagnetic metal ions. The detectable substance may be coupled or conjugated either directly to the antibody or antigen or indirectly, through an intermediate (such as, for example, a linker known in the art) using techniques known in the art, See, for example, U.S. Pat. No. 4,741,900 for metal ions which can be conjugated to antibodies for use as diagnostics according to the present invention. Such diagnosis and detection can be accomplished by coupling the antibody or antigen to detectable substances including, but not limited to, various enzyme, enzymes including, but not limited to, horseradish peroxidase, alkaline phosphatase, beta-galactosidase, or acetylcholinesterase; prosthetic group complexes such as, but not limited to, streptavidin/biotin and avidin/biotin; fluorescent materials such as, but not limited to umbelliferone, fluorescein, fluorescein isothiocyanate, rhodamine, dichlorotriazinylamine, fluorescein, dansyl chloride or phycoerythrin; luminescent material such as, but not limited to, luminol; bioluminescent materials such as, but not limited to, luciferase, luciferin, and aequorin; radioactive material such as, but not limited to, bismuth (²¹³B), carbon (¹⁴C), chromium (⁵¹Cr), cobalt (⁵⁷Co), fluorine (¹⁸F), gadolinium (¹⁵³Gd, ¹⁵⁹Gd), gallium (⁶⁸Ga, ⁶⁷Ga), germanium (⁶⁸Ge), holmium (¹⁶⁶Ho), indium (¹¹⁵In, ¹¹³In, ¹¹²In, ¹¹¹In), iodine (¹³¹I, ¹²⁵I, ¹²³I, ¹²¹I) lansthanium (¹⁴⁰La), lutetitium (¹⁷⁷Lu), manganese (⁵⁴Mn), molybdenum (⁹⁹Mo), palladium (¹⁰³Pd), phosphorous (³²p), praseodymium (¹⁴²Pr), promethium (149 Pm), rhenium (¹⁸⁶Re, ¹⁸⁸Re), rhodium (¹⁰⁵Rh), ruthemium (⁹⁷Ru), samarium (¹⁵³Sm), scandium (⁴⁷Sc), selenium (⁷⁵Se), strontium (⁸⁵Sr), sulfur (³⁵S), technetium (⁹⁹Tc), titanium (⁴⁴Ti), tin (¹¹³Sn, ¹¹⁷Sn), tritium (³H), xenon (¹³⁶ Xe), ytterbium (¹⁷⁹Yb, ¹⁷⁵Yb), yttrium (⁹⁰Y), zinc (⁶⁵Zn); positron emitting metals using various positron emission tomographies, and non-radioactive paramagnetic metal ions.

An antibody may be conjugated to a therapeutic moiety such as a. cytotoxin (e.g., a cytostatic or cytocidal agent), a therapeutic agent or a radioactive element (e.g., alpha-emitters, gamma-emitters, etc.). Cytotoxins or cytotoxic agents include any agent that is detrimental to cells. Examples include paclitaxol, cytochalasin B, gramicidin D, ethidium bromide, emetine, mitomycin, etoposide, tenoposide, vincristine, vinblastine, colchicin, doxorubicin, daunorubicin, dihydroxy anrhracindione, mitoxantrone. mithramycin, actinciomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine; cytarabine, 5-fluorouracil decarbazine), alkylating agents (e.g., mechlorethamine, thioepa Chlorambucil, melphalan, carmustine (BSNU) and lomustine (CCNU), cyclophosphamide, busulfan, dibromomannitol, streptozotocin, mitomycin C, and cisdichlorodiamine platinum (II) (DDP) cisplatin.), anthracyclines (e.g., daunorubicin (formerly daunomycin) and doxorubicin). antibiotics (e.g., dactinomycin (formerly actinomycin), bleomycin, mithramycin, and anthramycin (AMC)), and anti-mitotic agents (e.g., vincristine and vinblastine).

Moreover, an antibody can be conjugated to therapeutic moieties such as a radioactive materials or macrocyclic chelators useful for conjugating radiometal ions (see above for examples radioactive materials). In certain embodiments, macrocyclic chelator is 1,4,7,10-tetraazacyclododecane-N,N′,N″,N′″-tetraacetic acid (DOTA) which can be attached to the antibody via a linker molecule. Such linker molecules are commonly known in the art and described in Denardo et al., 1998; Peterson et al., 1999; and Zimmerman et al., 1999, each incorporated by reference in their entireties.

Techniques for conjugating such therapeutic moieties to antibodies are well known; see, example Arnon et al., 1985; Hellstrom et al., 1987; Thorpe, 1985; Thorpe et al., 1982.

An antibody or fragment thereof, with or without a therapeutic moiety conjugated to it, administered alone or in combination with cytotoxic factor(s) and/or cytokine(s) can be used as a therapeutic.

Alternatively, an antibody can be conjugated to a second antibody to form an antibody heteroconjugate as described by Segal (U.S. Pat. No. 4,676,980, which is incorporated herein by reference in its entirety.

Antibodies may also be attached to solid supports, which are particularly useful for immunoassays or purification, of the target antigen. Such solid supports include, but are not limited to, glass, cellulose, polyacrylamide, nylon, polystyrene, polyvinyl chloride or polypropylene.

2. Anti-Borrelia Antibody Generation

The present invention provides monoclonal antibody compositions that are immunoreactive with a Borrelia polypeptide. As detailed above, in addition to antibodies generated against a full length Borrelia polypeptide, antibodies also may be generated in response to smaller constructs comprising epitopic core regions, including wild-type and mutant epitopes. In other embodiments of the invention, the use of anti-Borrelia single chain antibodies, chimeric antibodies, diabodies and the like are contemplated.

As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting.

However, “humanized” Borrelia antibodies also are contemplated, as are chimeric antibodies from mouse, rat, goat or other species, fusion proteins, single chain antibodies, diabodies, bispecific antibodies, and other engineered antibodies and fragments thereof. As defined herein, a “humanized” antibody comprises constant regions from a human antibody gene and variable regions from a non-human antibody gene. A “chimeric antibody, comprises constant and variable regions from two genetically distinct individuals. An anti-Borrelia humanized or chimeric antibody can be genetically engineered to comprise an Borrelia antigen binding site of a given of molecular weight and biological lifetime, as long as the antibody retains its Borrelia antigen binding site. Humanized antibodies may be prepared by using following the teachings of U.S. Pat. No. 5,889,157

The term “antibody” is used to refer to any antibody-like molecule that has an antigen binding region, and includes antibody fragments such as Fab′, Fab, F(ab′)₂, single domain antibodies (DABs), Fv, scFv (single chain Fv), chimeras and the like. Methods and techniques of producing the above antibody-based constructs and fragments are well known in the art (U.S. Pat. Nos. 5,889,157; 5,821,333; 5,888,773, each specifically incorporated herein by reference). The methods and techniques for preparing and characterizing antibodies are well known in the art (See, e.g., Antibodies: A Laboratory Manual, Cold Spring Harbor Laboratory, 1988; incorporated herein by reference).

As also well known in the art, the immunogenicity of a particular immunogen composition can be enhanced by the use of non-specific stimulators of the immune response, known as adjuvants. Suitable molecule adjuvants include all acceptable immunostimulatory compounds, such as cytokines, toxins or synthetic compositions. In addition to adjuvants, it may be desirable to coadminister biologic response modifiers (BRM), which have been shown to upregulate T cell immunity or down-regulate suppressor cell activity.

2. Detecting Borrelia The invention also relates to methods of assaying for the presence of Borrelia infection, in particular B. burgdorferi or B. afzelii infection, in a vertebrate animal comprising: (a) obtaining an antibody directed against a Borrelia antigen; (b) obtaining a sample from the animal; (c) admixing the antibody with the sample; and (d) assaying the sample for antigen-antibody binding, wherein the antigen-antibody binding indicates Borrelia infection in the animal. In some cases, the antibody directed against the antigen is further defined as a polyclonal antibody. In other embodiments, an antibody directed against the antigen is further defined as a monoclonal antibody. In some embodiments, an antibody is reactive against an antigen having a sequence as set forth in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:10, SEQ ID NO:112, SEQ ID NO:114, SEQ ID NO:117, SEQ ID NO:119, SEQ ID NO:121, SEQ ID NO:123, SEQ ID NO:125, SEQ ID NO:127, SEQ ID NO:129, SEQ ID NO:131, SEQ ID NO:133, SEQ ID NO:135, SEQ ID NO:137, SEQ ID NO:139, fragments, variants, or mimetics thereof, or closely related sequences. The assaying of the sample for antigen-antibody binding may be by precipitation reaction, radioimmunoassay, ELISA, Western blot, immunofluorescence, or any other method known to those of skill in the art.

In other embodiments, the invention also relates to methods of assaying for the presence of Borrelia infection or antibodies reactive to Borrelia in a patient, subject, vertebrate animal, and/or human comprising: (a) obtaining a peptide, as described above; (b) obtaining a sample from a subject, patient, and/or animal; (c) admixing the peptide with the sample; and (d) assaying the sample for antigen-antibody binding, wherein the antigen-antibody binding indicates exposure of the animal to Borrelia. The peptide or antigen may have a sequence as set forth in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, SEQ ID NO:112, SEQ ID NO:114, SEQ ID NO:117, SEQ ID NO:119, SEQ ID NO:121, SEQ ID NO:123, SEQ ID NO:125, SEQ ID NO:127, SEQ ID NO:129, SEQ ID NO:131, SEQ ID NO:133, SEQ ID NO:135, SEQ ID NO:137, SEQ ID NO:139, fragments, variants, or mimetics thereof, or closely related sequences. The assaying of the sample for antigen-antibody binding may be by precipitation reaction, radioimmunoassay, ELISA, Western blot, immunofluorescence, or any other method known to those of skill in the art.

The invention further relates to methods of assaying for the presence of an Borrelia infection in an animal comprising: (a) obtaining an oligonucleotide probe comprising a sequence comprised within one of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:101, SEQ ID NO:103, SEQ ID NO:105, SEQ ID NO:107, SEQ ID NO:109, SEQ ID NO:111, SEQ ID NO:113, SEQ ID NO:115, SEQ ID NO:116, SEQ ID NO:118, SEQ ID NO:120, SEQ ID NO:122, SEQ ID NO:124, SEQ ID NO:126, SEQ ID NO:128, SEQ ID NO:130, SEQ ID NO:132, SEQ ID NO:134, SEQ ID NO:136, or SEQ ID NO:138, a complement, a fragment, or a closely related sequences thereof; and (b) employing the probe in a PCR or other detection protocol.

E. Other Binding or Affinity Agents

Various embodiments of the invention may include the use of alternative binding or affinity agents that preferentially bind nucleic acids and/or polypeptides, including fragments, portions, subdivisions and the like, of nucleic acids or polypeptides, including variants thereof, of the present invention. A binding agent may include nucleic acids, amino acids, synthetic polymers, carbohydrates, lipids, and combinations thereof as long as the compound, molecule, or complex preferentially binds or has a measurable affinity, as determined by methods known in the art, for a nucleic acid or polypeptide of the present invention. The binding affinity of an agent can, for example, be determined by the Scatchard analysis of Munson and Pollard, (1980). Other binding agents may include, but are not limited to nucleic acid aptamers; anticalins or other lipocalin derivatives (for examples see U.S. Pat. Nos. 5,506,121; 6,103,493 and WO 99/16873, WO 00/75308 and the like); or synthetic or recombinant antibody derivatives (for examples see U.S. Pat. No. 6,136,313) Exemplary methods and compositions may be found in U.S. Pat. Nos. 5,506,121; 6,103,493 and WO 99/16873, WO 00/75308 and the like, each of which is incorporated herein by reference. Any binding or affinity agents derived using the compositions of the present invention may be used in therapeutic, prophylactic, vaccination and/or diagnostic methods.

V. Therapeutic Compositions and Methods

It is further contemplated that the compositions and methods of the invention may be used as a therapeutic composition for bacterial infections. The therapeutics may be used to treat and/or diagnose viral infection. In certain embodiments, the nucleic acid and/or polypeptides of the invention may be used as a therapeutic agent. In various embodiments of the invention antibodies, binding agents, or affinity agents that recognize and/bind the nucleic acids or polypeptides of the invention may be used as therapeutic agents. These therapeutic compositions may act through mechanisms that include, but are not limited to the induction or stimulation of an active immune response by an organism or subject. Such therapeutic methods include passive immunization, prime-boost immunization, and other methods of using antigens, vaccines, and/or antibodies or other binding agents to protect, prevent, and/or treat infection by a pathogen.

Antibodies or binding agents of the invention may be conjugated to a therapeutic agent. Therapeutic agents may include, but are not limited to apoptosis-inducing agents, toxins, anti-viral agents, pro-drug converting enzymes and any other therapeutic agent that may aid in the treatment of a bacterial infection(s). Compositions of the present invention may be used in the targeting of a therapeutic agent to a focus of infection or to a pathogen, the method of which may include injecting a patient infected with a pathogen with an effective amount of an antibody-therapeutic agent conjugate. The conjugate may include an immunoreactive composite of one or more chemically-linked antibodies or antibody fragments which specifically binds to a one or more epitopes of one or more pathogens or of an antigen induced by the pathogen or presented by a cell as a result of the fragmentation or destruction of the pathogen at the focus of infection. The antibody conjugate may have a chemically bound therapeutic agent for treating said infection, thus localizing or targeting a therapeutic to the location of a pathogen.

Reviews of antimicrobial chemotherapy can be found in the chapter by Slack (1987) and in Goodman and Gilman's The Pharmacological Basis of Therapeutics (1980).

As indicated in these texts, some antimicrobial agents are selective in their toxicity, since they kill or inhibit the microorganism at concentrations that are tolerated by the host (i.e., the drug acts on microbial structures or biosynthetic pathways that differ from those of the host's cells). Other agents are only capable of temporarily inhibiting the growth of the microbe, which may resume growth when the inhibitor is removed. Often, the ability to kill or inhibit a microbe or parasite is a function of the agent's concentration in the body and its fluids.

Whereas these principles and the available antimicrobial drugs have been successful for the treatment of many infections, particularly bacterial infections, other infections have been resistant or relatively unresponsive to systemic chemotherapy, e.g., viral infections and certain fungal, protozoan and parasitic infections.

As used herein, “microbe” denotes virus, bacteria, Rickettsia, Mycoplasma, protozoa and fungi, while “pathogen” denotes both microbes and infectious multicellular invertebrates, e.g., helminths and the like.

Bacteria can infect host cells and “hide” from circulating systemic drugs. Even when bacterial proliferation is active and the bacteria is released from host cells, systemic agents can be insufficiently potent at levels which are tolerated by the patient. Thus, the compositions of the invention may be used in targeting therapeutics to the location that will typically be more effective in treating an infection by a pathogen.

A. Prime-Boost Vaccination Methods

When one or more compositions of the invention are administered in conjunction with or without adjuvants and/or other excipients, the antigen may be administered before, after, and/or simultaneously with the other antigenic compositions. For instance, the combination of antigens or vaccine compositions may be administered as a priming dose of antigen or vaccine composition. One or more antigen or vaccine composition may then be administered with the boost dose, including the antigen or vaccine composition used as the priming dose. Alternatively, the combination of two or more antigens or vaccine compositions may be administered with a boost dose of antigen. One or more antigen or vaccine composition may then be administered with the prime dose. A “prime dose” is the first dose of antigen administered to a subject. In the case of a subject that has an infection the prime dose may be the initial exposure of the subject to the pathogen and a combination of antigens or vaccine compositions may administered to the subject in a boost dose. A “boost dose” is a second, third, fourth, fifth, sixth, or more dose of the same or different antigen or vaccine composition administered to a subject that has already been exposed to an antigen. In some cases the prime dose may be administered with a combination of antigens or vaccine compositions such that a boost dose is not required to protect a subject at risk of infection from being infected. An antigen may be administered with one or more adjuvants or other excipients individually or in any combination. Adjuvants may be administered prior to, simultaneously with or after administration of one or more antigen(s) or vaccine compositions. It is contemplated that repeated administrations of antigen(s) as well as one or more of the components of a vaccine composition may be given alone or in combination for one or more of the administrations. Antigens need not be from a single pathogen and may be derived from one or more pathogens. The order and composition of a vaccine composition may be readily determined by using known methods in combination with the teachings described herein. Examples of the prime-boost method of vaccination can be found in U.S. Pat. No. 6,210,663, incorporated herein by reference.

In various embodiment, the time between administration of the priming dose and the boost dose may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or more days, weeks, months, or years. The vaccine compositions include, but are not limited to any of the polynucleotide, polypeptide, and binding agent compositions described herein or combination of any 1, 2, 3, 4, 5, 6, 7, 8, 9, 10, 11, 12, 13, 14, 15, 16, 17, 18, 19, 20, or more of each individual composition.

B. Passive Immunization

Methods of passively immunizing an animal or human subject against a preselected ligand or pathogen by contacting or administering to the animal or human subject a composition comprising one or more antibodies or affinity agents to an antigen(s), in particular an antigen(s) of the present invention, are contemplated by the present invention.

Immunoglobulin molecules and other affinity or binding agents are capable of binding a preselected antigen and can be efficiently and economically produced synthetically and in plant or animal cells, as well as in a variety of animals including, but not limited to horse, pig, rabbit, goat, donkey, mouse, rat, human and other organisms capable of producing natural or recombinant molecules. In various embodiments, an immunoglobulin molecule either is an IgA, IgM, secretory IgM or secretory IgA.

Secretory immunoglobulins, such as secretory IgM and secretory IgA may be resistant to proteolysis and denaturation. Contemplated environments for the administration or use of such molecules include acidic environments, protease containing environments, high temperature environments, and other harsh environments. For example, the gastrointestinal tract of an animal is a harsh environment where both proteases and acid are present. See, Kobayishi (1973). Passive immunization of an animal or human subject may be produced by contacting or administering an antibody or binding agent that recognizes an antigen of the present invention by intravascular, intramuscular, oral, intraperitoneal, mucosal, or other methods of administration. Mucosal methods of administration may include administration by the lungs, the digestive tract, the nasopharyngeal cavity, the urogenital system, and the like.

In various embodiments the antibody or binding agent, such as an immunoglobulin molecule is specific for a preselected antigen. Typically, this antigen is present on a pathogen that causes a disease. One or more antibody or binding agent may be capable of binding to a pathogen(s) and preventing or treating a disease state.

In certain embodiments, the composition comprising one or more antibody or binding agent is a therapeutic or pharmaceutically acceptable composition. The preparation of therapeutic or pharmaceutically acceptable compositions which contain polypeptides, proteins, or other molecules as active ingredients is well understood in the art and are briefly described herein.

In certain embodiments, a composition containing one or more antibody or binding agent(s) comprises a molecule that binds specifically or preferentially with a pathogen antigen. Preferentially is used herein to denote that a molecule may bind other antigens or molecules but with a much lower affinity as compared to the affinity for a preferred antigen. Pathogens may be any organism that causes a disease in another organism.

Antibodies or binding agents specific or preferential for a pathogen may be produced using standard synthetic, recombinant, or antibody production techniques. See, Antibodies: A Laboratory Manual (1988) and alternative affinity or binding agents described herein.

VI. Pharmaceutical Compositions

Compositions of the present invention comprise an effective amount of a Borrelia polynucleotide or variant thereof; an antigenic protein, polypeptide, peptide, or peptide mimetic; anti-Borrelia antibodies; and the like, which may be dissolved and/or dispersed in a pharmaceutically acceptable carrier and/or aqueous medium. Aqueous compositions of genetic immunization vectors, vaccines and such expressing any of the foregoing are also contemplated.

A. Pharmaceutical Preparations of Peptides, Nucleic Acids, and Other Active Compounds.

The Borrelia polypeptides of the invention and the nucleic acids encoding them may be delivered by any method known to those of skill in the art (see for example, “Remington's Pharmaceutical Sciences” 15th Edition).

Solutions comprising the compounds of the invention may be prepared in water suitably mixed with a surfactant, such as hydroxypropylcellulose. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions. The form should usually be sterile and must be fluid to the extent that easy syringability exists. It must be stable under the conditions of manufacture and storage and must be preserved against the contaminating action of microorganisms, such as bacteria and fungi.

For parenteral administration in an aqueous solution, for example, the solution may be suitably buffered if necessary and the liquid diluent first rendered isotonic with sufficient saline or glucose. These particular aqueous solutions are especially suitable for intravenous, intramuscular, subcutaneous, intratumoral and intraperitoneal administration. In this connection, sterile aqueous media that can be employed will be known to those of skill in the art in light of the present disclosure. In terms of using peptide therapeutics as active ingredients, the technology of U.S. Pat. Nos. 4,608,251; 4,601,903; 4,599,231; 4,599,230; 4,596,792; and/or 4,578,770, each incorporated herein by reference, may be used.

For human administration, preparations should meet sterility, pyrogenicity, general safety and purity standards as required by FDA Office of Biologics standards.

The phrase “pharmaceutically-acceptable” or “pharmacologically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared.

B. Routes of Delivery/Administration

Pharmaceutical compositions may be conventionally administered parenterally, by injection, for example subcutaneously, intradermally, or intramuscularly. However, any method for administration of a composition is applicable. These include gene gun inoculation of the DNA encoding the peptide(s), oral application on a solid physiologically acceptable base or in a physiologically acceptable dispersion, transdermal patch application, parenteral delivery, injection, or the like. The polynucleotides and polypeptides of the invention will typically be formulated for parenteral administration, such as injection via the intravenous, intramuscular, sub-cutaneous, intralesional, epidermal, transcutaneous, intraperitoneal routes. Additionally, compositions may be formulated for oral or inhaled delivery.

Injection of a nucleic acid encoding a Borrelia polypeptide may be delivered by syringe or any other method used for injection of a solution, as long as the nucleic acid encoding the Borrelia polypeptide, can pass through the particular gauge of needle required for injection. A novel needleless injection system has recently been described (U.S. Pat. No. 5,846,233) having a nozzle defining an ampule chamber for holding the solution and an energy device for pushing the solution out of the nozzle to the site of delivery. A syringe system has also been described for use in gene therapy that permits multiple injections of predetermined quantities of a solution precisely at any depth (U.S. Pat. No. 5,846,225).

C. Adjuvants

Immunogenicity can be significantly improved if the vectors or antigens are co-administered with adjuvants. Adjuvants enhance the immunogenicity of an antigen but are not necessarily immunogenic themselves. Adjuvants may act by retaining the antigen locally near the site of administration to produce a depot effect facilitating a slow, sustained release of antigen to cells of the immune system. Adjuvants can also attract cells of the immune system to an antigen depot and stimulate such cells to elicit immune responses. Adjuvants can stimulate or signal activation of cells or factors of the immune system. Exemplary adjuvants may be found in U.S. Pat. No. 6,406,705, incorporated herein by reference.

As used herein, the term “adjuvant” refers to an immunological adjuvant. By this is meant a compound that is able to enhance the immune system's response to an immunogenic substance or antigen. The term “immunogenic” refers to a substance or active ingredient which when administered to a subject, either alone or with an adjuvant, induces an immune response in the subject. The term “immune response” includes specific humoral, i.e. antibody, as well as cellular immune responses, the antibodies being serologic as well as secretory and pertaining to the subclasses IgM, IgD, IgG, IgA and IgE as well as all isotypes, allotypes, and subclasses thereof. The term is further intended to include other serum or tissue components. The cellular response includes Type-1 and Type-2 T-helper lymphocytes, cytotoxic T-cells as well as natural killer (NK) cells.

Furthermore, several other factors relating to adjuvanicity are believed to promote the immunogenicity of antigens. These include (1) rendering antigens particulate, e.g. aluminum salts, (2) polymers or polymerization of antigens, (3) slow antigen release, e.g. emulsions or micro-encapsulation, (4) bacteria and bacterial products, e.g. CFA, (5) other chemical adjuvants, e.g. poly-I:C, dextran sulphate and inulin, (6) cytokines, and (7) antigen targeting to APC.

General categories of adjuvants that may be used in conjunction with the invention includes, but is not limited to peptides, nucleic acids, cytokines, microbes (bacteria, fungi, parasites), glycoproteins, glycolipids, lipopolysaccharides, emulsions, and the like.

A combination of adjuvants may be administered simultaneously or sequentially. When adjuvants are administered simultaneously they can be administered in the same or separate formulations, and in the latter case at the same or separate sites, but are administered at the same time. The adjuvants are administered sequentially, when the administration of at least two adjuvants is temporally separated. The separation in time between the administration of the two adjuvants may be a matter of minutes or it may be longer. The separation in time is less than 14 days, and more preferably less than 7 days, and most preferably less than 1 day. The separation in time may also be with one adjuvant at prime and one at boost, or one at prime and the combination at boost, or the combination at prime and one at boost.

In some embodiments, the adjuvant is Adjumer™, Adju-Phos, Algal Glucan, Algammulin, Alhydrogel, Antigen Formulation, Avridine®, BAY R1005, Calcitriol, Calcium Phosphate Gel, Cholera holotoxin (CT), Cholera toxin B subunit (CTB), Cholera toxin A1-subunit-Protein A D-fragment fusion protein, CRL1005, Cytokine-containing Liposome, Dimethyl dioctadecylammonium bromide, Dehydroepiandrosterone; Dimyristoyl phosphatidyl choline; 1,2-dimyristoyl-sn-3-phosphatidylcholine, Dimyristoyl phosphatidylglycerol, Deoxycholic Acid Sodium Salt; Freund's Complete Adjuvant, Freund's Incomplete Adjuvant, Gamma Inulin, Gerbu Adjuvant, GM-CSF, N-acetylglucosaminyl-(β1-4)—N-acetylmuramyl-L-alanyl-D-isoglutamine, Imiquimod, ImmTher™, Interferon-γ, Interleukin-1β, Interleukin-2, Interleukin-7, Interleukin-12, ISCOM™, Iscoprep 7.0.3.™, Liposome, Loxoribine, LT-OA or LT Oral Adjuvant, MF59, MONTANIDE ISA 51, MONTANIDE ISA 720, MPL™, MTP-PE, MTP-PE Liposome, Murametide, Murapalmitine, D-Murapalmitine, NAGO, Non-Ionic Surfactant Vesicle, Pleuran, lactic acid polymer, glycolic acid polymer, Pluronic L121, Polymethyl methacrylate, PODDS™, Poly rA:Poly rU, Polysorbate 80, Protein Cochleate, QS-21, Quil-A, Rehydragel HPA, Rehydragel LV, S-28463, SAF-1, Sclavo peptide, Sendai Proteoliposome, Sendai-containing Lipid Matrix, Span 85, Specol, Squalane, Stearyl Tyrosine, Theramide™, Threonyl-MDP, Ty Particle, Walter Reed Liposome or other known adjuvants.

D. Dosage and Schedules of Administration

The dosage of the polynucleotides and/or polypeptides and dosage schedule may be varied on a subject by subject basis, taking into account, for example, factors such as the weight and age of the subject, the type of disease being treated, the severity of the disease condition, previous or concurrent therapeutic interventions, the manner of administration and the like, which can be readily determined by one of ordinary skill in the art.

Administration is in any manner compatible with the dosage formulation, and in such amount as will be therapeutically effective and/or immunogeni. The quantity to be administered depends on the subject to be treated, including, e.g., the capacity of the individual's immune system to synthesize antibodies, and the degree of protection desired. The dosage of the vaccine will depend on the route of administration and will vary according to the size of the host. Precise amounts of an active ingredient that required to be administered depend on the judgment of the practitioner.

In certain embodiments, pharmaceutical compositions may comprise, for example, at least about 0.1% of an active compound. One of the various active compounds being a Borrelia polynucleotide or polypeptide. In other embodiments, the an active compound may comprise between about 2% to about 75% of the weight of the unit, or between about 25% to about 60%, for example, and any range derivable therein. However a suitable dosage range may be, for example, of the order of several hundred micrograms active ingredient per vaccination. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per vaccination, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above. A suitable regime for initial administration and booster administrations (e.g., inoculations) are also variable, but are typified by an initial administration followed by subsequent inoculation(s) or other administration(s).

In many instances, it will be desirable to have multiple administrations of a vaccine, usually not exceeding six vaccinations, more usually not exceeding four vaccinations and preferably one or more, usually at least about three vaccinations. The vaccinations will normally be at from two to twelve week intervals, more usually from three to five week intervals. Periodic boosters at intervals of 1-5 years, usually three years, will be desirable to maintain protective levels of the antibodies.

A course of the immunization may be followed by assays for antibodies for the supernatant antigens. The assays may be performed by labeling with conventional labels, such as radionucleotides, enzymes, fluorescents, and the like. These techniques are well known and may be found in a wide variety of patents, such as U.S. Pat. Nos. 3,791,932; 4,174,384 and 3,949,064, as illustrative of these types of assays. Other immune assays can be performed and assays of protection from challenge with a nucleic acid can be performed, following immunization.

VII. Kits

The invention also relates to kits for assaying a Borrelia infection comprising, in a suitable container: (a) a pharmaceutically acceptable carrier; and (b) an antibody, or other suitable binding agent, directed against a Borrelia antigen.

Therapeutic kits of the present invention are kits comprising a Borrelia (e.g., B. burgdorferi or B. afzelii a) polynucleotide or polypeptide or an antibody to the polypeptide. Such kits will generally contain, in a suitable container, a pharmaceutically acceptable formulation of an Borrelia polynucleotide or polypeptide, or an antibody to the polypeptide, or vector expressing any of the foregoing in a pharmaceutically acceptable formulation. The kit may have a single container, and/or it may have a distinct container for each compound.

When the components of the kit are provided in one and/or more liquid solutions, the liquid solution is an aqueous solution, with a sterile aqueous solution being particularly preferred. The Borrelia polynucleotide or polypeptide, or antibody compositions may also be formulated into a syringeable composition. In which case, the container may itself be a syringe, pipette, and/or other such like apparatus, from which the formulation may be applied to an infected area of the body, injected into an animal, and/or even applied to and/or mixed with the other components of the kit.

However, the components of the kit may be provided as dried powder(s). When reagents and/or components are provided as a dry powder, the powder can be reconstituted by the addition of a suitable solvent. It is envisioned that the solvent may also be provided in another container.

The container will generally include at least one vial, test tube, flask, bottle, syringe and/or other container, into which the Borrelia polynucleotide or polypeptide, or antibody formulation are placed, preferably, suitably allocated. The kits may also comprise a second container for containing a sterile, pharmaceutically acceptable buffer and/or other diluent.

The kits of the present invention will also typically include a means for containing the vials in close confinement for commercial sale, such as, e.g., injection and/or blow-molded plastic containers into which the desired vials are retained.

Irrespective of the number and/or type of containers, the kits of the invention may also comprise, and/or be packaged with, an instrument for assisting with the injection/administration and/or placement of the ultimate Borrelia polynucleotide or polypeptide, or an antibody to the polypeptide within the body of an animal. Such an instrument may be a syringe, pipette, forceps, and/or any such medically approved delivery vehicle.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Construction of a Borrelia burgdorferi Expression Library.

Borrelia burgdorferi (BBU) stock isolate 910255, obtained from Dr. Steven Nickell, was originally isolated from a wild mouse. The bacteria were grown at 33° C. under anaerobic conditions, and genomic DNA was isolated by published procedures (Hinnebusch and Barbour, 1992). The library production protocol was similar to that previously described to generate HIV and SIV random expression libraries (Sykes and Johnston, 1999 and Sykes et al., 2002). Briefly, Borrelia DNA was physically sheared with a nebulizer (Glas-Col, Terre Haute, Iowa.), and stripped ends were mended with Klenow and T4 polymerase Fragments from 300 to 800 base pairs (bp) were size-selected by electrophoresis through a 1.5% agarose TRIS-borate gel, then excised and electroeluted. To convert the blunt DNA to sticky ended fragments for cloning, Bg/II adaptors were designed. Two oligonucleotides (15-mer: GATCTGGATCCAGGC (SEQ ID NO:140), 11-mer: GCCTGGATCCA (SEQ ID NO:141)) were annealed and phosphorylated. The genomic fragments and adaptors were blunt-end ligated to generate Bg/II compatible inserts. The products were purified on a DNA-binding filter unit (Qiaquick, Qiagen, Valencia, Calif.), and cloned into the dephosphorylated Bg/II site of immunization vector pCMVitPA (Sykes et al., 2002). The cloning site positions inserts to be expressed by the upstream cytomegalovirus immediate early gene promoter, embedded with a chimeric intron to stabilize transcripts and increase gene expression (Manthorpe et al., 1993). A translational start and secretory leader sequence is encoded by 83 nucleotides from the tissue-plasminogen activator (tPA) gene. Inserts are expressed as fusions to this leader peptide, corresponding to the first 23 residues of the unprocessed secretory protein. The human growth hormone polyadenylation sequence is provided downstream of the Bg/II site to provide efficient termination. The ligation products were used to transform DH5alpha E. coli.

A test transformation of a small, defined portion of the ligation was performed to analyze the library. Colony counts were used to determine transformation efficiency so that subsequent platings could be calculated. PCR-amplification of the plasmid inserts directly from the colonies was used to determine insert size and cloning efficiency. Nearly 100% of the plasmids carried inserts, averaging 560 base pairs in length. More than 24 inserts were sequenced to verify Borrelia identity. The full ligation reaction was subsequently transformed and plated at a calculated subconfluency onto 40 bioassay trays (20 cm×20 cm) containing LB agar and ampicillin. The bioassay trays were incubated at 37° C. overnight to obtain between 1000 and 1500 colonies per tray. Each tray would represent one sublibrary group for ELI-screening. The original library transformants were lifted onto nitrocellulose filters, which were prepared by impregnated them with LB and 10% glycerol. The filters were placed at −80° C. for storage while the original colonies on the bioassay trays were further grown then replica-plated onto additional agar plates for bacterial amplification. Plates were incubated at 37° C. overnight and then bacterial cells were harvested. The mixed-plasmid DNA samples that corresponded to each of 40 expression library pools were purified with endotoxin-free DNA-binding column kits (Qiagen tip-500). The DNA quality and integrity of pool complexities were verified by spectrophotometry, enzyme digestion, and gel electrophoresis.

Using the information gathered about the library, the representation of the 1.4 megabase (Mb) Borrelia burgdorferi genome was statistically calculated. Each clone carries a randomly inserted genomic fragment from either the 911 kilobase (kb) Borrelia chromosome, or one of the extrachromosomal elements. These 12 linear and 9 circular episomal DNAs total 533 kb (Fraser et al., 1997 and Casjens et al., 2000). The proportion of chromosomal- and extrachromosomal-derived clones matched that of the genome. There is an average of 1250 clones in each of the 40 sub-library pools with 99% carrying Borrelia DNA. Each of these fragments averages 560 bp, and holds a 1 in 6 probability of having been inserted in the proper orientation and frame relative to the expression plasmid elements. Given these parameters, each pool should properly express the average equivalent of 0.08 of the genome ((1250×0.99)×560×(⅙)/1,444,000=0.08 expression equivalents). Together, the 40 sub-libraries represent 3.2 genome expression equivalents.

### Example 2

### Borrelia burgdorferi Expression Library Immunization and Challenge, Round 1

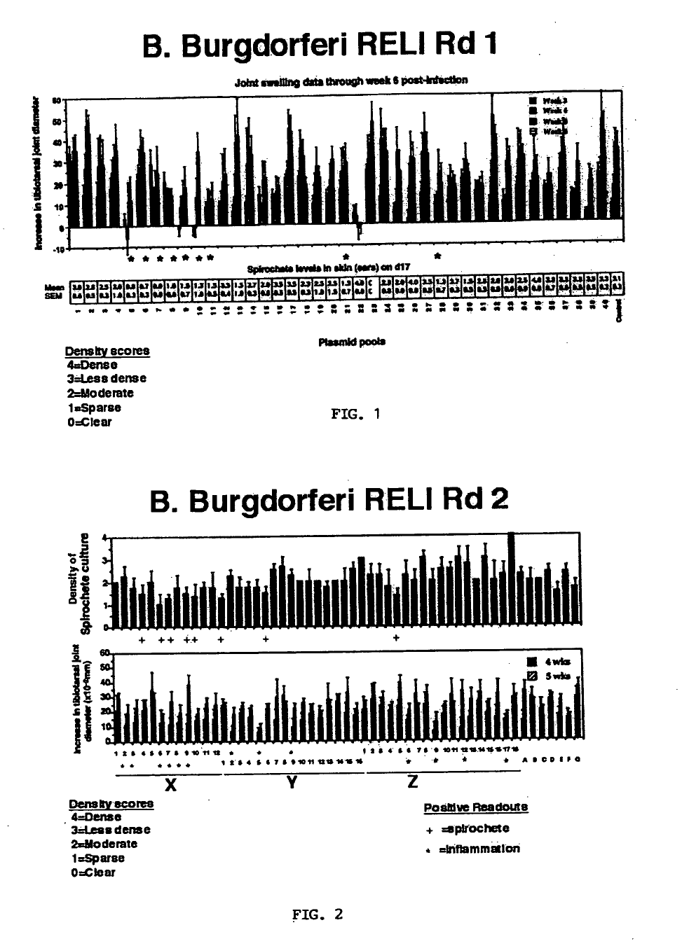

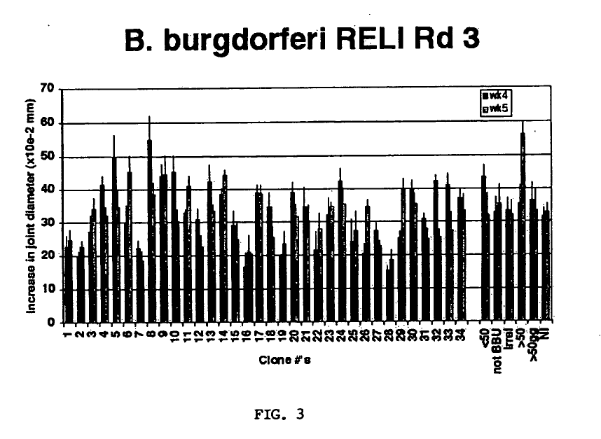

The 40 sub-library mixed-DNA samples were combined with a plasmid encoding murine granulocyte-macrophage colony-stimulating factor (GM-CSF) at 1/10th library DNA dose in phosphate buffered saline (PBS). These inocula were intramuscularly (i.m.) injected into 40 groups (4 mice per group) of 6-week old C3H/NeH mice. Each mouse received each 50 μg of Borrelia library and 5 μg of pCMViGMCSF (Smooker et al., 2000, and Xiang and Ertl, 1995), distributed between four sites, left and right quadricep and tibialis anterior muscles. The mice were administered boosts with the identical inocula at weeks 8 and 12. The vaccinees were challenged subcutaneously with 100 μl of a B. burgdorferi inoculum (10⁵ organisms) 2 weeks after the last immunization, and then monitored for infection and disease based on two readouts over the course of 6 weeks. To assess infection, ear skin samples were removed 17 days post-challenge to assess spirochete densities. After 6 days in growth media, spirochetes were counted under a microscope and scored on a 5-point scale. 4=dense,full field (10⁵ organisms) 3=less dense>200/quarter field (10⁴ organisms), 2=moderate 50/quarter field (10³ organisms), 1=sparse 10/field (10² organisms), 0=clear. As a parameter of disease severity, the diameter of each tibiotarsel joint was measured in duplicate at weeks 3, 4, 5, 6 post-challenge to determine swelling. Inflammation scores were assigned by subtracting the average joint diameter of uninfected mice from the diameter of each of the experimental mice, at each time point. The combined readout results of the spirochete and inflammation data are presented in FIG. 1. Sub-library inocula were selected for further partitioning based on their conferring both reduced disease and reduced infection levels to immunized mice. An exception was made for sub-library 22. It was not carried forward despite displaying very little inflammation, because spirochete levels were scored as 4 (dense). Nine sub-libraries (#5, #6, #7, #8, #9, #10, #11 #21, #28) comprised of 10,272 colonies were selected to for arraying and retesting in round 2.

### Example 3

### Array Analysis of the Expression Library, Round 2.

The components for the second round of sib testing were retrieved from the nine nitrocellulose filter-stocks that corresponded to the nine positively scored sublibrary inocula of round 1. The filters were replica-plated onto fresh bioassay trays and colonies were regrown. Single, original transformants were now available to be transferred into a robotic format. Toothpicks were used to inoculate individual microtiter-plate cultures containing HYT freezing media (1.6% Bacto-tryptone, 1.0% Bacto-yeast extract, 85.5 mM NaCl, 36 mM K₂HPO₄, 13.2 mM KH₂PO₄, 1.7 mM Sodium citrate, 0.4 mM MgSO₄, 6.8 mM ammonium sulfate, 4.4% wt/vol glycerol) supplemented with 75 μg/mL ampicillin, and were grown overnight at 37° C. Growth and storage of the libraries as mini-cultures served to permanently maintain the original library complexity. Using a stamping tool, 20×20 cm LB-carbenicillin/lincomyocin agar plates were inoculated with a set of the bacterial transformants that would define the new groups of plasmid members for round-2 testing. The group compositions were determined by positioning each transformant into a virtual cubic matrix, then combining the bacteria according to the virtual three-dimensional axes. By this pooling method, each transformant was located in three unique pools, corresponding to once in each of three axes. The objective was to map our protection assay data onto this grid such that a matrix analyses of the axes intersections would efficiently identified single transformants correlated with protection. The round-2 grid was built with 12X -axes, 16 Y-axes, and 18 Z-axes, which creates 3456 loci (12×16×18). All 10,272 round 1 clones were assigned positions within the grid by placing 3 clones per locus. The 46 groups (12+16+18) contained plasmid complexities of 576 (Z), 672 (Y), or 864 (X). Culture-stamped bioassay trays were incubated at 37° C. overnight and then E. coli were harvested. The 46 mixed-plasmid library pools were purified with endotoxin-free Qiagen tip-500 column kits. The DNA quality and the integrity of pool complexities were verified by spectrophotometry, enzyme digestion, and gel electrophoresis. Like round 1, 50 μg of the mixed-plasmid inocula was injected intramuscularly. In this round and in subsequent ones, another 1 μg of the same DNA was shot into each mouse ear with a gene gun (Helios, BioRad, Inc.). In addition to the newly arrayed round-2 pools, several round-1 sub-library pools (#5, #7, #21, #22, #28), and two sub-libraries constructed from non-Borrelia bacterial DNA were included. Boosts were administered at weeks 6 and 12 and mice were subcutaneously injected with B. burgdorferi spirochetes (5×10⁵ organisms in 100 μl) 4 weeks after the last immunization. Disease was monitored as described for round 1. To measure infection levels, ear tissue was removed on day 21 post-challenge, spirochetes were grown in media 6 days, and samples were pipetted onto slides for microscope analysis. Density was qualitatively scored from 0 to 4. To measure the disease phenotype of inflammation, tibiotarsel joint diameters were measured in duplicate for each leg at 4 and 5-week timepoints post-challenge and calculated as described for round 1. The two sets of readout results presented in FIG. 2 did not overlap, therefore the spirochete densities and the joint swelling results were considered as separate data sets. The groups that scored positively by spirochete densities (X4, X6, X7, X9, X10, Y1, Y6, Z4) were used to designate one set of matrix intersections and the joint swelling positives (X2, X3, X6, X7, X8, X9, Y2, Y5, Y9, Z6, Z9, Z12, Z17) were used to generate an independent set.

From the spirochete data matrix 10 intersections were identified, and from the swelling data matrix 72 intersections were pinpointed, for a total of 82 matrix designations. Each matrix loci contained 3 transformants, therefore 246 microtiter-well bacterial mini-stocks were designated. Each of these stocks were retrieved, separated into individual transformants. Plasmid clones were purified and then analyzed by sequencing. Half of the Borrelia DNA fragments were derived from the chromosome and half from one of the large episomal plasmids. This distribution approximates that of the total 1.4 megabases of Borrelia DNA. If open-reading-frames (ORFs) are defined as sequences that encode greater than 50 amino acids, 74 clones were identified. However only 34 of these correspond to properly expressed, B. burgdorferi genes according to the annotated GenBank database. Within the group of 34 gene-fragment clones, 31 were intersections of positive groups based on inflammation data and only 3 (clone #'s 25, 27, 29) were indicated from intersections of positive groups based on spirochete data. The identities of the B. burgdorferi genes from which the 34 gene fragments are derived are listed in Table 2. The nucleotide and amino acid sequences of both the gene fragments contained on the library clones and the corresponding full gene sequences are provided in the sequence listing.

TABLE 2
Gene Derivations of RELI-Identified ORFs as Borrelia
Vaccine Candidates

| Clone | | B. burgdorferi Full length coding |
|---|---|---|
| number | Fragment | region. |
| Clone 1. | plasmid lp56. (It exists as an | Coding region BBR01 from plasmid |
| | incomplete ORF on this plasmid | cp32-4. |
| | lp56; a complete ORF resides on | SEQ ID NO: 3 and SEQ ID NO: 4 |
| | plasmid cp32-4. | |
| | SEQ ID NO: 1 and SEQ ID NO: 2 | |
| Clone 2. | chromosome | Coding region BB0144 (proX gene). |
| | SEQ ID NO: 5 and SEQ ID NO: 6 | Translated full-length predicted coding |
| | | region BB0144 glycine betaine, L-proline |
| | | ABC transporter, glycine/betaine/L- |
| | | proline-binding protein |
| | | SEQ ID NO: 7 AND SEQ ID NO: 8 |
| Clone 3. | SEQ ID NO: 9 and SEQ ID NO: 10 | Predicted coding region BB0656. |
| | | Translated full-length predicted coding |
| | | region BB0656 oxygen-independent |
| | | coproporphyrinogen III oxidase |
| | | SEQ ID NO: 11 AND SEQ ID NO: 12 |
| Clone 4. | plasmid lp25 | Predicted coding region BBE02. |
| | SEQ ID NO: 13 and SEQ ID NO: 14 | Translated full-length predicted coding |
| | | region BBE02. |
| | | SEQ ID NO: 15 AND SEQ ID NO: 16 |
| Clone 5. | plasmid cp32-7 | Predicted coding region BBO11. |
| | SEQ ID NO: 17 AND SEQ ID NO: 18 | Translated full-length predicted coding |
| | | BBO11. |
| | | SEQ ID NO: 19 AND SEQ ID NO: 20 |
| Clone 6. | plasmid lp28-1(stop codon AA | Similar to predicted coding region |
| | doesn't match published sequence but | BBF13. |
| | nucleotide does) | Translated full-length predicted coding |
| | SEQ ID NO: 21 AND SEQ ID NO: 22 | region BBF13 |
| | | SEQ ID NO: 23 AND SEQ ID NO: 24 |
| Clone 7. | Chromosome | Predicted coding region BB0508, GTP |
| | SEQ ID NO: 25 AND SEQ ID NO: 26 | binding protein. Translated full-length |
| | | predicted coding region BB0508 |
| | | SEQ ID NO: 27 AND SEQ ID NO: 28 |
| Clone 8. | Two fused inserts: one from gene | Predicted coding region BB0540 |
| | BB0540 and the second from | translation factor G (fus-1) |
| | BB0176. | Translated full-length predicted coding |
| | SEQ ID NO: 29 AND SEQ ID | region BB0540 translation elongation |
| | NO: 30 | factor G (fus-1) |
| | | SEQ ID NO: 31 AND SEQ ID NO: 32 |
| Clone 9. | chromosome | Predicted coding region BB0056 |
| | SEQ ID NO: 33 AND SEQ ID NO: 34 | phosphoglycerate kinase (pgk gene) |
| | | translated full-length predicted coding |
| | | region translated BB0056 |
| | | phosphoglycerate kinase |
| | | SEQ ID NO: 35 AND SEQ ID NO: 36 |
| Clone 10. | plasmid cp32-7 | Predicted coding region BBO29 |
| | SEQ ID NO: 37 AND SEQ ID NO: 38 | translated full-length predicted coding |
| | | region BBO29 |
| | | SEQ ID NO: 39 AND SEQ ID NO: 40 |
| Clone 11. | plasmid lp38 | Predicted coding region BBJ12 |
| | SEQ ID NO: ID NO: 41 AND SEQ | Translated full-length predicted coding |
| | ID NO: 42 | region BBJ12 |
| | | SEQ ID NO: 43 AND SEQ ID NO: 44 |
| Clone 12. | chromosome | Predicted coding region BB0342 |
| | SEQ ID NO: 45 AND SEQ ID NO: 46 | translated full-length predicted coding |
| | | region BB0342 glu-tRNA |
| | | amidotransferase, subunit A (gluA) |
| | | SEQ ID NO: 47 AND SEQ ID NO: 48 |
| Clone 13. | plasmid lp28-2 | Predicted coding region BBG24 |
| | SEQ ID NO: 49 AND SEQ ID NO: 50 | translated full-length predicted coding |
| | | region BBG24 |
| | | SEQ ID NO: 51 AND SEQ ID NO: 52 |
| Clone 14. | chromosome | Predicted coding region BB0072 |
| | SEQ ID NO: 53 AND SEQ ID NO: 54 | translated full-length predicted coding |
| | | region BB072 |
| | | SEQ ID NO: 55 AND SEQ ID NO: 56 |
| Clone 15. | chromosome | Predicted coding region BB0623 |
| | SEQ ID NO: 57 AND SEQ ID NO: 58 | translated full-length predicted coding |
| | | region BB0623 transcription-repair |
| | | coupling factor (mfd) |
| | | SEQ ID NO: 59 AND SEQ ID NO: 60 |
| Clone 16. | plasmid cp32-6 | Predicted coding region BBM11 |
| | SEQ ID NO: 61 AND SEQ ID NO: 62 | Translated full-length predicted coding |
| | | region BBM11 |
| | | SEQ ID NO: 63 AND SEQ ID NO: 64 |
| Clone 17. | chromosome | Predictedcoding region BB0211 |
| | SEQ ID NO: 65 AND SEQ ID NO: 66 | Translated full-length predicted coding |
| | | region BB0211 DNA mismatch repair |
| | | protein (mutL) |
| | | SEQ ID NO: 67 AND SEQ ID NO: 68 |
| Clone 18. | plasmid lp28-1 | Predicted coding region BBF05 |
| | SEQ ID NO: 69 AND SEQ ID NO: 70 | Translated full-length predicted coding |
| | | region BBF05 |
| | | SEQ ID NO: 71 AND SEQ ID NO: 72 |
| Clone 19. | plasmid cp32-6 | Predicted coding region BBM10 |
| | SEQ ID NO: 73 AND SEQ ID | Translated full-length coding region |
| | NO: 74 | BBM10 |
| | | SEQ ID NO: 75 AND SEQ ID NO: 76 |
| Clone 20. | plasmid cp32-3 | Predicted coding region BBS36 |
| | SEQ ID NO: 77 AND SEQ ID NO: 78 | Translated full-length predicted coding |
| | | region BBS36 |
| | | SEQ ID NO: 79 AND SEQ ID NO: 80 |
| Clone 21. | chromosome | Predicted coding region BB0072 |
| | SEQ ID NO: 81 AND SEQ ID NO: 82 | Translated full-length predicted coding |
| | | region BB0072 |
| | | SEQ ID NO: 83 AND SEQ ID NO: 84 |
| Clone 22. | chromosome (includes extra AA at | Predicted coding region BB0241 glycerol |
| | N-terminus) | kinase (glpK) Translated full-length |
| | SEQ ID NO: 85 AND SEQ ID NO: 86 | predicted coding region BB0241 glycerol |
| | | kinase (glpK) |
| | | SEQ ID NO: 87 AND SEQ ID NO: 88 |
| Clone 23. | chromosome | Predicted coding region BB0351 |
| | SEQ ID NO: 89 AND SEQ ID NO: 90 | Translated full-length predicted coding |
| | | region BB0351 |
| | | SEQ ID NO: 91 AND SEQ ID NO: 92 |
| Clone 24. | plasmid lp54 | Predicted coding region BBA04, antigen |
| | SEQ ID NO: 93 AND SEQ ID NO: 94 | S2. Translated full-length predicted |
| | | coding region BBA04 |
| | | SEQ ID NO: 95 AND SEQ ID NO: 96 |
| Clone 25. | chromosome | Predicted coding region BB0515 |
| | This insert has a ˜20 bp additional | thioredoxin reductase (trxB) Translated |
| | sequence added to the 3′ end | full-length predicted coding region |
| | SEQ ID NO: 97 AND SEQ ID NO: 98 | BB0515 thioredoxin reductase (trxB) |
| | | SEQ ID NO: 99 AND SEQ ID NO: 100 |
| Clone 26. | plasmid cp26 | Predicted coding region BBB14 |
| | (includes AA before N-terminus) | Translated full-length predicted coding |
| | SEQ ID NO: 101 AND SEQ ID | region BBB14 |
| | NO: 102 | SEQ ID NO: 103 AND SEQ ID NO: 104 |
| Clone 27. | chromosome | Predicted coding region BB0230 |
| | SEQ ID NO: 105 AND SEQ ID | Translated full-length predicted coding |
| | NO: 106 | region BB0230 transcription termination |
| | | factor Rho (rho) |
| | | SEQ ID NO: 107 AND SEQ ID NO: 108 |
| Clone 28. | plasmid lp28-1 | Putative vls recombination cassette Vls8 |
| | SEQ ID NO: 109 AND SEQ ID | Translated putative vls recombination |
| | NO: 110 | cassette Vls8 and Vls9 |
| | | Putative vls recombination cassette Vls9 |
| | | Translated putative vls recombination |
| | | cassette Vls9 |
| | | Putative vls recombination cassettes Vls2-Vls16b |
| | | (vls) |
| | | VlsE1, variable major protein-like |
| | | sequence |
| | | Translated full-length vlsE1, variable |
| | | major protein-like sequence |
| | | SEQ ID NO: 111 AND SEQ ID NO: 112 |
| | | SEQ ID NO: 113 AND SEQ ID NO: 114 |
| | | SEQ ID NO: 115 AND SEQ ID NO: 116 |
| | | SEQ ID NO: 117 |
| Clone 29. | comprises two joined fragments: one | Putative coding region gpsA Translated |
| | from gene BB0368 and one from | full-length predicted coding region |
| | BB0333 | BB0368 NAD_Gly3P_dh, NAD- |
| | SEQ ID NO: 118 AND SEQ ID | dependent glycerol-3-phosphate |
| | NO: 119 | dehydrogenase (gpsA) |
| | | SEQ ID NO: 120 AND SEQ ID NO: 121 |
| Clone 30. | chromosome | Fortuitous ORF; it does not code for an |
| | SEQ ID NO: 122 AND SEQ ID | in-frame gene. |
| | NO: 123 | |
| Clone 31. | chromosome | Predicted coding region BB0451 |
| | SEQ ID NO: 124 AND SEQ ID | Translated full-length predicted coding |
| | NO: 125 | region BB0451 chromate transport |
| | | protein, putative |
| | | SEQ ID NO: 126 AND SEQ ID NO: 127 |
| Clone 32. | plasmid lp5 | Predicted coding region BBT01 |
| | SEQ ID NO: 128 AND SEQ ID | Translated full-length predicted coding |
| | NO: 129 | region BBT01 |
| | | SEQ ID NO: 130 AND SEQ ID NO: 131 |
| Clone 33. | chromosome | Predicted coding region BB0133 |
| | SEQ ID NO: 132 AND SEQ ID | Translated full-length predicted coding |
| | NO: 133 | BB0133 |
| | | SEQ ID NO: 134 AND SEQ ID NO: 135 |
| Clone 34. | chromosome | Predicted coding region BB0043 |
| | SEQ ID NO: 136 AND SEQ ID | Translated full-length predicted coding |
| | NO: 137 | region BB0043 |
| | | SEQ ID NO: 138 AND SEQ ID NO: 139 |

### Example 4

### Immunization with Single Clone, Matrix-Defined Candidates, Round 3

The microtiter-stock bacterial cultures carrying each of the 34 library clones indicated above were grown in liquid culture by standard methods and the plasmids were purified with Qiagen endotoxin-free kits. The library plasmid was diluted into empty plasmid DNA (PUC118) for these single clone injections, to partially offset the increase in antigen dose due to the decrease in pool complexity. Adding filler DNA allowed for the maintainance of the total amount of DNA delivered relative to previous rounds, decreasing the possibility of overdosing. The round-3 DNA samples for i.m. vaccination of mice were prepared by mixing 1 μg of each library clone with 49 μg pUC 1 18 as “filler DNA”. Microprojectiles were prepared for gene gun delivery of inocula with samples 200 ng of the library clone and 800 ng pUC 118 per earshot. The animals were boosted with the same inocula at weeks 6 and 12. Four weeks following the last boost, each test animal was subcutaneously challenged with 105 Borrelia spirochetes.

Readout analysis was focused on the joint diameter data, since joint swelling is a direct and quantitative measure of Lyme disease, whereas spirochete counting is indirect and often technically variable. Large joint diameters of the mice were measured at weeks 2, 3, 4, and 5 as described above. To assess disease, the changes in tibiotarsal joint diameter relative to that of baseline mice were calculated. Time course analyses of this mouse model of Lyme disease have shown that inflammation peaks between four and five weeks post-exposure (Potter et al., 2000). The results at these time points are shown in FIG. 3. At 4-weeks PI, animal immunized with four clones (2, 16, 19, and 28) displayed reduced inflammation relative to the uninfected group at a 95% confidence level (p<0.05). A total of seven Borrelia gene fragments conferred reduced swelling at an 85% confidence interval (p<0.15) (clones #1, 2, 7, 16, 19, 26, and 28). At the 5-week time point, four groups, those immunized with clones #2, 7, 27, and 28, displayed reduced joint swelling data relative to the uninfected mice within a 95% confidence interval. A total of ten groups, those immunized with clones #1, 2, 7, 12, 16, 19, 27, 28, 31, 32, conferred ameliorated inflammation at an 85% interval.

Combined analysis of the swelling-measurement time points indicate the following results. Two fragments (clones #2 and 28) were protective against Lyme disease-associated joint-inflammation at both critical time points PI (4 and 5 weeks) with a 95% confidence limit. Six fragments (clones # 2, 7, 16, 19, 27, 28) conferred mice significant reductions in the disease symptom at one or both of the time points PI within a 95% confidence limit. Typically, new antigens need to be tested in other host species. Since the protective capacities of the candidates may quantitatively differ in these different genetic backgrounds, consideration of a broader confidence limit of 85% is believed to be appropriate. In addition, optimizing delivery, composition, adjuvant and targeting may improve the strength of any particular candidate. Six fragments (clones # 1, 2, 7, 16, 19, and 28) reduced joint-swelling measured at both time points, and a total of eleven fragments (clones # 1, 2, 7, 12, 16, 19, 26, 27, 28, 31, 32) ameliorated disease measured at one or both of the time points PI, within a 85% confidence limit.

### Example 5

### Analysis of ELI-Identified Vaccine Candidates.

The eleven Borrelia fragments identified by ELI as carrying the capacity to protect against mouse tibiotarsal joint swelling are described in Table 3. The size of the plasmid insert, the size of the coding region within that fragment, and the size of the corresponding full-length gene are given. None of the vaccine candidates have been previously demonstrated to confer protection against disease. It is not surprising that OspA or OspC was not identified in the ELI screen since we scored positives based on the disease phenotype of joint swelling. Both of the previously used Osp antigens have been implicated in leading to inflammation in model animals (Poland and Jacobson, 2001). Mice immunized with clones 2 and 28 displayed post challenge reductions in joint swelling that were statistically significant at both 4 weeks (p=0.02 and 0.008, respectively) and 5 weeks (p=0.046 and 0.032, respectively). These clones encode a portion of the proX gene, and a portion of the vls 8 and 9 gene cassettes. The proX encodes a glycine-betaine, L-proline ABC transporter, glycine/betaine/L-proline-binding protein. As a class, the ABC genes encode the large transmembrane proteins. They are found in both bacteria and eukaryotes, and function by binding ATP to drive non-diffusable molecules (such as proline) into the cell (Dean et al., 2001). The vls8 and vls9 sequences are silent gene cassettes that can recombine into the expressed VlsE1 locus. The center of the VlsE1 recombination cassette region has 92% sequence identity with 15 contiguous upstream regions of approximately 500 bp each, located on the linear plasmid lp28-1 (Iyer et al., 2000, and Zhang et al., 1997). The VlsE gene encodes a surface-exposed lipoprotein that is found in high-infectivity but not low-infectivity strains of B. burgdorferi. The locus was originally identified by its correlation with infectivity (U.S. Pat. No. 6,437,116 B1). VlsE undergoes antigenic variation through segmental recombination with the silent cassettes (Zhang et al., 1997). The VMP-like sequence (vls) locus resembles a previously characterized genetic variation system of B. hermsii that expresses the variable major proteins (VMPs). By homology, VlsE of B. burgdorferi encodes a large VMP-lke protein (Vlp). OspC is a member of the small VMP-like proteins (Vsp).

Homologues of the B. burgdorferi vaccine candidates identified in this screen are envisioned to be protective in some related Borrelia species such as B. afzelii, B. garinii, or B. hermsii. These homologous may have utility as antigens against these borrelia diseases. Unfortunately the genomes of these species are not sequenced. However the gene product encoded by the fragment on clone #1 (BBR01) displays 76% identity to a B. hermsii gene available in GenBank. The B. hermsii homolog of B. burgdorferi BBR01 may carry protective capacity against B. hermsii. Additionally, vaccination with genes from one borrelia species might heterologously protect against exposure to a different borrelia species.

TABLE 3
RELI protection assays identify protective
Borrelia vaccine candidates.

| | | Library | Coding | Full length |
|---|---|---|---|---|
| BBU clone No. | Gene Name | Insert | Fragment | Gene |
| #1 | BBR01 | 820 | 366 | 1224 |
| #2 | ProX | 275 | 276 | 873 |
| #7 | BB0508 | 509 | 204 | 1302 |
| #12 | gluA | 386 | 386 | 1491 |
| #16 | BBM11 | 663 | 483 | 1113 |
| #19 | BBM10 | 965 | 234 | 570 |
| #26 | BBB14 | 225 | 222 | 498 |
| #27 | rho | 896 | 897 | 1548 |
| #28 | vls8-vls9 | 318 | 318 | 1071(vlsE) |
| #31 | BB0451 | 727 | 90 | 534 |
| #32 | BBT01 | 213 | 210 | 444 |

### Example 6

### Creation and Testing of Vaccines Using Combinations of the ELI-Identified Borrelia Nucleic Acid and Amino Acid Sequences.

The Borrelia sequences and antigens claimed as protective candidates could be developed into vaccines for Borreliosis in humans and animals in the following manner. The genetic-antigens, genetic-antigen fragments, protein antigens or protein antigen fragments may be combined with one another. These might be delivered as single or sequential inocula. These may be delivered by a combination of modalities, such as genetic, protein, or live-vectored. Alternatively, the functional or sequence homologues of the identified antigen candidates from multiple Borrelia species might be combined to produce broader protection against multiple species in one vaccine.

### Example 7

### Creation and Testing of Vaccines Against Other Borrelia Species Using B. burgdorferi Nucleic Acid and Amino Acid Sequences

The Borrelia sequences and antigens disclosed in this application are envisioned to be used in vaccines for Borrelia diseases in humans and commercially important animals. However, these sequences may be used to create vaccines for other species as well, including other species of the Borrelia genus. For example, one may use the information gained concerning Borrelia to identify a sequence in another bacterial pathogen that had substantial homology to the Borrelia sequences. In many cases, this homology would be expected to be more than 30% amino acid sequence identity or similarity and could be for only part of a protein, e.g., 30 amino acids, in the other species. The gene encoding such identity/similarity may be isolated and tested as a vaccine candidate in the appropriate model system either as a protein or nucleic acid. Alternatively, the Borrelia homologs may be tested directly in an animal species of interest since having so few genes to screen (10 or less) and given that the genes had been demonstrated to be protective in another species the probability of success would be high. Alternatively, proteins or peptides corresponding to the homologs to the Borrelia genes may be used to assay in animals or humans for immune responses in people or animals infected with the relevant pathogen. If such immune responses are detected, particularly if they correlated with protection, then the genes, proteins or peptides corresponding to the homologs may be tested directly in animals or humans as vaccines.

### Example 8

### Creation and Testing of Commercial Vaccines Using Borrelia Nucleic Acid and Amino Acid Sequences

The genes identified and claimed as vaccine candidates can be developed into commercial vaccines in the following manner. The genes identified can be converted to optimize mammalian expression by changing the codons. This is a straightforward procedure, which can be easily done by one of skill in the art. Alternatively, a protective gene vaccine might be sequence-optimized by shuffling homologs from other borrelia (Stemmer et al., 1995). This might increase efficacy against spirochete exposure and/or provide a vaccine that protects against multiple Borrelia. The genes can then be tested in the relevant host, for example, humans, for the relevant protection. Genetic immunization affords a simple method to test vaccine candidate for efficacy and this form of delivery has been used in a wide variety of animals including humans. Alternatively, the genes may be transferred to another vector, for example, a vaccinia vector, to be tested in the relevant host in this form. Alternatively, the corresponding protein, with or without adjuvants may be tested. These tests may be done on a relatively small number of animals. Once conducted, a decision can be made as to how many of the protective antigens to include in a larger test. Only a subset may be chosen based on the economics of production. A large field trial may be conducted using the formulation arrived at. Based on the results of the field trial, possibly done more than once at different locations, a commercial vaccine would go into production.

### Example 10

### Creation and Testing of Vaccines Against Other Pathogens Using Borrelia Nucleic Acid and Amino Acid Sequences

Since B. burgdorferi has a similar biology to other Borrelia the inventors take advantage of the screening already accomplished on the Borrelia genome to test other species for homologs corresponding to the ones from B. burgdorferi as vaccine candidates. Those of ordinary skill may expect that, as one moved evolutionarily away from B. burgdorferi, the likelihood that the homologs would protect would presumably decline. However, researchers would be likely to test the homologs identified from even disparate species for protective ability in regard to relevant diseases, as this could reduce the search of a genome for vaccine candidates ˜200-1,000 fold. Once the homologs have been identified and isolated, they may be tested in the appropriate animal model system for efficacy as a vaccine. For example, other Borrelia homologs as genes or proteins can be tested in a mouse model of borreliosis.

One of ordinary skill has access to borrelia sequences disclosed in this specification, or to additional sequences determined to be protective using any of the methods disclosed in this specification, it is easy to run a computer-based search of relevant genetic databases in order to determine homologous sequences in other pathogens. For example, these searches can be run using the BLAST program in GenBank or other databases.

Once a sequence homologous to a protective sequence is determined, it is possible to obtain the homologous sequence using any of a number of methods known to those of skill. For example, it is easy to PCR amplify the pathogen homolog genes from genomic DNA and clone the genes into an appropriate genetic immunization vector, such as those used for ELI. These homolog genes can then be tested in an animal model appropriate for the pathogen for which protection is sought, to determine whether homologs of borrelia genes will protect a host from challenge with that pathogen.

Of course, it is possible for one of ordinary skill to use the borrelia genes that are disclosed as protective herein, or determined to be protective using the methods disclosed herein, to obtain protective sequences from a first non-borrelia organism, then to use the protective sequences from the non- borrelia organism to search for homologous sequences in a second non- borrelia or borrelia organism. So long as a protective borrelia sequence is used as the starting point for determining at least one homology in such a chain of searches and testing, such methods are within the scope of this invention.

### Example 11

### Creation and Testing of Diagnostic or Drug Targets Using Borrelia Nucleic Acid and Amino Acid Sequences

The genes identified and claimed as vaccine candidates can be developed into commercial diagnostic candidates in the following manner. It is envisioned that antigens useful in raising protective immune responses may also engender rapidly detectable host responses that could be useful for identification of pathogen exposure or early-stage infection. In addition these antigens may designate key pathogen targets for developing drug-based inhibition or therapies of infection or disease.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 3,447,851
U.S. Pat. No. 3,791,932
U.S. Pat. No. 3,949,064
U.S. Pat. No. 4,148,876
U.S. Pat. No. 4,174,384
U.S. Pat. No. 4,179,337
U.S. Pat. No. 4,406,885
U.S. Pat. No. 4,444,887
U.S. Pat. No. 4,512,972
U.S. Pat. No. 4,554,101
U.S. Pat. No. 4,578,770
U.S. Pat. No. 4,596,792
U.S. Pat. No. 4,599,230
U.S. Pat. No. 4,599,231
U.S. Pat. No. 4,601,903
U.S. Pat. No. 4,608,251
U.S. Pat. No. 4,676,980
U.S. Pat. No. 4,695,624
U.S. Pat. No. 4,710,111
U.S. Pat. No. 4,741,900
U.S. Pat. No. 4,816,397
U.S. Pat. No. 4,816,567
U.S. Pat. No. 4,826,687
U.S. Pat. No. 5,112,946
U.S. Pat. No. 5,225,539
U.S. Pat. No. 5,336,603
U.S. Pat. No. 5,349,053
U.S. Pat. No. 5,359,046
U.S. Pat. No. 5,413,923
U.S. Pat. No. 5,429,599
U.S. Pat. No. 5,474,981
U.S. Pat. No. 5,484,719
U.S. Pat. No. 5,506,121
U.S. Pat. No. 5,523,088
U.S. Pat. No. 5,530,101
U.S. Pat. No. 5,545,806
U.S. Pat. No. 5,565,203
U.S. Pat. No. 5,565,332
U.S. Pat. No. 5,569,825
U.S. Pat. No. 5,578,453
U.S. Pat. No. 5,585,089
U.S. Pat. No. 5,589,466
U.S. Pat. No. 5,593,972
U.S. Pat. No. 5,605,793
U.S. Pat. No. 5,607,852
U.S. Pat. No. 5,612,487
U.S. Pat. No. 5,614,610
U.S. Pat. No. 5,622,929
U.S. Pat. No. 5,623,057
U.S. Pat. No. 5,625,126
U.S. Pat. No. 5,633,425
U.S. Pat. No. 5,648,081
U.S. Pat. No. 5,661,016
U.S. Pat. No. 5,667,782
U.S. Pat. No. 5,670,488
U.S. Pat. No. 5,703,057
U.S. Pat. No. 5,707,644
U.S. Pat. No. 5,725,863
U.S. Pat. No. 5,747,526
U.S. Pat. No. 5,766,588
U.S. Pat. No. 5,766,588
U.S. Pat. No. 5,770,414
U.S. Pat. No. 5,776,451
U.S. Pat. No. 5,783,196
U.S. Pat. No. 5,807,715
U.S. Pat. No. 5,811,238
U.S. Pat. No. 5,814,295
U.S. Pat. No. 5,814,318
U.S. Pat. No. 5,821,333
U.S. Pat. No. 5,824,544
U.S. Pat. No. 5,830,721
U.S. Pat. No. 5,830,725
U.S. Pat. No. 5,834,252
U.S. Pat. No. 5,837,458
U.S. Pat. No. 5,846,225
U.S. Pat. No. 5,846,233
U.S. Pat. No. 5,851,826
U.S. Pat. No. 5,858,744
U.S. Pat. No. 5,869,451
U.S. Pat. No. 5,871,982
U.S. Pat. No. 5,879,934
U.S. Pat. No. 5,885,793
U.S. Pat. No. 5,888,502
U.S. Pat. No. 5,888,773
U.S. Pat. No. 5,889,157
U.S. Pat. No. 5,891,432
U.S. Pat. No. 5,910,306
U.S. Pat. No. 5,914,123
U.S. Pat. No. 5,916,771
U.S. Pat. No. 5,922,326
U.S. Pat. No. 5,932,210
U.S. Pat. No. 5,933,819
U.S. Pat. No. 5,939,598
U.S. Pat. No. 5,942,242
U.S. Pat. No. 5,955,331
U.S. Pat. No. 5,976,544
U.S. Pat. No. 5,976,546
U.S. Pat. No. 5,980,898
U.S. Pat. No. 5,985,318
U.S. Pat. No. 6,004,807
U.S. Pat. No. 6,005,079
U.S. Pat. No. 6,034,298
U.S. Pat. No. 6,103,493
U.S. Pat. No. 6,129,920
U.S. Pat. No. 6,136,313
U.S. Pat. No. 6,136,320
U.S. Pat. No. 6,180,110
U.S. Pat. No. 6,194,551
U.S. Pat. No. 6,194,560
U.S. Pat. No. 6,200,959
U.S. Pat. No. 6,207,157
U.S. Pat. No. 6,210,663
U.S. Pat. No. 6,210,963
U.S. Pat. No. 6,228,640
U.S. Pat. No. 6,248,334
U.S. Pat. No. 6,248,565
U.S. Pat. No. 6,254,873
U.S. Pat. No. 6,258,358
U.S. Pat. No. 6,270,765
U.S. Pat. No. 6,277,375 B1
U.S. Pat. No. 6,287,571
U.S. Pat. No. 6,299,881
U.S. Pat. No. 6,303,130
U.S. Pat. No. 6,303,755
U.S. Pat. No. 6,309,646
U.S. Pat. No. 6,328,958
U.S. Pat. No. 6,339,068
U.S. Pat. No. 6,339,086
U.S. Pat. No. 6,348,449
U.S. Pat. No. 6,348,450
U.S. Pat. No. 6,359,054
U.S. Pat. No. 6,365,161
U.S. Pat. No. 6,383,795
U.S. Pat. No. 6,395,964
U.S. Pat. No. 6,406,705
U.S. Pat. No. 6,410,690
U.S. Pat. No. 6,413,768
U.S. Pat. No. 6,444,805
U.S. Patent Pub. 20020055618
EP 0598877
EP 239,400
EP 307,434
EP 367,166
EP 439,095
EP 519,596
EP 592,106
WO 00/75308
WO 00/75308
WO 91/06570
WO 91/09967
WO 91/10741
WO 92/01047
WO 93/21232
WO 94/04678
WO 94/25591
WO 96/04388
WO 96/33735
WO 96/33735
WO 96/34096
WO 96/34096
WO 97/33899
WO 97/34911
WO 98/16654
WO 98/24893
WO 98/24893
WO 98/46645
WO 99/16873
WO 99/16873
WO 99/23105
WO 99/50433
WO 99/54452
Alberti et al., Vaccine, 16(6), 608-612, 1998.
Antibodies: A Laboratory Manual, Cold Spring Harbor Laboratory, Cold Spring Harbor Press, Cold Spring Harbor, N.Y., 1988.
Arnon et al., In: Monoclonal antibodies and cancer therapy, Reisfeld et al. (eds.), 243-256, Alan R. Liss, Inc., 1985.
Ashkenazi et al., PNAS, 88:10535-10539, 1991.
Balicki and Beutler, Medicine, 81(1):69-86. 2002.
Bangham et al., J. Mol. Biol., 13:238, 1965.
Barany and Merrifield, In: The peptides, Gross and Meienhofer (eds.), Academic Press, NY, 1-284, 1979.
Barletta et al., Res. Microbiol, 141:931-940, 1990.
Barry et al., Nature, 377:632-635, 1995.
Bergstrom et al., Scand. J. Infect. Dis., 77:102-107, 1991.
Bilbao et al.,FASEB J, 11(8):624-634, 1997.
Brayton et al., Ann. NY, Acad. Sci., 849, 369-71, 1998.
Caplen et al., Gene Ther., 6(3):454-459, 1999.
Casjens et al., Mol. Microbiol., 35(3):490-516, 2000.
Celluzzi and Falo, J. Invest. Dermatol., 108(5):716-720, 1997.
Coffey et al., Science, 282(5392):1332-1334, 1998.
Coney et al., Vaccine, 12(16):1545-1550, 1994.
Croke et al., Infection 1 mm., 68(2), 658-663, 2000.
Curtiss et al., Curr. Top. Microbiol. Immunol., 146:35-49, 1989.
Davies et al., Biotech. and Bioengin., 74(4): 288-294, 2001.
Deamer and Nichols, Proc. Natl. Acad. Sci. USA, 80(1):165-168, 1983.
Dean et al., Genome Res., 11(7):1156-1166, 2001.
Denardo et al., Clin. Cancer Res., 4:2483-2490, 1998.
Dougan et al., Para. Immunol., 9:151-160, 1987.
Farlie et al., Acta Obstet. Gynecol. Scand., 77(1):131-133, 1998.
Fell et al., J. Immunol., 146:2446-2452, 1991.
Feng et al., Nat. Biotechnol., 15(9):866-70, 1997.
Fikrig et al., Science, 250(4980):553-556, 1990.
Fisher et al., Hum. Gene Ther., 7(17):2079-2087, 1996.
Fraser et al., Nature, 390(6660):580-586, 1997.
Gayle and Ringdahl, Amer. Fam. Physic., 64(3):461-466, 2001.
Gentz et al., Proc. Natl. Acad. Sci. USA, 86:821-824, 1989.
Gerber, Pediatr. Infect. Dis. J, 18(9):825-826, 1999.
Gillies et al. J, Immunol. Methods, 125:191-202, 1989.
Gillies et al., PNAS, 89:1428-1432, 1992.
Glenn et al., Exp. Opin. Invest. Drugs, 8:797-805, 1999.
Gnant et al., Cancer Res., 59(14):3396-403, 1999.
Goodman and Gilman's The Pharmacological Basis of Therapeutics, 6th Ed., Goodman et al., (Eds.), 1080-1248; Macmillan Publishing Co., NY, 1980.
Gregoriadis, Drug Carriers In Biology And Medicine, G. Gregoriadis (ed.), 287-341, 1979.
Hagman et al., Infection 1 mm., 66(6):2674-2683, 1998.
Hale, Res. Microbiol., 141(7-8):913-919, 1990.
Hansson et al., J. Mol. Biol., 287:265, 1999.
Harayama, Trends Biotechnol., 16:76, 1998.
Hellstrom et al., In: Controlled drug delivery, 2d Ed., Robinson et al. (eds.), 623-653, Marcel Dekker, Inc., 1987.
Hercogova, Int. J. Dermatol., 40(9):547-550, 2001.
Hinnebusch and Barbour, J. Bacteriol., 174:5251-5257, 1992.
Holzer et al. Virology, 253(1):107-114, 1999.
Hosaka et al., J. Virol., 46(3):1014-1017, 1983.
Huang et al., Virology, 97:212-217, 1979.
Imai et al., Nephrologie, 19(7):397-402, 1998.
Iyer et al., Infect. Immun., 68(3):1714-1718, 2000.
Jensen et al., Infection 1 mm., 66(4):1507-1512, 1998.
Jespers et al., Bio/technology, 12:899-903, 1988.
Kaneda et al., Science, 243:375-378, 1989.
Klein et al., Nature, 327:70-73, 1987.
Knappik et al., Biotechniques, 17(4):754-761, 1994.
Kobayishi et al., Immunochemistry, 10:73, 1973.
Komacki and Oliver, Infect. Immun., 66(9):4115-4122, 1998.
Kyte and Doolittle, J Mol Biol, 157(1):105-32, 1982.
Levine et al.,Microecol. Ther., 19:23-32, 1990.
Liu and Huang, J Control Release, 78(1-3):259-66, 2002.
Lonberg and Huszar, Int. Rev. Immunol., 13:65-93, 1995.
Lorenzo and Blasco, BioTechniques, 24:308, 1998.
Lowrie et al., Vaccine, 12(16):1537-1540, 1994.
Luft et al., J. Infect. Dis., 185(Suppl 1):S46-51, 2001.
Lundstrom, J. Recept Signal Transduct. Res., 19(1-4):673-686, 1999.
Manoutcharian et al., Immunol. Lett., 62(3):131-136, 1998.
Manthorpe et al., Hum. Gene. Ther., 4(4):419-431, 1993.
Merrifield, Science, 232:341-347, 1986.
Moore, Trends Pharmacol. Sci., 15(4):124-129, 1994.
Morris et al., Gastroenterol, 103:699-702, 1992.
Morrison, Science, 229:1202, 1985.
Mullis et al., Biotechnology, 24:17-27, 1992.
Mullis, Sci. Am., 262(4):56-61, 64-65, 1990.
Munson and Pollard, Anal. Biochem., 107:220, 1980.
Muyldermans et al., Trends Biochem. Sci., 26:230, 2001.
Naramura et al., Immunol. Lett., 39:91-99, 1994.
Neumann et al., Proc. Natl. Acad. Sci. USA, 96(16):9345-9350, 1999.
Nicolau et al., Methods Enzymol., 149:157-176, 1987.
Nuttall et al., Cur. Pharm. Biotech., 1:253, 2000.
Padlan, Molec. Immunol., 28(4/5):489-498, 1991.
Patten et al., Curr. Opinion Biotechnol., 8:724-33, 1997.
Peterson et al., Bioconjug. Chem., 10:553, 1999.
Piedrafita et al., J. Immunol., 163(3): 1467-1472, 1999.
Poland and Jacobson, Vaccine, 19(17-19):2303-2308, 2001.
Rahn, Infect. Dis. Clin. NA., 15(1):171-187, 2001.
Ravid et al., In: DNA transfer to cultured cells, Wiley and Sons, 1998
Reichmann and Muyldermans, J. Immunol. Meth., 231:25, 1999.
Remington's Pharmaceutical Sciences, 15^{th} ed., pages 1035-1038 and 1570-1580, Mack Publishing Company, Easton, Pa., 1980.
Riechmann et al., Nature, 332:323-327, 1988.
Robbins et al., Trends Biotech., 16(1):35-40, 1998.
Roguska et al., Proc. Natl. Acad. Sci. USA, 91:969-973, 1994.
Rose et al., J. Rheumat., 28(11):2555-2557, 2001.
Rouse et al., J. Virol., 68(9):5685-5689, 1994.
Sambrook et al., Molecular Cloning: A Laboratory Manual, Cold Spring Harbor Laboratory; 3rd edition, 2001.
Sanford et al., Technique, 3:3-16, 1991.
Shields et al., J. Biol. Chem., 277(30):26733-26740, 2001.
Slack, In: Oxford Textbook of Medicine, 2d Ed., Weatherall et al., (Eds.) 1:5.35, Oxford University Press, Oxford/Melbourne/New York, 1987.
Smooker et al., Vaccine, 18(23):2533-2540, 2000.
Steinman et al., Hum Immunol, 60(7):562-567, 1999
Stemmer et al., Gene, 154(1):49-53, 1995.
Stewart and Young, In: Solid phase peptide synthesis, 2d. ed., Pierce Chemical Co. 1984.
Straubinger et al., Vaccine, 20:181-193, 2002.
Studnicka et al., Protein Eng., 7(6):805-814,1994.
Sykes and Johnston, DNA Cell. Biol., 18:521-531, 1999.
Sykes et al., Vaccine, 20(17-18):2382-2395, 2002.
Szoka and Papahadjopoulos, Proc. Natl. Acad. Sci. USA, 75:4194-4198, 1978.
Takahashi et al., J. Immunol., 6:1567-1574, 1994
Tam et al., J. Am. Chem. Soc., 105:6442, 1983.
Tang et al., Nature, 356:152-154, 1992.
Thanassi and Schoen, Ann. Intern Med., 132(8):661-668, 2000.
Thorpe et al., Immunol. Rev., 65:119-138, 1982.
Thorpe, In: Monoclonal antibodies '84: Biological and clinical applications, Pinchera et al. (eds.), 475-506, 1985.
Trollmo et al., J. Immunol., 166(8):5286-5291, 2001.
Van Damme et al., Gastroenterology, 103(2):520-531, 1992.
Van Solingen and Evans, Curr. Opin. Rheumatol., 13(4):293-299, 2001.
Vaz et al., Infection and 1 mm., 69(12):7437-7444, 2001.
Vil et al., PNAS, 89:11337-11341, 1992.
Viret et al., Mol. Microbiol., 7(2):239-252, 1993.
Watt et al., Proc. Natl. Acad. Sci., 83(2):3166-3170, 1986.
Wilson et al., Cell, 37:767, 1984.
Wormser, Infection, 24(2):203-207, 1996.
Xiang and Ertl, Immunity, 2(2):129-135, 1995.
Xiang et al., J Virol, 199(1):132-140, 1994.
Zhang et al, Cell, 89(2):275-285, 1997.
Zheng et al., J. Immunol., 154:5590-5600, 1995.
Zimmerman et al.,Nucl. Med. Biol., 26:943-50, 1999.

1-30. (Canceled)

31. A vaccine composition comprising at least one Borrelia antigen or fragment thereof or at least one polynucleotide encoding a Borrelia antigen or a fragment thereof.

32. The vaccine composition of claim 31, further defined as a genetic vaccine, a polypeptide vaccine, a cell-mediated vaccine, an attenuated pathogen vaccine, a live vector vaccine, an edible vaccine, a killed pathogen vaccine, a purified sub-unit vaccine, a conjugate vaccine, a virus-like particle vaccine, or a humanized antibody vaccines.

33. The vaccine composition of claim 32, further defined as comprising a polynucleotide encoding at least one Borrelia antigen or fragment thereof.

34. The vaccine composition of claim 32, further defined as comprising at least one Borrelia antigen or a fragment thereof.

35. The vaccine composition of claim 31, further defined as comprising at least one polynucleotide encoding a Borrelia antigen or fragment thereof.

36. The vaccine composition of claim 35, further defined as comprising at least two polynucleotides encoding different Borrelia antigens or fragments thereof.

37. The vaccine composition of claim 36, further defined as comprising at least three polynucleotides encoding different Borrelia antigens or fragments thereof.

38. The vaccine composition of claim 37, further defined as comprising at least four polynucleotides encoding different Borrelia antigens or fragments thereof.

39. The vaccine composition of claim 35, wherein the polynucleotide encoding the Borrelia antigen or fragment thereof encodes a polypeptide comprising the amino acid sequence of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, SEQ ID NO:112, SEQ ID NO:114, SEQ ID NO:117, SEQ ID NO:119, SEQ ID NO:121, SEQ ID NO:123, SEQ ID NO:125, SEQ ID NO:127, SEQ ID NO:129, SEQ ID NO:131, SEQ ID NO:133, SEQ ID NO:135, SEQ ID NO:137, or SEQ ID NO:139, or a fragment thereof.

40. The vaccine composition of claim 35, wherein the polynucleotide encoding a Borrelia antigen or fragment thereof comprises the polynucleotide sequence of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:101, SEQ ID NO:103, SEQ ID NO:105, SEQ ID NO:107, SEQ ID NO:109, SEQ ID NO:11, SEQ ID NO:113, SEQ ID NO:115, SEQ ID NO:116, SEQ ID NO:118, SEQ ID NO:120, SEQ ID NO:122, SEQ ID NO:124, SEQ ID NO:126, SEQ ID NO:128, SEQ ID NO:130, SEQ ID NO:132, SEQ ID NO:134, SEQ ID NO:136, or SEQ ID NO:138, or a fragment thereof.

41. The vaccine composition of claim 36, wherein the at least two polynucleotides encoding different Borrelia antigens comprises the polynucleotide sequence of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:101, SEQ ID NO:103, SEQ ID NO:105, SEQ ID NO:107, SEQ ID NO:109, SEQ ID NO:111, SEQ ID NO:113, SEQ ID NO:115, SEQ ID NO:116, SEQ ID NO:118, SEQ ID NO:120, SEQ ID NO:122, SEQ ID NO:124, SEQ ID NO:126, SEQ ID NO:128, SEQ ID NO:130, SEQ ID NO:132, SEQ ID NO:134, SEQ ID NO:136, or SEQ ID NO:138, or a fragment thereof.

42. The vaccine composition of claim 37, wherein the at least three polynucleotides encoding different Borrelia antigens comprises the polynucleotide sequence of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:101, SEQ ID NO:103, SEQ ID NO:105, SEQ ID NO:107, SEQ ID NO:109, SEQ ID NO:11, SEQ ID NO:113, SEQ ID NO:115, SEQ ID NO:116, SEQ ID NO:118, SEQ ID NO:120, SEQ ID NO:122, SEQ ID NO:124, SEQ ID NO:126, SEQ ID NO:128, SEQ ID NO:130, SEQ ID NO:132, SEQ ID NO:134, SEQ ID NO:136, or SEQ ID NO:138, or a fragment thereof.

43. The vaccine composition of claim 38, wherein the at least four polynucleotides encoding different Borrelia antigens comprises the polynucleotide sequence of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, SEQ ID NO:87, SEQ ID NO:89, SEQ ID NO:91, SEQ ID NO:93, SEQ ID NO:95, SEQ ID NO:97, SEQ ID NO:99, SEQ ID NO:101, SEQ ID NO:103, SEQ ID NO:105, SEQ ID NO:107, SEQ ID NO:109, SEQ ID NO:111, SEQ ID NO:113, SEQ ID NO:115, SEQ ID NO:116, SEQ ID NO:118, SEQ ID NO:120, SEQ ID NO:122, SEQ ID NO:124, SEQ ID NO:126, SEQ ID NO:128, SEQ ID NO:130, SEQ D NO:132, SEQ ID NO:134, SEQ ID NO:136, or SEQ ID NO:138, or a fragment thereof.

44. The vaccine composition of claim 31, further defined as comprising at least one Borrelia antigen or fragment thereof in a pharmaceutically acceptable carrier.

45. The vaccine composition of claim 44, further defined as comprising at least two different Borrelia antigens or fragments thereof in a pharmaceutically acceptable carrier.

46. The vaccine composition of claim 45, further defined as comprising at least three different Borrelia antigens or fragments thereof in a pharmaceutically acceptable carrier.

47. The vaccine composition of claim 46, further defined as comprising at least four different Borrelia antigens or fragments thereof in a pharmaceutically acceptable carrier.

48. The vaccine composition of claim 44, wherein the Borrelia antigen or fragments thereof has an amino acid sequence of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, SEQ ID NO:112, SEQ ID NO:114, SEQ ID NO:117, SEQ ID NO:119, SEQ ID NO:121, SEQ ID NO:123, SEQ ID NO:125, SEQ ID NO:127, SEQ ID NO:129, SEQ ID NO:131, SEQ ID NO:133, SEQ ID NO:135, SEQ ID NO:137, or SEQ ID NO:139 or fragments thereof.

49. The vaccine composition of claim 45, wherein the at least two different Borrelia antigens or fragments thereof have an amino acid sequence of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, SEQ ID NO:112, SEQ ID NO:114, SEQ ID NO:117, SEQ ID NO:119, SEQ ID NO:121, SEQ ID NO:123, SEQ ID NO:125, SEQ ID NO:127, SEQ ID NO:129, SEQ ID NO:131, SEQ ID NO:133, SEQ ID NO:135, SEQ ID NO:137, or SEQ ID NO:139, or fragments thereof.

50. The vaccine composition of claim 46, wherein the at least three different Borrelia antigens or fragments thereof have an amino acid sequence of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, SEQ ID NO:112, SEQ ID NO:114, SEQ ID NO:117, SEQ ID NO:119, SEQ ID NO:121, SEQ ID NO:123, SEQ ID NO:125, SEQ ID NO:127, SEQ ID NO:129, SEQ ID NO:131, SEQ ID NO:133, SEQ ID NO:135, SEQ ID NO:137, or SEQ ID NO:139, or fragments thereof.

51. The vaccine composition of claim 47, wherein the at least four different Borrelia antigens or fragments thereof have an amino acid sequence of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, SEQ ID NO:112, SEQ ID NO:114, SEQ ID NO:117, SEQ ID NO:119, SEQ ID NO:121, SEQ ID NO:123, SEQ ID NO:125, SEQ ID NO:127, SEQ ID NO:129, SEQ ID NO:131, SEQ ID NO:133, SEQ ID NO:135, SEQ ID NO:137, or SEQ ID NO:139, or fragments thereof.

52-87. (Canceled)
